(19) United States
(12) Patent Application Publication
(10) Pub. No.: US 2011/0190263 A1
(43) Pub. Date: Aug. 4, 2011

(54) COMPOUNDS AND COMPOSITIONS AS MODULATORS OF GPR119 ACTIVITY

(75) Inventors: Mihai Azimioara, La Jolla, CA (US); Christopher Cow, Poway, CA (US); Robert Epple, San Diego, CA (US); Songchun Jiang, San Diego, CA (US); Gerald Lelais, San Diego, CA (US); Baogen Wu, San Diego, CA (US); Daniel Mutnick, San Diego, CA (US)

(73) Assignee: IRM LLC, Hamilton (BM)

(21) Appl. No.: 12/918,803

(22) PCT Filed: Feb. 20, 2009
(86) PCT No.: PCT/US09/34783

§ 371 Date: Feb. 25, 2011

Related U.S. Application Data
(60) Provisional application No. 61/030,805, filed on Feb. 22, 2008.

(51) Int. Cl. A61K 31/519; C12N 5/02; C07D 471/04; A61K 31/506; A61P 3/04; A61P 3/10; A61P 3/06; A61P 9/00; A61P 19/02; A61P 19/10; A61P 9/02; A61P 13/12; A61P 17/00; A61P 1/00

(52) U.S. Cl. 514/210.21; 435/375; 544/279; 514/275; 544/297

(57) ABSTRACT

The invention provides compounds of Formula (I): pharmaceutical compositions comprising such compounds and methods of using such compounds to treat or prevent diseases or disorders associated with the activity of GPR119.

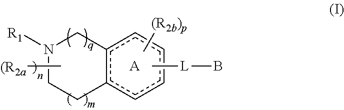

### CROSS-REFERENCE TO RELATED APPLICATIONS

This application claims the benefit of priority to U.S. Provisional Patent Application No. 61/030,805, filed 22 Feb. 2008. The full disclosure of this application is incorporated herein by reference in its entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of GPR119.

2. Background

GPR119 is a G-protein coupled receptor (GPCR) that is mainly expressed in the pancreas, small intestine, colon and adipose tissue. The expression profile of the human GPR119 receptor indicates its potential utility as a target for the treatment of obesity and diabetes. The novel compounds of this invention modulate the activity of GPR119 and are, therefore, expected to be useful in the treatment of GPR119-associated diseases or disorders such as, but not limited to, diabetes, obesity and associated metabolic disorders.

### SUMMARY OF THE INVENTION

In one aspect, the present invention provides a compound of Formula I:

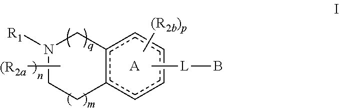

in which:

A is a 6 member saturated, partially unsaturated or aromatic ring system containing at least one heteroatom or moiety selected from N and C(O); [symbol] represents a single or double bond and ring A can be, for example, one of the following structures:

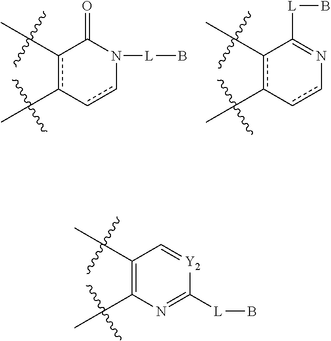

wherein Y₂ is selected from CH and N;

B is selected from C₆₋₁₀aryl, C₁₋₁₀heteroaryl, C₃₋₁₂cycloalkyl and C₃₋₈ heterocycloalkyl; wherein said aryl, heteroaryl, cycloalkyl or heterocycloalkyl is substituted with one to three R₃ radicals;

n is selected from 0, 1, 2 and 3;

p is selected from 0, 1 and 2;

q is selected from 0 and 1;

m is selected from 1 and 2;

L is selected from a bond, C₁₋₆alkylene, —X₁OX₂—, —X₁NR₄X₂—, —OX₃O— and —X₆X₂—; wherein R₄ is selected from hydrogen and C₁₋₄alkyl; X₁ is selected from a bond, C₁₋₄alkylene and C₃₋₈heterocycloalkyl-C₀₋₁alkyl; X₂ is selected from a bond and C₁₋₄alkylene; X₃ is C₁₋₄alkylene; and X₆ is a 5 member heteroaryl;

R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;

R₂ₐ and R_{2b} are independently selected from halo, cyano, hydroxy, C₁₋₄ alkyl, amino, nitro, —C(O)OR₅ₑ, —C(O)R₅, and —NR₅ₑR_{5f}; wherein R₅ₑ and R_{5f} are independently selected from hydrogen, C₁₋₆alkyl, C₃₋₁₂cycloalkyl, halo-substituted-C₁₋₆alkyl, halo-substituted-C₃₋₈cycloalkyl, C₆₋₁₀aryl and C₁₋₁₀heteroaryl; wherein said aryl or heteroaryl of R₅ₑ or R_{5f} can be optionally substituted with 1 to 3 radicals independently selected from C₁₋₆alkyl, C₁₋₆alkoxy, halo-substituted-C₁₋₆alkyl and halo-substituted-C₁₋₆alkoxy;

R₃ is selected from C₁₋₁₀heteroaryl, C₆₋₁₀aryl, C₃₋₈heterocycloalkyl, halo, —C(O)OR₆ₐ, —C(O)R₆ₐ, —S(O)₀₋₂R₆ₐ, —C(O)R₇, —C(O)X₅NR₆ₐC(O)OR_{6b}, —C(S)OR₆ₐ, —C(S)R₆ₐ, —C(S)R₇ and —C(S)X₅NR₆ₐC(O)OR_{6b}; wherein X₅ is selected from a bond and C₁₋₆alkylene; or two adjacent R₃ groups together with the carbon atom to which they are attached form a C₃₋₈heterocycloalkyl optionally substituted with a group selected from —C(O)OR_{6c} and —R_{6d}; R₆ₐ, R_{6b} and R_{6c} are independently selected from hydrogen, C₁₋₆alkyl, halo-substituted-C₁₋₆alkyl, C₃₋₁₂cycloalkyl optionally substituted with C₁₋₄alkyl, halo-substituted-C₁₋₆cycloalkyl; R_{6d} is C₁₋₁₀heteroaryl optionally substituted with C₁₋₄alkyl; R₇ is selected from C₁₋₈alkyl, C₃₋₈cycloalkyl, C₆₋₁₀aryl, C₁₋₁₀heteroaryl, halo-substituted C₁₋₈alkyl, halo-substituted-C₃₋₈cycloalkyl, halo-substituted-C₆₋₁₀aryl and halo-substituted-C₆₋₁₀heteroaryl; wherein said aryl, heteroaryl or heterocycloalkyl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐC(O)R₈ₐ, —X₅ₐR₉, C₁₋₆alkyl, C₁₋₆alkoxy, halo-substituted-C₁₋₆alkyl and halo-substituted-C₁₋₆alkoxy; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of GPR119 activity can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which GPR119 activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be straight-chained, branched, cyclic or spiro. C₁₋₆alkoxy includes methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group.

“Heteroaryl” is as defined for aryl where one or more of the ring members are a heteroatom. For example, C₁₋₁₀heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, 1H-pyridin-2-onyl, 6-oxo-1,6-dihydro-pyridin-3-yl, etc. Also, a 5 member heteroaryl is used, for example to define X6. A 5 member heteroaryl includes imidazole (see examples G17 and G18).

“two adjacent R₃ groups together with the carbon atom to which they are attached form a C₃₋₈heterocycloalkyl” means, for example, the formation of 1,2,3,4-tetrahydroisoquinoline such as found in example I1.

“C₆₋₁₀arylC₀₋₄alkyl” means an aryl as described above connected via a alkylene grouping. For example, C₆₋₁₀arylC₀₋₄alkyl includes phenethyl, benzyl, etc. Heteroaryl also includes the N-oxide derivatives, for example, pyridine N-oxide derivatives with the following structure:

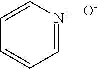

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, 2-oxo-pyrrolidin-1-yl, 2-oxo-piperidin-1-yl, etc.

“C₃₋₈heterocycloalkyl-C₀₋₁alkyl” as defined for X₁, can be for example, the following moiety (such as is found in examples G2-G13 of table 4):

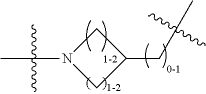

GPR119 means G protein-coupled receptor 119 (GenBank® Accession No. AAP72125) is also referred to in the literature as RUP3 and GPR116. The term GPR119 as used herein includes the human sequences found in GeneBank accession number AY288416, naturally-occurring allelic variants, mammalian orthologs, and recombinant mutants thereof.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of GPR119 activity can prevent, inhibit or ameliorate the pathology and/or symptomology of the diseases, which method comprises administering to the animal a therapeutically effective amount of a compound of Formula I.

In one embodiment, with reference to compounds of Formula I, are compounds Formula Ia, Ib, Ic, Id and Ie:

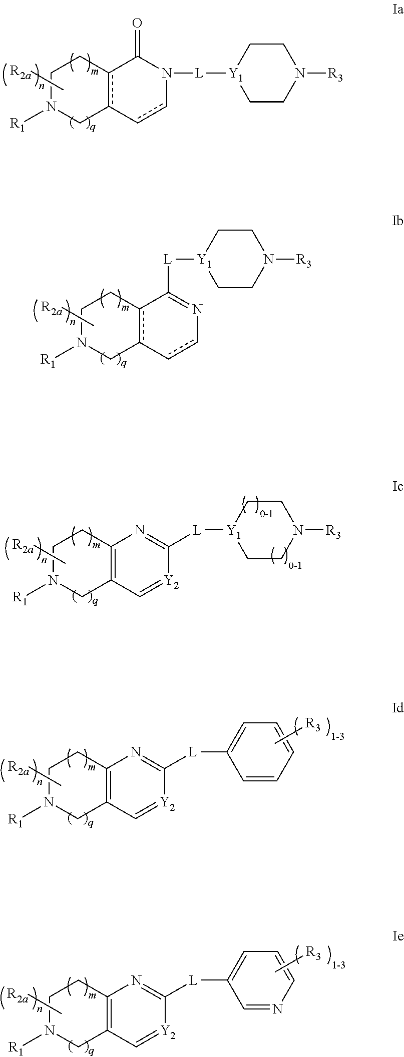

in which:

n is selected from 0, 1, 2 and 3;

q is selected from 0 and 1;

m is selected from 1 and 2;

L is selected from a bond, C₁₋₆alkylene, —X₁OX₂—, —X₁NR₄X₂—, —OX₃O— and —X₆X₂—; wherein R₄ is selected from hydrogen and C₁₋₄alkyl; X₁ is selected from a bond, C₁₋₄alkylene and C₃₋₈heterocycloalkyl-C₀₋₁alkyl; X₂ is selected from a bond and C₁₋₄alkylene; X₃ is C₁₋₄alkylene; and X₆ is a 5 member heteroaryl;

R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀ aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;

R₂ₐ is selected from halo, cyano, hydroxy, C₁₋₄alkyl, amino, nitro, —C(O)OR₅ₑ, —C(O)R₅, and —NR₅ₑR_{5f}; wherein R₅ₑ and R_{5f} are independently selected from hydrogen, C₁₋₆alkyl, C₃₋₁₂cycloalkyl, halo-substituted-C₁₋₆alkyl, halo-substituted-C₃₋₈ cycloalkyl, C₆₋₁₀aryl and C₁₋₁₀heteroaryl; wherein said aryl or heteroaryl of R₅ₑ or R_{5f} can be optionally substituted with 1 to 3 radicals independently selected from C₁₋₆alkyl, C₁₋₆ alkoxy, halo-substituted-C₁₋₆alkyl and halo-substituted-C₁₋₆alkoxy;

R₃ is selected from C₁₋₁₀heteroaryl, C₆₋₁₀aryl, C₃₋₈heterocycloalkyl, halo, —C(O)OR₆ₐ, —C(O)R₆ₐ, —S(O)₀₋₂R₆ₐ, —C(O)R₇, —C(O)X₅NR₆ₐC(O)OR_{6b}, —C(S)OR₆ₐ, —C(S)R₆ₐ, —C(S)R₇ and —C(S)X₅NR₆ₐC(O)OR_{6b}; wherein X₅ is selected from a bond and C₁₋₆alkylene; or two adjacent R₃ groups together with the carbon atom to which they are attached form a C₃₋₈heterocycloalkyl optionally substituted with a group selected from —C(O)OR_{6c} and —R_{6d}; R₆ₐ, R_{6b} and R_{6c} are independently selected from hydrogen, C₁₋₆alkyl, halo-substituted-C₁₋₆alkyl, C₃₋₁₂cycloalkyl optionally substituted with C₁₋₄alkyl, halo-substituted-C₁₋₆cycloalkyl; R_{6d} is C₁₋₁₀heteroaryl optionally substituted with C₁₋₄alkyl; R₇ is selected from C₁₋₈alkyl, C₃₋₈cycloalkyl, C₆₋₁₀aryl, C₁₋₁₀heteroaryl, halo-substituted C₁₋₈alkyl, halo-substituted-C₃₋₈cycloalkyl, halo-substituted-C₆₋₁₀aryl and halo-substituted-C₆₋₁₀heteroaryl; wherein said aryl, heteroaryl or heterocycloalkyl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐC(O)R₈ₐ, —X₅ₐR₉, C₁₋₆alkyl, C₁₋₆alkoxy, halo-substituted-C₁₋₆alkyl and halo-substituted-C₁₋₆alkoxy; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; and

Y₁ and Y₂ are independently selected from CH and N; wherein the dotted lines of formulae Ia or Ib independently indicate the presence of a double or single bond.

In another embodiment, L is selected from a bond, —(CH₂)₁₋₄—, —O(CH₂)₀₋₄—, —CH₂NH(CH₂)₀₋₂—, —NH(CH₂)₁₋₃—, —N(CH₃)(CH₂)₁₋₃—, —CH₂O(CH₂)₁₋₂—, —O(CH₂)₂O— and —X₆(CH₂)₀₋₁; wherein X₆ is imidazole; or a moiety of formula II:

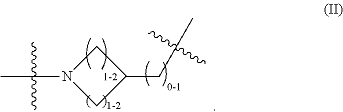

In another embodiment, R₁ is selected from methyl-sulfonyl, butyl-sulfonyl, phenyl-sulfonyl, isopropyl-sulfonyl, ethyl-sulfonyl, ethenyl-sulfonyl, isopropoxy-carbonyl, benzoxy-carbonyl, ethoxy-carbonyl, methoxy-carbonyl, t-butoxy-carbonyl and trifluoromethyl-sulfonyl.

In another embodiment, R₃ is selected from halo, t-butoxy-carbonyl, t-butoxy-carbonyl-amino-methyl, isopropoxy-carbonyl, 3-isopropyl-(1,2,4-oxadiazol-5-yl), (1-methylcyclopropoxy)carbonyl, azetidin-1-yl, pyridinyl, piperidinyl, pyrimidinyl, pyrazolyl, benzoxycarbonyl and cyclopropoxy-carbonyl; wherein said azetidin-1-yl, pyridinyl, piperidinyl, cyclopropoxy or pyrimidinyl can be optionally substituted by 1 to 2 radicals independently selected from methyl, isopropyl, ethyl and pyrimidinyl optionally substituted with ethyl; or two adjacent R₃ groups together with the carbon atom to which they are both attached form 1-(tert-butoxycarbonyl)piperidin-4-yl.

In another embodiment, are compounds selected from: Isopropyl 4-(3-(1,2,3,4-tetrahydro-2-methanesulfonyl-5-oxo-2,6-naphthyridin-6(5H)-yl)propyl)piperidine-1-carboxylate; isopropyl 4-(3-(1,2,3,4-tetrahydro-2-methanesulfonyl-2,6-naphthyridin-5-yloxy)propyl)piperidine-1-carboxylate; isopropyl 4-(3-(1,2,3,4,4a,7,8,8a-octahydro-2-methanesulfonyl-2,6-naphthyridin-5-yloxy)propyl)piperidine-1-carboxylate; isopropyl 4-(6-(methylsulfonyl)-5,6,7,8-tetrahydro-2,6-naphthyridin-1-yloxy)piperidine-1-carboxylate; isopropyl 4-(6-(methylsulfonyl)-1-oxooctahydro-2,6-naphthyridin-2(1H)-yl)piperidine-1-carboxylate; isopropyl 4-((6-(methylsulfonyl)-1-oxo-5,6,7,8-tetrahydro-2,6-naphthyridin-2(1H)-yl)methyl)piperidine-1-carboxylate; isopropyl 4-(4-(6-(methylsulfonyl)-1-oxo-5,6,7,8-tetrahydro-2,6-naphthyridin-2(1H)-yl)butyl)piperidine-1-carboxylate; isopropyl 4-(4-(6-(methylsulfonyl)-3,4,4a,5,6,7,8,8a-octahydro-2,6-naphthyridin-1-yloxy)butyl)piperidine-1-carboxylate; isopropyl 4-(4-(6-(methylsulfonyl)-5,6,7,8-tetrahydro-2,6-naphthyridin-1-yloxy)butyl)piperidine-1-carboxylate; tert-Butyl 4-(((6-(methylsulfonyl)-5,6,7,8-tetrahydropyrido[4,3-d]pyrimidin-2-yl)methylamino)methyl)piperidine-1-carboxylate; tert-butyl 4-(2-((6-(methylsulfonyl)-5,6,7,8-tetrahydropyrido[4,3-d]pyrimidin-2-yl)methylamino)ethyl)piperidine-1-carboxylate; 2-(3-bromophenyl)-N-((6-(methylsulfonyl)-5,6,7,8-tetrahydropyrido[4,3-d]pyrimidin-2-yl)methyl)ethanamine; tert-butyl 4-((6-(methylsulfonyl)-5,6,7,8-tetrahydropyrido[4,3-d]pyrimidin-2-yl)methylamino)benzylcarbamate; 1-Methylcyclopropyl 4-(2-((6-(methylsulfonyl)-5,6,7,8-tetrahydropyrido[4,3-d]pyrimidin-2-yl)methoxy)ethyl)piperidine-1-carboxylate; 3-Isopropyl-5-(4-(3-(6-(methylsulfonyl)-5,6,7,8-tetrahydropyrido[4,3-d]pyrimidin-2-yloxy)propyl)piperidin-1-yl)-1,2,4-oxadiazole; 1-Methylcyclopropyl 4-(3-(6-(methylsulfonyl)-5,6,7,8-tetrahydropyrido[4,3-d]pyrimidin-2-yloxy)propyl)piperidine-1-carboxylate; 2-(3-(1-(5-Ethylpyrimidin-2-yl)piperidin-4-yl)propoxy)-6-(methylsulfonyl)-5,6,7,8-tetrahydropyrido[4,3-d]pyrimidine; N-(3-(1-(3-isopropyl-1,2,4-oxadiazol-5-yl)piperidin-4-yl)propyl)-6-(methylsulfonyl)-5,6,7,8-tetrahydropyrido[4,3-d]pyrimidin-2-amine; N-(3-(1-(5-Ethylpyrimidin-2-yl)piperidin-4-yl)propyl)-6-(methylsulfonyl)-5,6,7,8-tetrahydropyrido[4,3-d]pyrimidin-2-amine; N-(3-(1-(5-Ethylpyrimidin-2-yl)piperidin-4-yl)propyl)-N-methyl-6-(methylsulfonyl)-5,6,7,8-tetrahydropyrido[4,3-d]pyrimidin-2-amine; 1-methylcyclopropyl 4-(3-(6-(methylsulfonyl)-5,6,7,8-tetrahydropyrido[4,3-d]pyrimidin-2-ylamino)propyl)piperidine-1-carboxylate; 1-methylcyclopropyl 4-(3-(methyl(6-(methylsulfonyl)-5,6,7,8-tetrahydropyrido[4,3-d]pyrimidin-2-yl)amino)propyl)piperidine-1-carboxylate; 2-(2-(1-(5-ethylpyrimidin-2-yl)piperidin-4-yloxy)ethoxy)-6-(methylsulfonyl)-5,6,7,8-tetrahydropyrido[4,3-d]pyrimidine; 2-(3-(1-(5-Ethylpyrimidin-2-yl)piperidin-4-yl)propoxy)-6-(methylsulfonyl)-5,6,7,8-tetrahydro-1,6-naphthyridine; 5-ethyl-2-(4-{[(2S)-1-{6-methane sulfonyl-5H,6H,7H,8H-pyrido[4,3-d]pyrimidin-2-yl}pyrrolidin-2-yl]methoxy}piperidin-1-yl)pyrimidine; benzyl 4-[(1-{6-methanesulfonyl-5H,6H,7H,8H-pyrido[4,3-d]pyrimidin-2-yl}-1H-imidazol-4-yl)methyl]piperidine-1-carboxylate; 1-methylcyclopropyl 3-[(1-{6-methanesulfonyl-5H,6H,7H,8H-pyrido[4,3-d]pyrimidin-2-yl}piperidin-4-yl)methoxy]azetidine-1-carboxylate; 5-[3-({6-methanesulfonyl-5H,6H,7H,8H-pyrido[4,3-d]pyrimidin-2-yl}oxy)propyl]-2-(1H-pyrazol-1-yl)pyridine; 1-methylcyclopropyl 4-[(1-{6-methanesulfonyl-5H,6H,7H,8H-pyrido[4,3-d]pyrimidin-2-yl}-1H-imidazol-4-yl)methyl]piperidine-1-carboxylate; 5-ethyl-2-{3-{(1-{6-methanesulfonyl-5H,6H,7H,8H-pyrido[4,3-d]pyrimidin-2-yl}piperidin-4-yl)methoxy]azetidin-1-yl}pyrimidine; 5-(4-{[(1-{6-methanesulfonyl-5H,6H,7H,8H-pyrido[4,3-d]pyrimidin-2-yl}azetidin-3-yl)oxy]methyl}piperidin-1-yl)-3-(propan-2-yl)-1,2,4-oxadiazole; 3-(4-{[(1-{6-methanesulfonyl-5H,6H,7H,8H-pyrido[4,3-d]pyrimidin-2-yl}azetidin-3-yl)oxy]methyl}piperidin-1-yl)-5-(propan-2-yl)-1,2,4-oxadiazole; 1-methylcyclopropyl(3R,4S)-4-{[(1-{6-methanesulfonyl-5H,6H,7H,8H-pyrido[4,3-d]pyrimidin-2-yl}azetidin-3-yl)oxy]methyl}-3-methoxypiperidine-1-carboxylate; 1-methylcyclopropyl(3R,4R)-4-{[(1-{6-methanesulfonyl-5H,6H,7H,8H-pyrido[4,3-d]pyrimidin-2-yl}azetidin-3-yl)oxy]methyl}-3-methylpiperidine-1-carboxylate; benzyl(2R,4R)-4-{[(1-{6-methanesulfonyl-5H,6H,7H,8H-pyrido[4,3-d]pyrimidin-2-yl}azetidin-3-yl)oxy]methyl}-2-methylpiperidine-1-carboxylate; benzyl 4-{[(1-{6-methanesulfonyl-5H,6H,7H,8H-pyrido[4,3-d]pyrimidin-2-yl}azetidin-3-yl)oxy]methyl}piperidine-1-carboxylate; 2-(5-ethylpyrimidin-2-yl)-5-[(1-{6-methanesulfonyl-5H,6H,7H,8H-pyrido[4,3-d]pyrimidin-2-yl}azetidin-3-yl)oxy]-1,2,3,4-tetrahydroisoquinoline; 5-ethyl-2-(4-{1-[(1-{6-methanesulfonyl-5H,6H,7H,8H-pyrido[4,3-d]pyrimidin-2-yl}azetidin-3-yl)oxy]ethyl}piperidin-1-yl)pyrimidine; 3-(2-{3-[1-(5-ethylpyrimidin-2-yl)piperidin-4-yl]propoxy}-5H,6H,7H,8H-pyrido[4,3-d]pyrimidine-6-sulfonyl)propan-1-ol; tert-butyl 4-(2-{[(3S)-1-{6-methanesulfonyl-5H,6H,7H,8H-pyrido[4,3-d]pyrimidin-2-yl}pyrrolidin-3-yl]oxy}ethyl)piperidine-1-carboxylate; benzyl 2-{3-[1-(5-ethylpyrimidin-2-yl)piperidin-4-yl]propoxy}-5H,6H,7H,8H-pyrido[4,3-d]pyrimidine-6-carboxylate; and 5-ethyl-2-{4-[3-({6-methanesulfonyl-5H,6H,7H,8H-pyrido[4,3-d]pyrimidin-2-yl}oxy)propyl]phenyl}pyrimidine.

Further compounds of the invention are detailed in the Examples and Tables, infra.

The present invention also includes all suitable isotopic variations of the compounds of the invention, or pharmaceutically acceptable salts thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl and ¹²³I. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds of the invention or pharmaceutically acceptable salts thereof can generally be prepared by conventional procedures using appropriate isotopic variations of suitable reagents.

### Pharmacology and Utility

Compounds of the invention modulate the activity of GPR119 and, as such, are useful for treating diseases or disorders in which the activity of GPR119 contributes to the pathology and/or symptomology of the disease. This invention further provides compounds of this invention for use in the preparation of medicaments for the treatment of diseases or disorders in which GPR119 activity contributes to the pathology and/or symptomology of the disease.

The resultant pathologies of Type II diabetes are impaired insulin signaling at its target tissues and failure of the insulin-producing cells of the pancreas to secrete an appropriate degree of insulin in response to a hyperglycemic signal. Current therapies to treat the latter include inhibitors of the β-cell ATP-sensitive potassium channel to trigger the release of endogenous insulin stores, or administration of exogenous insulin. Neither of these achieves accurate normalization of blood glucose levels and both carry the risk of inducing hypoglycemia. For these reasons, there has been intense interest in the development of pharmaceuticals that function in a glucose-dependent action, i.e. potentiators of glucose signaling. Physiological signaling systems which function in this manner are well-characterized and include the gut peptides GLP-1, GIP and PACAP. These hormones act via their cognate G-protein coupled receptor to stimulate the production of cAMP in pancreatic (3-cells. The increased cAMP does not appear to result in stimulation of insulin release during the fasting or pre-prandial state. However, a series of biochemical targets of cAMP signaling, including the ATP-sensitive potassium channel, voltage-sensitive potassium channels and the exocytotic machinery, are modified in such a way that the insulin secretory response to a postprandial glucose stimulus is markedly enhanced. Accordingly, agonists of novel, similarly functioning, β-cell GPCRs, including GPR119, would also stimulate the release of endogenous insulin and consequently promote normoglycemia in Type II diabetes. It is also established that increased cAMP, for example as a result of GLP-1 stimulation, promotes β-cell proliferation, inhibits β-cell death and thus improves islet mass. This positive effect on β-cell mass is expected to be beneficial in both Type II diabetes, where insufficient insulin is produced, and Type I diabetes, where β-cells are destroyed by an inappropriate autoimmune response.

Some β-cell GPCRs, including GPR119, are also present in the hypothalamus where they modulate hunger, satiety, decrease food intake, controlling or decreasing weight and energy expenditure. Hence, given their function within the hypothalamic circuitry, agonists or inverse agonists of these receptors mitigate hunger, promote satiety and therefore modulate weight.

It is also well-established that metabolic diseases exert a negative influence on other physiological systems. Thus, there is often the codevelopment of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which clearly occur secondary to diabetes (e.g. kidney disease, peripheral neuropathy). Thus, it is expected that effective treatment of the diabetic condition will in turn be of benefit to such interconnected disease states.

In an embodiment of the invention is a method for treatment of a metabolic disease and/or a metabolic-related disorder in an individual comprising administering to the individual in need of such treatment a therapeutically effective amount of a compound of the invention or a pharmaceutical composition thereof. The metabolic diseases and metabolic-related disorders are selected from, but not limited to, hyperlipidemia, type 1 diabetes, type 2 diabetes mellitus, idiopathic type 1 diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.

In an embodiment of the invention are therapeutic benefits of GPR119 activity modulators derived from increasing levels of GIP and PPY. For example, neuroprotection, learning and memory, seizures and peripheral neuropathy.

GLP-1 and GLP-1 receptor agonists have been shown to be effective for treatment of neurodegenerative diseases and other neurological disorders. GLP-1 and exendin-4 have been shown to stimulate neurite outgrowth and enhance cell survival after growth factor withdrawal in PC12 cells. In a rodent model of neurodegeneration, GLP-1 and exendin-4 restore cholinergic marker activity in the basal forebrain. Central infusion of GLP-1 and exendin-4 also reduce the levels of amyloid-13 peptide in mice and decrease amyloid precursor protein amount in cultured PC12 cells. GLP-1 receptor agonists have been shown to enhance learning in rats and the GLP-1 receptor knockout mice show deficiencies in learning behavior. The knockout mice also exhibit increased susceptibility to kainate-induced seizures which can be prevented by administration of GLP-1 receptor agonists. GLP-1 and exendin-4 has also been shown to be effective in treating pyridoxine-induced peripheral nerve degeneration, an experimental model of peripheral sensory neuropathy.

Glucose-dependent insulinotropic polypeptide (GIP) has also been shown to have effects on proliferation of hippocampal progenitor cells and in enhancing sensorimotor coordination and memory recognition.

In an embodiment of the invention are therapeutic benefits of GPR119 activity modulators. For example, GLP-2 and short bowel syndrome (SBS). Several studies in animals and from clinical trials have shown that GLP-2 is a trophic hormone that plays an important role in intestinal adaptation. Its role in regulation of cell proliferation, apoptosis, and nutrient absorption has been well documented. Short bowel syndrome is characterized by malabsorption of nutrients, water and vitamins as a result of disease or surgical removal of parts of the small intestine (e.g. Crohn's disease). Therapies that improve intestinal adaptation are thought to be beneficial in treatment of this disease. In fact, phase II studies in SBS patients have shown that teduglutide, a GLP-2 analog, modestly increased fluid and nutrient absorption.

In an embodiment of the invention are therapeutic benefits of GPR119 activity modulators derived from increasing levels of GIP and PPY. For example, GLP-1, GIP and osteoporosis. GLP-1 has been shown to increase calcitonin and calcitonin related gene peptide (CGRP) secretion and expression in a murine C-cell line (CA-77). Calcitonin inhibits bone resorption by osteoclasts and promotes mineralization of skeletal bone. Osteoporosis is a disease that is characterized by reduced bone mineral density and thus GLP-1 induced increase in calcitonin might be therapeutically beneficial.

GIP has been reported to be involved in upregulation of markers of new bone formation in osteoblasts including collagen type I mRNA and in increasing bone mineral density. Like GLP-1, GIP has also been shown to inhibit bone resorption.

In an embodiment of the invention are therapeutic benefits of GPR119 activity modulators derived from increasing levels of GIP and PPY. For example, PPY and gastric emptying. GPR119 located on the pancreatic polypeptide (PP) cells of the islets has been implicated in the secretion of PPY. PPY has been reported to have profound effects on various physiological processes including modulation of gastric emptying and gastrointestinal motility. These effects slow down the digestive process and nutrient uptake and thereby prevent the postprandial elevation of blood glucose. PPY can suppress food intake by changing the expression of hypothalamic feeding-regulatory peptides. PP-overexpressing mice exhibited the thin phenotype with decreased food intake and gastric emptying rate.

In accordance with the foregoing, the present invention further provides a method for preventing or ameliorating the symptamology of any of the diseases or disorders described above in a subject in need thereof, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

Compounds of the invention can be administered in therapeutically effective amounts in combination with one or more therapeutic agents (pharmaceutical combinations).

For example, synergistic effects can occur with other anti-obesity agents, anorectic agents, appetite suppressant and related agents. Diet and/or exercise can also have synergistic effects. Anti-obesity agents include, but are not limited to, apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agents, β3 adrenergic receptor agonists, dopamine agonists (for example, bromocriptine), melanocyte-stimulating hormone receptor analogs, cannabinoid 1 receptor antagonists [for example, compounds described in WO2006/047516), melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Where compounds of the invention are administered in conjunction with other therapies, dosages of the co-administered compounds will of course vary depending on the type of co-drug employed, on the specific drug employed, on the condition being treated and so forth.

A combined preparation or pharmaceutical composition can comprise a compound of the invention as defined above or a pharmaceutical acceptable salt thereof and at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPAR gamma agonist e.g. GI-262570; Diacylglycerol acetyltransferase (DGAT) inhibitors such as those disclosed in WO 2005044250, WO 2005013907, WO 2004094618 and WO 2004047755;

b) hypolipidemic agents such as 3-hydroxy-3-methyl-glutaryl coenzyme A (HMG-CoA) reductase inhibitors, e.g., lovastatin and related compounds such as those disclosed in U.S. Pat. No. 4,231,938, pitavastatin, simvastatin and related compounds such as those disclosed in U.S. Pat. Nos. 4,448,784 and 4,450,171, pravastatin and related compounds such as those disclosed in U.S. Pat. No. 4,346,227, cerivastatin, mevastatin and related compounds such as those disclosed in U.S. Pat. No. 3,983,140, velostatin, fluvastatin, dalvastatin, atorvastatin, rosuvastatin and related statin compounds disclosed in U.S. Pat. No. 5,753,675, rivastatin, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322. In addition, phosphinic acid compounds useful in inhibiting HMG CoA reductase suitable for use herein are disclosed in GB 2205837; squalene synthase inhibitors; FXR (farnesoid X receptor) and LXR (liver X receptor) ligands; cholestyramine; fibrates; nicotinic acid and aspirin;

c) an anti-obesity agent or appetite regulating agent such as a CB1 activity modulator, melanocortin receptor (MC4R) agonists, melanin-concentrating hormone receptor (MCHR) antagonists, growth hormone secretagogue receptor (GHSR) antagonists, galanin receptor modulators, orexin antagonists, CCK agonists, GLP-1 agonists, and other Pre-proglucagon-derived peptides; NPY1 or NPY5 antagonist, NPY2 and NPY4 modulators, corticotropin releasing factor agonists, histamine receptor-3 (H3) modulators, aP2 inhibitors, PPAR gamma modulators, PPAR delta modulators, acetyl-CoA carboxylase (ACC) inhibitors, 11-β-HSD-1 inhibitors, adinopectin receptor modulators; beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a thyroid receptor beta modulator, such as a thyroid receptor ligand as disclosed in WO 97/21993 (U. Cal SF), WO 99/00353 (KaroBio) and GB98/284425 (KaroBio), a SCD-1 inhibitor as disclosed in WO2005011655, a lipase inhibitor, such as orlistat or ATL-962 (Alizyme), serotonin receptor agonists, (e.g., BVT-933 (Biovitrum)), monoamine reuptake inhibitors or releasing agents, such as fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine or mazindol, anorectic agents such as topiramate (Johnson & Johnson), CNTF (ciliary neurotrophic factor)/Axokine® (Regeneron), BDNF (brain-derived neurotrophic factor), leptin and leptin receptor modulators, phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine;

d) anti-hypertensive agents such as loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; beta-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; aldosterone synthase inhibitors; and dual ET/AII antagonist such as those disclosed in WO 00/01389.

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, eplerenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

n) an agent for treating tobacco abuse, e.g., nicotine receptor partial agonists, bupropion hypochloride (also known under the tradename Zyban®) and nicotine replacement therapies;

o) an agent for treating erectile dysfunction, e.g., dopaminergic agents, such as apomorphine), ADD/ADHD agents (e.g., Ritalin®, Strattera®, Concerta® and Adderall®);

p) an agent for treating alcoholism, such as opioid antagonists (e.g., naltrexone (also known under the tradename ReVia®) and nalmefene), disulfiram (also known under the tradename Antabuse®), and acamprosate (also known under the tradename Campral®)). In addition, agents for reducing alcohol withdrawal symptoms may also be co-administered, such as benzodiazepines, beta-blockers, clonidine, carbamazepine, pregabalin, and gabapentin (Neurontin®);

q) other agents that are useful including anti-inflammatory agents (e.g., COX-2 inhibitors); antidepressants (e.g., fluoxetine hydrochloride (Prozac®)); cognitive improvement agents (e.g., donepezil hydrochloride (Aircept®) and other acetylcholinesterase inhibitors); neuroprotective agents (e.g., memantine); antipsychotic medications (e.g., ziprasidone (Geodon®), risperidone (Risperdal®), and olanzapine (Zyprexa®));

or, in each case a pharmaceutically acceptable salt thereof; and optionally a pharmaceutically acceptable carrier.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

In the following schemes, several methods of preparing the compounds of the present invention are illustrative. One of skill in the art will appreciate that these methods are representative, and in no way inclusive of all methods for preparing the compounds of the present invention. The radicals in the schemes are as described in Formula I.

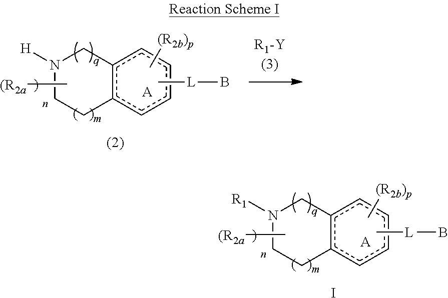

A compound of Formula I can be prepared by reacting a compound of formula 2 with a compound of formula 3 (Y=leaving group such as Cl, OMs, and the like) in the presence of a suitable solvent (for example, methylene chloride, and the like) and a suitable base (for example, pyridine, triethylamine, and the like). The reaction proceeds at a temperature of about 0° C. to about 50° C. and can take up to 24 h to complete.

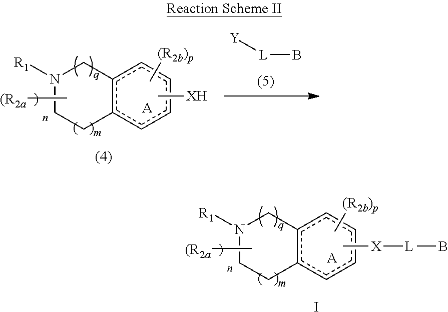

A compound of Formula I can be prepared by reacting a compound of formula 4 (XH=nucleophile such as OH, NHR and the like) with a compound of formula 5 (Y=leaving group such as Cl, OMs, and the like), in the presence of a suitable solvent (for example, acetonitrile, dimethylformamide, and the like) and a suitable base (for example, pyridine, triethylamine, Cs₂CO₃ and the like). The reaction proceeds at a temperature of about 0° C. to about 120° C. and can take up to 24 h to complete.

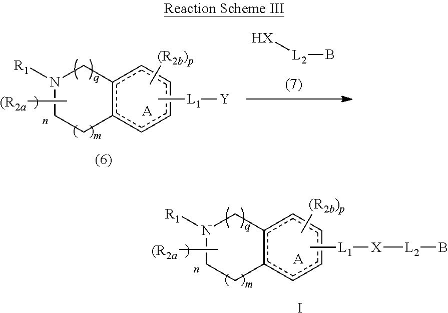

A compound of Formula I can be prepared by reacting a compound of formula 6 (Y=leaving group such as Cl, OMs, and the like) with a compound of formula 7 (XH=nucleophile such as OH, NHR and the like), in the presence of a suitable solvent (for example, tetrahydrofuran, dimethylformamide, and the like) and a suitable base (for example, NaH and the like). The reaction proceeds at a temperature of about 0° C. to about 50° C. and can take up to 24 h to complete.

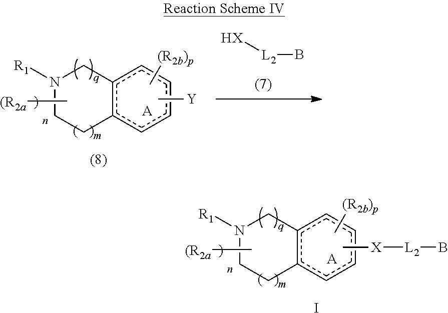

A compound of Formula I can be prepared by reacting a compound of formula 8 (Y=leaving group such as Cl, OMe, Ms, and the like) with a compound of formula 7 (XH=nucleophile such as OH, NHR and the like), neat or in the presence of a suitable solvent (for example, dimethylsulfoxide, THF, DMF, and the like) and a suitable base (for example, NaH, KHMDS, (ⁱPr)₂NEt, and the like). The reaction proceeds at a temperature of about 25° C. to about 200° C. and can take up to 24 h to complete.

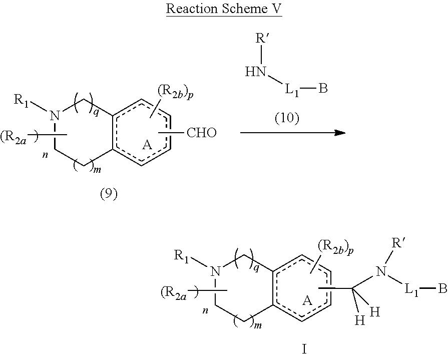

A compound of Formula I can be prepared by reacting an aldehyde of formula 9 with an amine of formula 10 in the presence of a suitable solvent (for example, tetrahydrofuran, and the like), a suitable reductant (sodiumtriacetoxyborohydride and the like) and a suitable acid (for example, acetic acid, and the like). The reaction proceeds at a temperature of about 0° C. to about 50° C. and can take up to 24 h to complete.

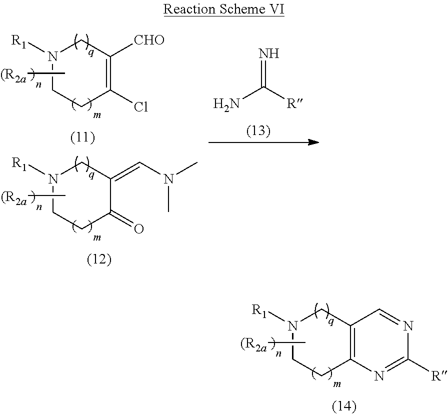

A compound of formula 14 can be prepared by reacting a compound of formula 11 or formula 12 with a compound of formula 13 in the presence of a suitable solvent (for example, dimethylformamide, ethanol, and the like), and optionally a suitable base (for example, triethylamine, potassiumacetate, and the like) or acid (for example, acetic acid, hydrochloric acid, and the like). The reaction proceeds at a temperature of about 50° C. to about 150° C. and can take up to 48 h to complete.

Detailed descriptions of the synthesis of compounds of the Invention are given in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be prepared conveniently, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction schemes I, II, III, IV, V & VI; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following Examples that illustrate the preparation of compounds of the invention and their intermediates.

### Intermediate 4: 6-Benzyl-5,6,7,8-tetrahydro-2,6-naphthyridin-1(2H)-one

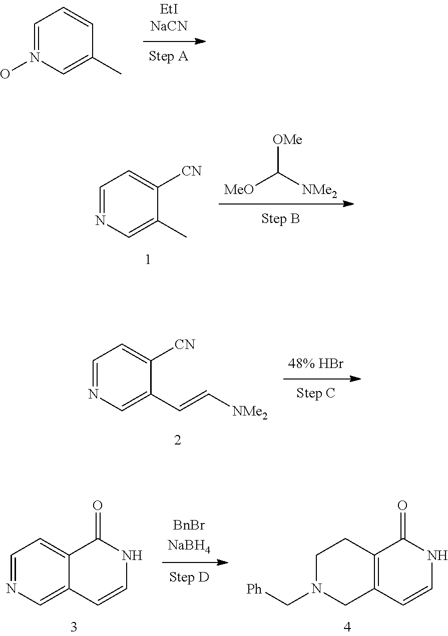

Step A: To a solution of 3-methylpyridine-N-oxide (240 g, 2.2 mol) in dichloromethane (4 L) is added ethyl iodide (530 mL, 6.6 mol). The mixture is stirred at reflux overnight. Then the suspension is cooled. The resulting precipitate is collected by filtration and washed with diethyl ether (500 mL) to give a white solid. The solid is dissolved in water (2.4 L) and warmed to 50° C. A solution of sodium cyanide (200 g, 4 mol) in water (600 mL) is slowly added over 1 h, keeping the internal temperature below 60° C. The reaction mixture is stirred at 55° C. for another 1 h. The reaction mixture is extracted with diethyl ether (3×1.5 L). The combined extracts are dried over MgSO₄ and concentrated to yield 4-cyano-3-methylpyridine 1 as a brown oil: ¹H NMR (400 MHz, CDCl₃) δ=8.66 (s, 1H), 8.58 (dd, J=6.8, 1.0 Hz, 1H), 7.46 (d, J=6.8 Hz, 1H), 2.54 (s, 3H).

Step B: To a solution of 4-cyano-3-methylpyridine 1 (123 g, 1.0 mol) in N,N-dimethylformamide (800 mL) is added N,N-dimethylformamide dimethyl acetal (800 mL). The mixture is heated at reflux for 18 h. After cooling and concentration in vacuo, the residue is dissolved in dichloromethane (400 mL) and precipitated with n-pentane. Filtration and washing with n-pentane, followed by drying under high vacuum, yielded 3-[(E)-2-(dimethylamino)ethenyl]-4-cyanopyridine 2 as a light-green solid: ¹H NMR (400 MHz, CDCl₃) δ=8.69 (s, 1H), 8.13 (d, J=6.8 Hz, 1H), 7.23 (dd, J=6.8, 1.0 Hz, 1H), 7.16 (d, J=17.6 Hz, 1H), 5.21 (d, J=17.6 Hz, 1H), 2.96 (s, 6H).

Step C: To a solution of 3-[(E)-2-(dimethylamino)ethenyl]-4-cyanopyridine 2 (70 g, 0.4 mol) in ethanol (700 mL) is added 48% hydrobromic acid (700 mL) over 1 h. The mixture is heated to reflux for 18 h. Filtration of the cooled mixture and washing with ethanol, followed by drying under high vacuum, yielded [2,6]-naphthyridin-1-(2H)-one hydrobromide 3 as a yellow solid: ¹H-NMR (400 MHz, CDCl₃) δ=11.7 (bs, 1H), 9.05 (s, 1H), 8.60 (d, J=6.8 Hz, 1H), 7.96 (d, J=7.0 Hz, 1H), 7.31 (d, J=9.6 Hz, 1H), 6.66 (d, J=9.2 Hz, 1H).

Step D: [2,6]-Naphthyridin-1-(2H)-one hydrobromide 3 (20 g, 88 mmol) is suspended in acetonitrile (500 mL) under nitrogen. Benzyl bromide (24.4 ml, 121 mmol) is added and the mixture is heated to reflux for 2 h, then concentrated in vacuo. The crude product is dissolved in ethanol (500 mL) and cooled to 0° C. Sodium borohydride (25.9 g, 685 mmol) is added portionwise over 30 min. The mixture is stirred at 0° C. for 1 h, then at rt for another 16 h. The reaction mixture is cooled to 0° C. again and 6 M hydrochloric acid (200 mL) is added dropwise over 30 min, then stirred at rt for 90 min. The resulting precipitate is filtered off, and the aqueous filtrate is basified with 2M sodium hydroxide (1 L). Extraction with ethyl acetate (250 mL), precipitation with cyclohexane, followed by filtration and drying under high vacuum, yielded 6-benzyl-5,6,7,8-tetrahydro-2,6-naphthyridin-1(2H)-one 4 as a tan solid: ¹H NMR (400 MHz, DMSO-d₆) δ=11.1 (bs, 1H), 7.21-7.25 (m, 5H), 7.10 (d, J=8.8 Hz, 1H), 5.86 (d, J=8.8 Hz, 1H), 3.60 (s, 2H), 3.29 (s, 2H), 2.59 (t, J=8.0 Hz, 2H), 2.37 (t, J=8.0 Hz, 2H); MS calcd. for C₁₆H₁₇N₂O [M+H⁺] 241.1. found 241.5.

### Intermediates 6 (Isopropyl 4-(3-(6-benzyl-5,6,7,8-tetrahydro-1-oxo-2,6-naphthyridin-2(5H)-yl)propyl)piperidine-1-carboxylate) and 7 (isopropyl 4-(3-(2-benzyl-1,2,3,4-tetrahydro-2,6-naphthyridin-5-yloxy)propyl)piperidine-1-carboxylate)

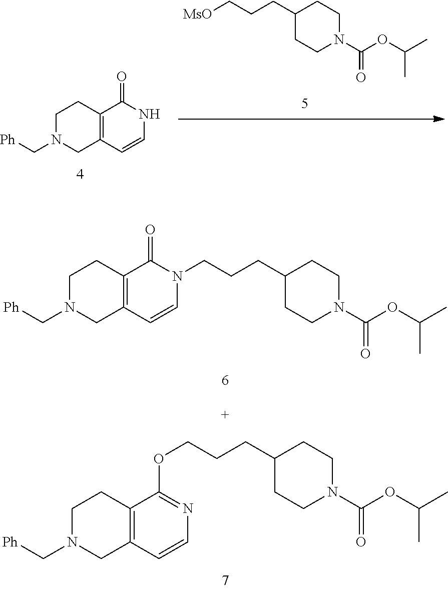

6-Benzyl-5,6,7,8-tetrahydro-2,6-naphthyridin-1(2H)-one 4 (34.8 mg, 0.15 mmol) and isopropyl 4-(3-(methylsulfonyloxy)propyl)piperidine-1-carboxylate 5 (53.8 mg, 0.18 mmol, made similarly to Intermediate 34 below) are dissolved in acetonitrile (2.5 mL). Powdered cesium carbonate (0.10 g, 0.3 mmol) is added and the resulting suspension is stirred at 65° C. overnight. Cooling, filtration, and separation of the regioisomers by reverse-phase HPLC (H₂O/MeCN gradient) yields isopropyl 4-(3-(6-benzyl-5,6,7,8-tetrahydro-1-oxo-2,6-naphthyridin-2(5H)-yl)propyl)piperidine-1-carboxylate 6 [MS calcd. for C₂₇H₃₈N₃O₃ [M+H⁺] 452.2. found 452.3] and isopropyl 4-(3-(2-benzyl-1,2,3,4-tetrahydro-2,6-naphthyridin-5-yloxy)propyl)piperidine-1-carboxylate 7 [MS calcd. for C₂₇H₃₈N₃O₃ [M+H⁺] 452.2. found 452.3].

### Example A1

### Isopropyl 4-(3-(1,2,3,4-tetrahydro-2-methanesulfonyl-5-oxo-2,6-naphthyridin-6(5H)-yl)propyl)piperidine-1-carboxylate

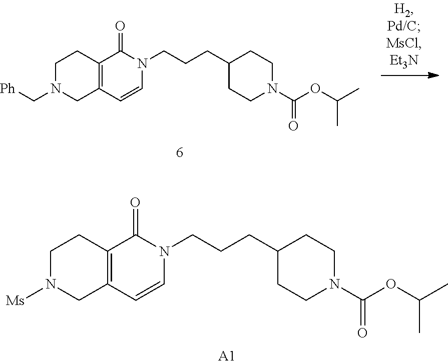

Isopropyl 4-(3-(6-benzyl-5,6,7,8-tetrahydro-1-oxo-2,6-naphthyridin-2(5H)-yl)propyl)piperidine-1-carboxylate 6 (40 mg, 0.075 mmol) is dissolved in a 1:1 mixture of ethyl acetate and absolute ethanol (3 mL). The solution is subjected to 1 atm hydrogen pressure using the H-Cube® at 70° C., with 10% palladium black on charcoal as catalyst. The solution is concentrated in vacuo. The remainder is dissolved in dichloromethane (2.5 mL), treated with triethylamine (50 μL, 0.36 mmol) and methanesulfonylchloride (10 μL, 0.13 mmol) and stirred for 30 min at rt. Concentration and purification by reverse-phase HPLC (H₂O/MeCN gradient) yields isopropyl 4-(3-(1,2,3,4-tetrahydro-2-methanesulfonyl-5-oxo-2,6-naphthyridin-6 (5H)-yl)propyl)piperidine-1-carboxylate A1 as a white solid. ¹H NMR (400 MHz, CDCl₃) δ=7.19 (d, J=7.0 Hz, 1H), 6.05 (d, J=7.0 Hz, 1H), 4.90 (septet, J=6.2 Hz, 2H), 4.26 (s, 2H), 4.11 (d, J=11.2 Hz, 2H), 3.94 (t, J=7.4 Hz, 2H), 3.53 (d, J=5.9 Hz, 2H), 2.87 (s, 3H), 2.79 (m, 2H), 2.70 (m, 2H), 1.77 (m, 2H), 1.66 (d, J=12.8 Hz, 2H), 1.43 (m, 1H), 1.30 (m, 2H), 1.23 (d, J=6.2 Hz, 6H), 1.08 (ddd, J=4.5, 11.9, 12.9 Hz, 2H); MS calcd. for C₂₁H₃₄N₃O₅S [M+H⁺] 440.2. found 440.1.

### Examples A2

### [Isopropyl 4-(3-(1,2,3,4-tetrahydro-2-methanesulfonyl-2,6-naphthyridin-5-yloxy)propyl)piperidine-1-carboxylate], and A3 [isopropyl 4-(3-(1,2,3,4,4a,7,8,8a-octahydro-2-methanesulfonyl-2,6-naphthyridin-5-yloxy)propyl)piperidine-1-carboxylate]

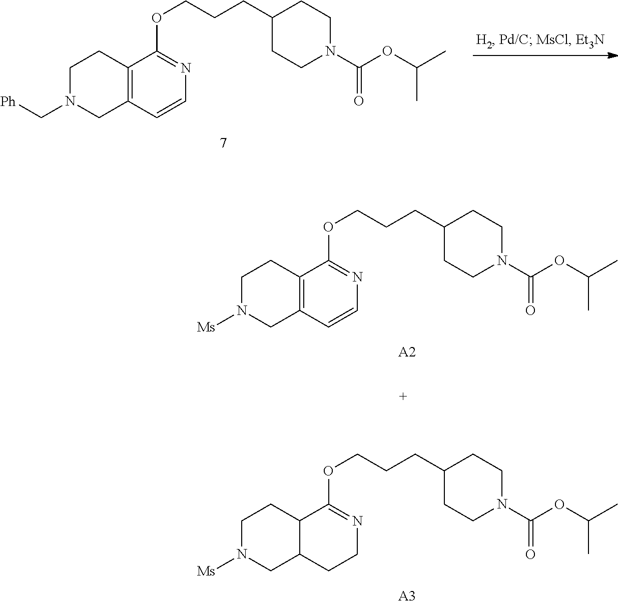

Isopropyl 4-(3-(2-benzyl-1,2,3,4-tetrahydro-2,6-naphthyridin-5-yloxy)propyl)piperidine-1-carboxylate 7 (40 mg, 0.075 mmol) is dissolved in a 1:1 mixture of ethyl acetate and absolute ethanol (3 mL). The solution is subjected to 1 atm hydrogen using the H-Cube® at 70° C., with 10% palladium black on charcoal as catalyst. The solution is concentrated in vacuo. The remainder is dissolved in dichloromethane (2.5 mL), treated with triethylamine (50 μL, 0.36 mmol) and methanesulfonylchloride (10 μL, 0.13 mmol) and stirred for 30 min at rt. Concentration and purification by reverse-phase HPLC (H₂O/MeCN gradient) yields isopropyl 4-(3-(1,2,3,4-tetrahydro-2-methanesulfonyl-2,6-naphthyridin-5-yloxy)propyl)piperidine-1-carboxylate A2 and isopropyl 4-(3-(1,2,3,4,4a,7,8,8a-octahydro-2-methanesulfonyl-2,6-naphthyridin-5-yloxy)propyl)piperidine-1-carboxylate A3 as white solids. A2: ¹H NMR (400 MHz, CDCl₃) δ=8.05 (d, J=5.6 Hz, 1H), 6.75 (d, J=5.6 Hz, 1H), 4.91 (septet, J=6.2 Hz, 2H), 4.43 (s, 2H), 4.37 (t, J=6.6 Hz, 2H), 3.59 (t, J=6.0 Hz, 2H), 2.89 (s, 3H), 2.84 (t, J=6.0 Hz, 2H), 2.73 (t, J=12.4 Hz, 2H), 1.84 (m, 2H), 1.70 (d, J=13.0 Hz, 2H), 1.50 (m, 1H), 1.41 (m, 2H), 1.24 (d, J=6.2 Hz, 6H), 1.12 (m, 2H), 0.86 (m, 2H); MS calcd. for C₂₁H₃₄N₃O₅S [M+H⁺] 440.2. found 440.2; A3: MS calcd. for C₂₁H₃₈N₃O₅S [M+H⁺] 444.2. found 444.2.

By repeating the procedure described in the above examples A1-A3, using appropriate starting materials, the following compounds of Formula I, as identified in Table 1, are obtained.

TABLE 1

| Ex. # | Structure | NMR and/or ESMS |
|---|---|---|
| A4 | 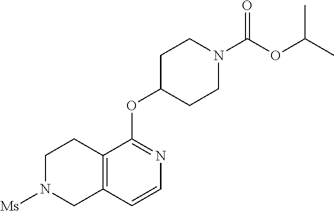 | MS calcd. for C₁₈H₂₈N₃O₅S [M + H]⁺: 398.2, found: 398.2. |
| A5 | 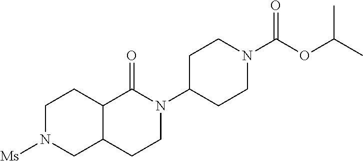 | MS calcd. for C₁₈H₃₂N₃O₅S [M + H]⁺: 402.2, found: 402.2. |
| A6 | 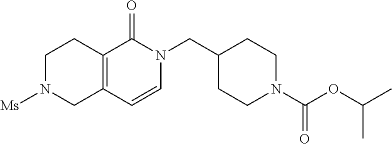 | MS calcd. for C₁₉H₃₀N₃O₅S [M + H]⁺: 412.2, found: 412.1. |
| A7 | 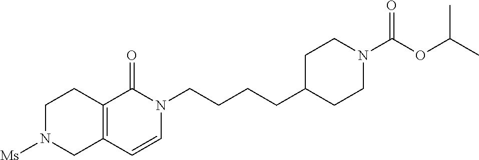 | MS calcd. for C₂₂H₃₆N₃O₅S [M + H]⁺: 454.3, found: 454.2. |
| A8 | 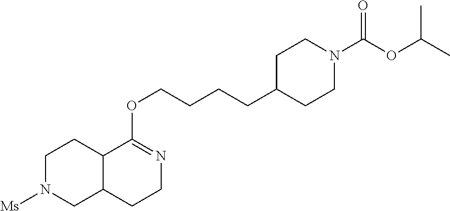 | MS calcd. for C₂₂H₄₀N₃O₅S [M + H]⁺: 458.3, found: 458.2. |
| A9 | 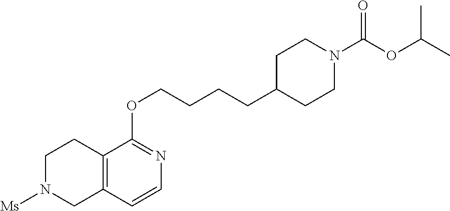 | MS calcd. for C₂₂H₃₆N₃O₅S [M + H]⁺: 454.3, found: 454.2. |

### Intermediate 10: 6-(Methylsulfonyl)-5,6,7,8-tetrahydropyrido[4,3-d]pyrimidine-2-carbaldehyde

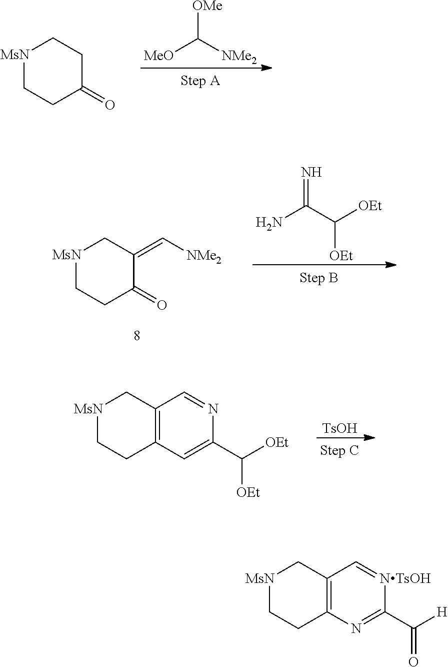

Step A: To a solution of 1-(methylsulfonyl)piperidin-4-one (20 g, 113 mmol) in DMF (17 mL) is added N,N-dimethylformamide dimethyl acetal (16.6 mL, 124 mmol). The mixture is stirred at 90° C. under nitrogen for 18 h. The precipitate is collected and washed with cold Et₂O to afford 3-((dimethylamino)methylene)-1-(methylsulfonyl)piperidin-4-one 8 as a light yellow solid. The filtrate is evaporated and minimal ethyl acetate is added. After stirring for 15 min the solid is collected and washed with cold Et₂O to afford additional product 8. The combined product 8 is used in the next step without further purification: ¹H NMR (400 MHz, CDCl₃) δ=7.58 (br s, 1H), 4.48 (s, 2H), 3.58 (t, J=6.4 Hz, 2H), 3.14 (s, 6H), 2.89 (s, 3H), 2.58 (t, J=6.4 Hz, 2H); MS calcd. for C₉H₁₇N₂O₃S [M+H⁺] 233.0. found 233.0.

Step B: To a 250 mL round-bottomed flask containing EtOH (130 mL) is added Na metal (738 mg, 32.1 mmol) and the mixture is stirred until complete dissolution. To this solution are then added intermediate 8 (6.21 g, 26.7 mmol) and 2,2-diethoxyacetamidine (4.40, 30 mmol). The mixture is heated to 95° C. for 6 h. Ethyl acetate and sat. aq. NaHCO₃ are then added, the organic layer is separated, and the aqueous layer extracted with ethyl acetate (3×). The combined organics are dried (Na₂CO₃) and concentrated. The crude is purified by flash chromatography (100% ethyl acetate) to afford 2-(diethoxymethyl)-6-(methylsulfonyl)-5,6,7,8-tetrahydropyrido[4,3-d]pyrimidine 9 as a white solid. ¹H NMR (400 MHz, CDCl₃) δ=8.56 (s, 1H), 5.56 (s, 1H), 4.52 (s, 2H), 3.85-3.77 (m, 2H), 3.75-3.67 (m, 4H), 3.20 (t, J=6.0 Hz, 2H), 2.94 (s, 3H), 1.29 (t, J=7.2 Hz, 2H); MS calcd. for C₁₃H₂₂N₃O₄S [M+H⁺] 316.1. found 316.1.

Step C: To a solution of 9 (3.09 g, 9.8 mmol) in 2:1 acetone/water (39 mL) is added p-toluenesulfonic acid (560 mg, 2.94 mmol). The mixture is heated to 50° C. for 18 h. Additional p-toluenesulfonic acid (187 mg, 0.98 mmol) is added and stirring at 50° C. is continued for 6 h. The mixture is then concentrated, diluted with sat. aq. NaHCO₃ and extracted with ethyl acetate (5×). The organic phase is washed with brine, dried (Na₂SO₄) and concentrated. The crude material is purified by reverse-phase HPLC (H₂O/MeCN gradient) to afford 6-(methylsulfonyl)-5,6,7,8-tetrahydropyrido[4,3-d]pyrimidine-2-carbaldehyde 10 as a p-toluenesulfonic acid salt: ¹H NMR (400 MHz, CD₃OD) δ=8.74 (s, 1H), 7.71 (d, J=8.0 Hz, 2H), 7.24 (d, J=8.0 Hz, 2H), 5.59 (s, 1H), 4.58 (s, 2H), 3.70 (t, J=6.0 Hz, 2H), 3.18 (t, J=6.0 Hz, 2H), 2.99 (s, 3H), 2.38 (s, 3H); MS calcd. for C₉H₁₂N₃O₃S [M+H⁺] 242.0. found 241.9.

### Example B1

### tert-Butyl 4-(((6-(methylsulfonyl)-5,6,7,8-tetrahydropyrido[4,3-d]pyrimidin-2-yl)methylamino)methyl)piperidine-1-carboxylate

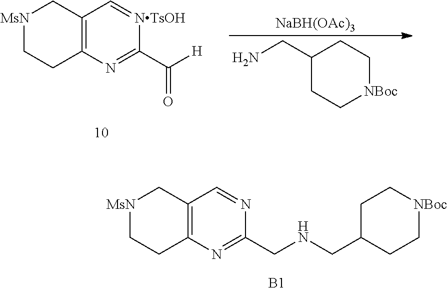

To a solution of 10 (40 mg, 0.09 mmol) in THF (0.5 mL) is added tert-butyl 4-(aminomethyl)piperidine-1-carboxylate (53 mg, 0.25 mmol) followed by NaBH(OAc)₃ (88 mg, 0.41 mmol) and acetic acid (11 μL, 0.19 mmol). The mixture is stirred at rt overnight, filtered, and purified by reverse phase HPLC (H₂O/MeCN gradient) to afford tert-butyl 4-(((6-(methylsulfonyl)-5,6,7,8-tetrahydropyrido[4,3-d]pyrimidin-2-yl)methylamino)methyl)piperidine-1-carboxylate B1: ¹H NMR (400 MHz, CD₃CN) δ=8.82 (br. s, 1H), 8.58 (s, 1H), 4.49 (s, 2H), 4.41 (s, 2H), 4.08 (br. d, J=12.4 Hz, 2H), 3.64 (t, J=6.0 Hz, 2H), 3.08-3.04 (m, 4H), 2.93 (s, 3H), 2.76 (m, 2H), 2.07-2.00 (m, 1H), 1.82-1.79 (m, 2H), 1.45 (s, 9H), 1.23-1.13 (m, 2H); MS calcd. for C₂₀H₃₄N₅O₄S [M+H⁺] 440.2. found 440.2.

By repeating the procedure described in the above example B1, using appropriate starting materials, the following compounds of Formula I, as identified in Table 2, are obtained.

TABLE 2

| Ex. # | Structure | NMR and/or ESMS |
|---|---|---|
| B2 | 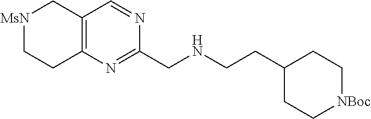 | ¹H NMR (400 MHz, CD₃CN) δ = 8.84 (br. s, 1H), 8.46 (s, 1H), 4.38 (s, 2H), 4.29 (s, 2H), 3.93 (br. d, J = 12.8 Hz, 2H), 3.53 (t, J = 6.0 Hz, 2H), 3.08 (t, J = 7.2 Hz, 2H), 2.95 (t, J = 6.0 Hz, 2H), 2.82 (s, 3H), 2.61 (m, 2H), 1.69-1.56 (m, 4H), 1.52-1.44 (m, 1H), 1.33 (s, 9H), 1.02-0.92 (m, 2H); MS calcd. for C₂₁H₃₆N₅O₄S [M + H]⁺: 454.2, found: 454.2. |
| B3 | 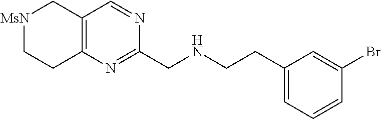 | ¹H NMR (400 MHz, CDCl₃) δ = 8.44 (s, 1H), 7.36- 7.33 (m, 2H), 7.17-7.15 (m, 2H), 4.47 (s, 2H), 4.04 (s, 2H), 3.66 (d, J = 6.0 Hz, 2H), 3.08 (t, J = 6.0 Hz, 2H), 2.96-2.92 (m, 2H), 2.91 (s, 3H), 2.86-2.83 (m, 2H); MS calcd. for C₁₇H₂₂BrN₄O₂S [M + H]⁺: 425.1, found: 425.0. |
| B4 | 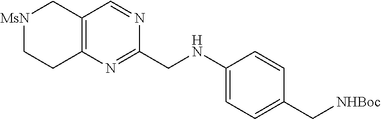 | ¹H NMR (400 MHz, CDCl₃) δ = 8.40 (s, 1H), 7.05 (d, J = 8.4 Hz, 2H), 6.62 (d, J = 8.4 Hz, 2H), 4.96 (br. s, 1H), 4.65 (br. s, 1H), 4.44 (s, 2H), 4.41 (s, 2H), 4.12 (d, J = 5.2 Hz, 2H), 3.60 (t, J = 6.0 Hz, 2H), 3.06 (t, J = 6.0 Hz, 2H), 2.85 (s, 3H), 1.38 (s, 9H); MS calcd. for C₂₁H₃₀N₅O₄S [M + H]⁺: 448.2, found: 448.1. |

### Intermediate 12: 2-(Bromomethyl)-6-(methylsulfonyl)-5,6,7,8-tetrahydropyrido[4,3-d]pyrimidine

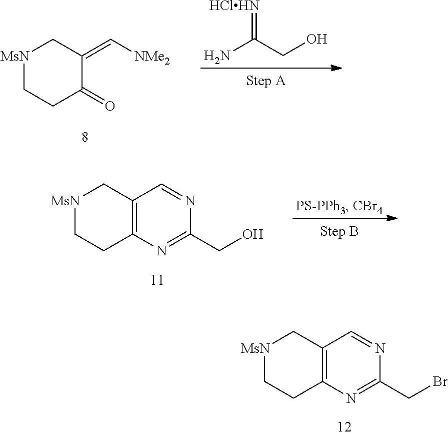

Step A: Hydroxyacetamidine hydrochloride (134 mg, 1.21 mmol) and intermediate 8 (250 mg, 1.08 mmol) are converted to (6-(methylsulfonyl)-5,6,7,8-tetrahydropyrido[4,3-d]pyrimidin-2-yl)methanol 11 following the same procedure as for the preparation of intermediate 9. ¹H NMR (400 MHz, CDCl₃) δ=8.42 (s, 1H), 4.73 (s, 2H), 4.42 (s, 2H), 3.61 (t, J=6.0 Hz, 2H), 3.06 (t, J=6.0 Hz, 2H), 3.02 (s, 1H), 2.86 (s, 3H); MS calcd. for C₉H₁₄N₃O₃S [M+H⁺] 244.0. found 244.0.

Step B: A mixture of 11 (200 mg, 0.82 mmol), polystyrene supported triphenylphosphine (2.23 mmol/g, 774 mg) and carbon tetrabromide (545 mg, 1.64 mmol) in dichloromethane (5 mL) is stirred at rt for 18 h. The solid is then filtered and washed with dichloromethane. Concentration of the filtrate afforded 2-(bromomethyl)-6-(methylsulfonyl)-5,6,7,8-tetrahydropyrido[4,3-d]pyrimidine 12. The crude is used in the next step without further purification. ¹H NMR (400 MHz, CDCl₃) δ=8.44 (s, 1H), 4.51 (s, 2H), 4.43 (s, 2H), 3.61 (t, J=6.0 Hz, 2H), 3.07 (t, J=6.0 Hz, 2H), 2.86 (s, 3H); MS calcd. for C₉H₁₃BrN₃O₂S [M+H⁺] 306.0. found 306.0.

### Intermediate 13: 1-Methylcyclopropyl 4-(2-hydroxyethyl)piperidine-1-carboxylate

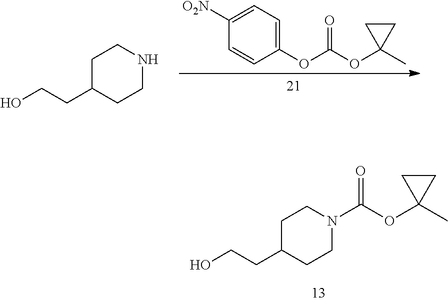

A solution of 4-piperidinethanol (163 mg, 1.26 mmol) and 1-methylcyclopropyl 4-nitrophenyl carbonate 21 (300 mg, 1.26 mmol) are dissolved in dichloromethane (6 mL). Triethylamine (0.21 mL, 1.52 mmol) is added and the reaction mixture is stirred at rt overnight. Then it is diluted with dichloromethane washed with 1M NaOH (4×). The organic phase is then washed with 1M HCl (1×) and brine (1×), dried (Na₂SO₄) and concentrated to afford 1-methylcyclopropyl 4-(2-hydroxyethyl)piperidine-1-carboxylate 13 (287 mg, quant.). The crude product is used in the next step without further purification. ¹H NMR (400 MHz, CDCl₃) δ=4.16-3.80 (m, 2H), 3.66-3.62 (m, 2H), 3.63 (t, J=12.8 Hz, 2H), 1.63-1.59 (m, 2H), 1.47 (s, 3H), 1.21 (m, 1H), 1.08-1.00 (m, 2H), 0.80-0.77 (m, 2H), 0.56-0.53 (m, 2H); MS calcd. for C₁₂H₂₂NO₃ [M+H⁺] 228.1. found 228.1.

### Example C1

### 1-Methylcyclopropyl 4-(2-((6-(methylsulfonyl)-5,6,7,8-tetrahydropyrido[4,3-d]pyrimidin-2-yl)methoxy)ethyl)piperidine-1-carboxylate

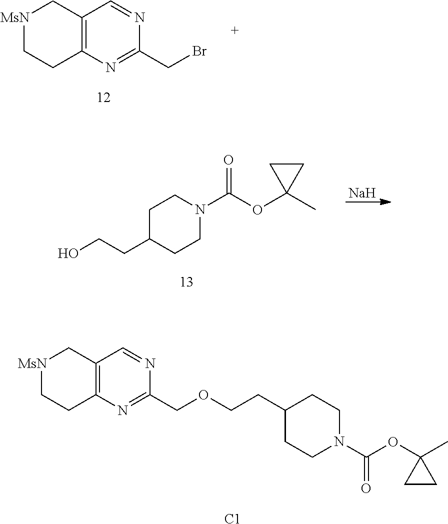

A solution of 13 (44.5 mg, 0.2 mmol) in THF (0.4 mL) is cooled to 0° C., then NaH (5.1 mg, 0.13 mmol) is added. The mixture is stirred for 30 min at this temperature. A solution of 12 (30 mg, 0.01 mmol) in THF (0.1 mL) is added and the mixture is stirred at 50° C. overnight. The reaction is quenched with sat. aq. NH₄Cl, filtered, washed with acetonitrile and purified by reverse phase HPLC to afford 1-methylcyclopropyl 4-(2-((6-(methylsulfonyl)-5,6,7,8-tetrahydropyrido[4,3-d]pyrimidin-2-yl)methoxy)ethyl)piperidine-1-carboxylate C3: ¹H NMR (400 MHz, CDCl₃) δ=8.45 (s, 1H), 4.89 (s, 2H), 4.35 (s, 2H), 4.00-3.77 (m, 2H), 3.51 (t, J=6.0 Hz, 4H), 2.92 (t, J=6.0 Hz, 2H), 2.80 (s, 3H), 2.63-2.53 (m, 2H), 1.59-1.53 (m, 2H), 1.52-1.46 (m, 1H), 1.47-1.41 (m, 2H), 1.39 (s, 3H), 0.98-0.88 (m, 2H), 0.71-0.68 (m, 2H), 0.51-0.48 (m, 2H); MS calcd. for C₂₁H₃₃N₄O₅S [M+H⁺] 453.2. found 453.2.

### Intermediate 16: 3-(1-(3-Isopropyl-1,2,4-oxadiazol-5-yl)piperidin-4-yl)propyl methanesulfonate

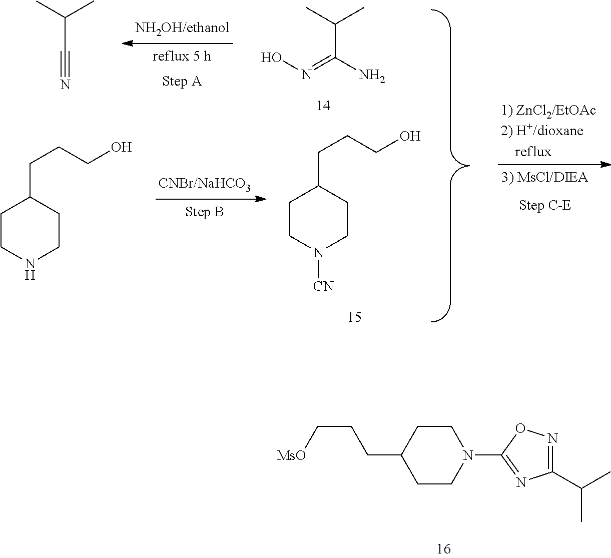

Step A: A solution of isobutyronitrile (13.82 g, 0.20 mol) and hydroxylamine (50% in water, 49 mL, 0.80 mol) in 95% ethanol is heated to reflux overnight, then concentrated in vacuo. The residual water is removed azeotropically with toluene to give N′-hydroxyisobutyrimidamide 14 as a light yellow solid: ¹H NMR (400 MHz, CDCl₃) δ=7.00 (br s, 1H), 4.52 (s, 2H), 2.45 (quint. J=5.4 Hz, 1H), 1.16 (d, J=5.4 Hz, 6H).

Step B: To a stirred suspension of sodium bicarbonate (2.80 g, 33.3 mmol) and 4-piperidinepropanol hydrochloride salt (2.00 g, 11.1 mmol) in water (1.5 mL) and CH₂Cl₂ (2 mL) is added a solution of cyanogen bromide (1.42 g, 13.4 mmol) in CH₂Cl₂ (3 mL) at 0° C. over a period of 1 h. The ice bath is removed and the reaction mixture is stirred at rt overnight. Then excess sodium carbonate (0.33 g) is added, the reaction mixture is diluted with CH₂Cl₂ (20 mL) and dried with 1.7 g of MgSO₄. The mixture is filtered, washed with CH₂Cl₂, and concentrated to give 4-(3-hydroxypropyl)piperidine-1-carbonitrile 15 as amber colored thick oil: ¹H NMR (400 MHz, CDCl₃) δ=3.64 (t, J=4.8 Hz, 2H), 3.42 (m, 2H), 2.99 (t, J=9.0 Hz, 2H), 1.73 (m, 2H), 1.55 (m, 2H), 1.49 (br s, 1H), 1.36-1.25 (m, 5H); MS calcd. for C₉H₁₇N₂O [M+H⁺]: 169.1. found: 169.0.

Step C: To a stirred solution of 4-(3-hydroxypropyl)piperidine-1-carbonitrile 15 (1.87 g, 11.1 mmol) and N′-hydroxyisobutyrimidamide 14 (1.70 g, 16.7 mmol) in EtOAc (40 mL) is slowly added ZnCl₂ (16.7 mL, 1N in ether). A precipitate formed during the addition and the reaction mixture is stirred at rt for 15 min. The solvent is decanted and the remainder is triturated with ether (40 mL) until a yellow suspension is obtained. The precipitate is collected by filtration, washed with ether (30 mL) and dried to give a yellow solid (5.25 g): MS calcd. for C₁₃H₂₇N₄O₂ [M+H]⁺: 271.2. found: 271.2.

Step D: To a suspension of the above solid (422 mg, approx 0.90 mmol) in dioxane (10 mL) is added HCl (4N, in dioxane, 0.45 mL). The mixture is stirred at 100° C. for 20 min. The reaction mixture is neutralized with 1N NaOH (4 mL) and concentrated. The off white residue is dried under high vacuum: MS calcd. for C₁₃H₂₄N₃O₂ [M+H]⁺: 254.2. found: 254.1.

Step E: The crude product (approx 0.90 mmol) obtained in step D is dissolved in CH₂Cl₂ (20 mL). DIEA (0.21 mL, 2.7 mmol) is added followed by addition of MeSO₂Cl (0.595 mL, 3.6 mmol) at 0° C. The reaction is stirred at rt overnight. The insoluble material is filtered off, washed with CH₂Cl₂ and the filtrate is concentrated. The remainder is purified by flash chromatography (SiO₂, EtOAc/hexanes gradient) to give 3-(1-(3-isopropyl-1,2,4-oxadiazol-5-yl)piperidin-4-yl)propyl methanesulfonate 16 as a light tan colored solid: ¹H NMR (400 MHz, CDCl₃) δ=4.23 (t, J=4.8 Hz, 2H), 4.13 (m, 2H), 3.02 (m, 2H), 3.01 (s, 3H), 2.88 (septet, J=5.1 Hz, 1H), 1.78 (m, 4H), 1.50 (m, 1H), 1.39 (m, 2H), 1.28 (d, J=5.1 Hz, 6H), 1.26 (m, 2H); MS calcd. for C₁₄H₂₆N₃O₄S [M+H]⁺: 332.2. found: 332.1.

### Intermediate 18: 6-(methylsulfonyl)-5,6,7,8-tetrahydropyrido[4,3-d]pyrimidin-2-ol

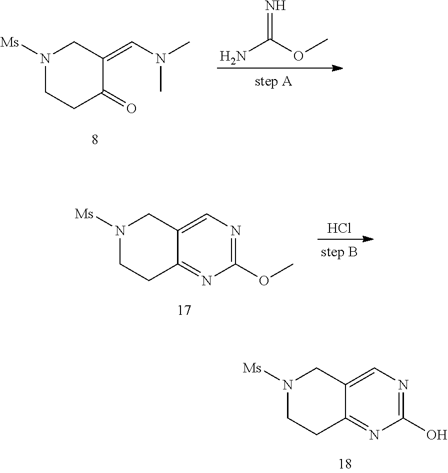

Step A: A mixture of 3-((dimethylamino)methylene)-1-(methylsulfonyl)-piperidin-4-one 8 (4.80 g, 20.6 mmol), O-methylisourea hydrochloride (3.43 g, 31 mmol) and TEA (5.7 mL, 41.2 mmol) in ethanol (100 mL) is stirred at 80° C. in a sealed tube overnight. The solvent is removed in vacuo. Saturated NaHCO₃ (25 mL) is added and the mixture is extracted with EtOAc (3×50 mL). The organic layer is washed with brine (20 mL), dried over MgSO₄, and concentrated to give a light yellow solid. The solid is suspended in EtOAc (about 10 mL) and stirred at rt overnight. An off white solid is collected by filtration, washed with ether and dried to give 2-methoxy-6-(methylsulfonyl)-5,6,7,8-tetrahydropyrido[4,3-d]pyrimidine 17: ¹H NMR (400 MHz, CD₃CN) δ=8.31 (s, 1H), 4.35 (s, 2H), 3.92 (s, 3H), 3.56 (t, J=4.5 Hz, 2H), 2.93 (t, J=4.5 Hz, 2H), 2.87 (s, 3H); MS calcd. for C₉H₁₄N₃O₃S [M+H]⁺: 244.1. found: 243.9.

Step B: 2-Methoxy-6-(methylsulfonyl)-5,6,7,8-tetrahydropyrido[4,3-d]pyrimidine 17 (3.67 g, 15.1 mmol) is dissolved in MeOH (5 mL) and stirred in conc HCl (15 mL) at 80° C. for 3 h. After concentration the residue is coevaporated repeatedly with MeOH, then dried in vacuo to give 6-(methylsulfonyl)-5,6,7,8-tetrahydropyrido[4,3-d]pyrimidin-2-ol 18 as a yellowish solid (3.68 g): ¹H NMR (400 MHz, d₆-dmso) δ=8.15 (s, 1H), 4.15 (s, 2H), 3.43 (t, J=6.0 Hz, 2H), 2.96 (s, 3H), 2.75 (t, J=6.0 Hz, 2H); MS calcd. for C₈H₁₂N₃O₃S [M+H]⁺: 230.1. found: 230.0.

### Example D1

### 3-Isopropyl-5-(4-(3-(6-(methylsulfonyl)-5,6,7,8-tetrahydropyrido[4,3-d]pyrimidin-2-yloxy)propyl)piperidin-1-yl)-1,2,4-oxadiazole

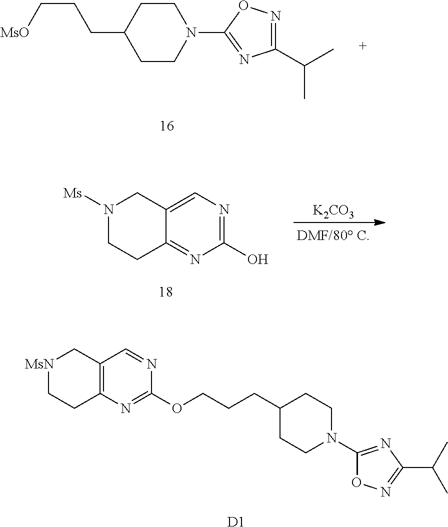

To a solution of 6-(methylsulfonyl)-5,6,7,8-tetrahydropyrido[4,3-d]pyrimidin-2-ol 18 (68 mg, 0.25 mmol) in DMF (3 mL) is added potassium carbonate (204 mg, 1.5 mmol). After stirring at rt for 5 min, 3-(1-(3-isopropyl-1,2,4-oxadiazol-5-yl)piperidin-4-yl)propyl methanesulfonate 16 (200 mg, 0.6 mmol) is added to the reaction. The reaction mixture is stirred in sealed vial at 80° C. for 5 h. The mixture is concentrated in vacuo, and the residue is purified by reverse-phase HPLC (H₂O/MeCN gradient) to give 3-isopropyl-5-(4-(3-(6-(methylsulfonyl)-5,6,7,8-tetrahydropyrido[4,3-d]pyrimidin-2-yloxy)propyl)piperidin-1-yl)-1,2,4-oxadiazole D1 as a white powder: ¹H NMR (400 MHz, CD₃CN) δ=8.29 (s, 1H), 4.35 (s, 2H), 4.29 (t, J=4.8 Hz, 2H), 4.02 (m, 2H), 3.56 (t, J=4.5 Hz, 2H), 3.04 (dt, J=2.1, 9.6 Hz, 2H), 2.92 (t, J=4.5 Hz, 2H), 2.87 (s, 3H), 2.81 (quint., J=5.1 Hz, 1H), 1.79 (m, 4H), 1.54 (m, 1H), 1.40 (m, 2H), 1.21 (d, J=5.4 Hz, 6H), 1.20 (m, 2H); MS calcd. for C₂₁H₃₃N₆O₄S [M+H]⁺: 465.2. found: 465.2.

### Intermediate 19: Tetrakis(cyclohexyloxy)titanium 19

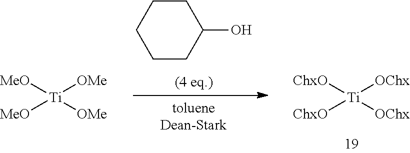

The published routes to acyl donors of cyclopropylmethyl alcohol 20 are inadequate since they result in the contamination of the product with isopropanol from the titanium isopropoxide catalyst. The titanium cyclohexyloxy catalyst 19 is prepared instead: A 25 mL flask is charged with Ti(OMe)₄ (3.25 g, 18.9 mmol) and cyclohexanol (7.57 g, 75.6 mL) and toluene (15 mL). The system is heated to 140° C. with a Dean-Stark trap until no more MeOH is generated, then the toluene is removed. This cycle is repeated twice and the remainder is used without further purification.

### Intermediate 21: 1-Methylcyclopropyl 4-nitrophenyl carbonate

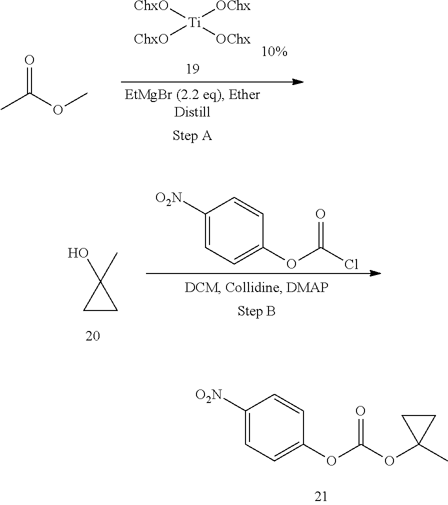

Step A: A 2 L flask is treated with 500 mL of ether, the above catalyst 19 and methyl acetate (14 g, 0.189 mol). To this solution is added a 3 M solution of ethyl magnesium bromide in diethyl ether (139 mL, 0.416 mol) over the course of 1.5 h. The temperature is kept constant by suspending the flask in a water bath. After the addition is complete, the reaction mixture is stirred for an additional 15 min and then quenched into an ice cold 10% solution of H₂SO₄ in water (1.6 L). The organic phase is separated and the aqueous phase is extracted twice more with 250 mL portions of ether. The combined organics are extracted with 50 mL of saturated aqueous sodium hydrogencarbonate, dried over MgSO₄, filtered and distilled. The ether is removed without vacuum at 65° C. and the residue is distilled through a short path distillation apparatus. The desired 1-methylcyclopropanol 20 boils at roughly 100° C. Once the product fraction (5.0 g) is collected, it is examined by NMR and the rough purity is 50% with the rest of the material being toluene, ether and methyl ethyl ketone. This material is used in the next step without further purification.

Step B: An ice cold solution of 4-nitrophenyl chloroformate (6.99 g, 34 mmol) in dichloromethane (50 mL) is treated with a solution of 20 from the previous step along with DMAP (424 mg, 3.47 mmol) in 2,4,6-collidine (25 mL) and stirred in an ice/water bath for 30 min. The ice bath is removed and the reaction mixture is allowed to stir overnight. The reaction mixture is then treated with 1 M HCl (150 mL). The organics are isolated and extracted once with 1 M HCl (100 mL) and once with saturated aqueous NaCl (20 mL). The organics are dried over MgSO₄, filtered, concentrated and purified on a column of ˜200 g of silica by eluting with 5% ethyl acetate in hexane (700 mL) followed by 10% ethyl acetate in hexane (700 mL) to afford 1-Methylcyclopropyl 4-nitrophenyl carbonate 21 (5.0 g) as an oil which solidifies after prolonged standing: ¹H NMR (CDCl₃) δ=8.28 (m, 2H), 7.38 (m, 2H), 1.66 (s, 3H), 1.07 (m, 2H), 0.76 (m, 2H); MS calcd. for C₁₁H₁₂NO₅ [M+H]⁺: 238.1. found: 237.8.

### Example D2

### 1-Methylcyclopropyl 4-(3-(6-(methylsulfonyl)-5,6,7,8-tetrahydropyrido[4,3-d]pyrimidin-2-yloxy)propyl)piperidine-1-carboxylate

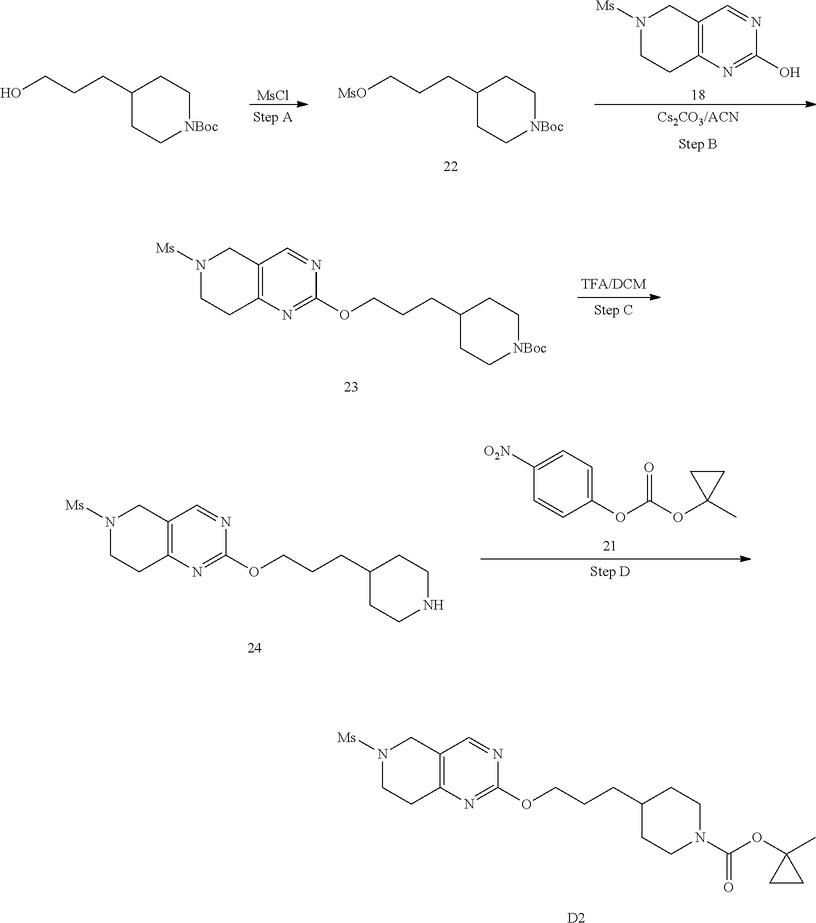

Step A: To a stirred solution of tert-butyl 4-(3-hydroxypropyl)piperidine-1-carboxylate (40.6 g, 167 mmol) and pyridine (27 mL, 184 mmol) in DCM (150 mL) is slowly added MsCl (14.3 mL, 184 mmol) over 30 min at 0° C. The reaction is then stirred at 0° C. for 1 h and then at rt overnight. The reaction mixture is partitioned between water (50 mL) and EtOAc (100 mL). The aqueous layer is separated and is further extracted with EtOAc (2×100 mL). The organics are combined and washed with brine (25 mL), dried (MgSO₄), and evaporated to give an amber-colored oil. The crude is purified by flash chromatography (SiO₂, EtOAc/hexanes gradient) to give tert-butyl 4-(3-(methylsulfonyloxy)propyl)piperidine-1-carboxylate 22 as a light yellow oil: ¹H NMR (400 MHz, CD₃CN) δ 4.18 (t, J=4.8 Hz, 2H), 4.00 (m, 2H), 2.99 (s, 3H), 2.67 (m, 2H), 1.72 (m, 2H), 1.65 (m, 2H), 1.43 (m, 1H), 1.41 (s, 9H), 1.30 (m, 2H), 1.01 (ddd, J=3.3, 9.6, 18.6 Hz, 2H); MS calcd. for C₉H₂₀NO₃S [M−Boc+H]⁺: 222.1. found: 221.9.

Step B: A mixture of 6-(methylsulfonyl)-5,6,7,8-tetrahydropyrido[4,3-d]pyrimidin-2-ol 18 (100 mg, 0.44 mmol), tert-butyl 4-(3-(methylsulfonyloxy)propyl)piperidine-1-carboxylate 22 (140 mg, 0.44 mmol) and cesium carbonate (180 mg, 0.55 mmol) in anhydrous dioxane (3 mL) is stirred in a sealed vial at 80° C. overnight. The reaction mixture is quenched with water (10 mL) and extracted with EtOAc (3×25 mL). The organic layer is washed with brine (5 mL), dried over MgSO₄, and evaporated to give a light yellow residue (180 mg). The crude is purified by flash chromatography (SiO₂, EtOAc/hexanes 50-100%) to give tert-butyl 4-(3-(6-(methylsulfonyl)-5,6,7,8-tetrahydropyrido[4,3-d]pyrimidin-2-yloxy)propyl)piperidine-1-carboxylate 23 as a light yellow solid: MS calcd. for C₂₁H₃₄N₄O₅S [M+H]⁺: 455.2. found: 455.2.

Step C: To a solution of tert-butyl 4-(3-(6-(methylsulfonyl)-5,6,7,8-tetrahydropyrido[4,3-d]pyrimidin-2-yloxy)propyl)piperidine-1-carboxylate 23 (103 mg, 0.227 mmol) in DCM (10 mL) at 0° C. is added TFA (2 mL). After stirring at rt for 5 h, the solvent is evaporated. The crude 6-(methylsulfonyl)-2-(3-(piperidin-4-yl)propoxy)-5,6,7,8-tetrahydropyrido[4,3-d]pyrimidine 24 is concentrated with MeOH repeatedly and then dried under high vacuum overnight. MS calcd. for C₁₆H₂₇N₄O₃S [M+H]⁺: 355.2. found: 355.1.

Step D: Intermediate 24 (approx 0.227 mmol) from above is dissolved in DCM (10 mL). TEA (0.1 mL, 0.72 mmol) is added at 0° C. followed by addition of carbonate 21 (60 mg, 0.25 mmol) as a solution in DCM (1 mL). After stirring at rt for 4 h, the reaction is diluted with EtOAc (25 mL), washed with 1N NaOH (5 mL), dried over MgSO₄, and concentrated to give a light yellow residue. The crude is purified by reverse-phase HPLC (H₂O/MeCN gradient) to give 1-methylcyclopropyl 44346-(methylsulfonyl)-5,6,7,8-tetrahydropyrido[4,3-d]pyrimidin-2-yloxy)propyl)piperidine-1-carboxylate D2 as a white powder: ¹H NMR (400 MHz, acetone-d₆) δ=8.40 (s, 1H), 4.43 (s, 2H), 4.30 (t, J=6.8 Hz, 2H), 4.05 (m, 2H), 3.64 (t, J=6.0 Hz, 2H), 2.96 (m, 2H), 2.95 (s, 3H), 2.70 (m, 2H), 1.82 (m, 2H), 1.71 (m, 2H), 1.50 (m, 1H), 1.49 (s, 3H), 1.41 (m, 2H), 1.04 (ddd, J=4.4, 12.8, 16.8 Hz, 2H), 0.78 (m, 2H), 0.58 (m, 2H); MS calcd. for C₂₁H₃₃N₄O₅S [M+H]⁺: 453.2. found: 453.2.

### Example D3

### 2-(3-(1-(5-Ethylpyrimidin-2-yl)piperidin-4-yl)propoxy)-6-(methylsulfonyl)-5,6,7,8-tetrahydropyrido[4,3-d]pyrimidine

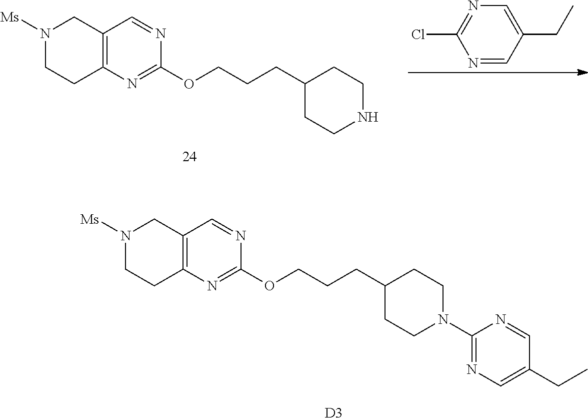

A mixture of intermediate 24, Cs₂CO₃ (110 mg, 0.34 mol) and 2-chloro-5-ethylpyrimidine (40 mg, 0.28 mmol) in dioxane:NMP (1:0.1 mL) is subjected to microwave irradiation (160° C., 20 min). The crude product is purified by reverse-phase HPLC (H₂O/MeCN gradient) to give 2-(3-(1-(5-ethylpyrimidin-2-yl)piperidin-4-yl)propoxy)-6-(methylsulfonyl)-5,6,7,8-tetrahydropyrido[4,3-d]pyrimidine D3 as an off-white solid: ¹H NMR (400 MHz, acetone-d₆) δ=8.41 (s, 1H), 8.19 (s, 2H), 4.75 (dt, J=13.2, 2.0 Hz, 2H), 4.43 (s, 2H), 4.32 (t, J=6.8 Hz, 2H), 3.64 (t, J=6.0 Hz, 2H), 2.97 (m, 2H), 2.95 (s, 3H), 2.83 (m, 2H), 2.45 (q, J=7.6 Hz, 2H), 1.86-1.77 (m, 4H), 1.24 (m, 1H), 1.42 (m, 2H), 1.16 (t, J=7.6 Hz, 3H), 1.11 (m, 2H); MS calcd. for C₂₂H₃₃N₆O₃S [M+H]⁺: 461.2. found: 461.2.

### Example D4

### N-(3-(1-(3-isopropyl-1,2,4-oxadiazol-5-yl)piperidin-4-yl)propyl)-6-(methylsulfonyl)-5,6,7,8-tetrahydropyrido[4,3-d]pyrimidin-2-amine

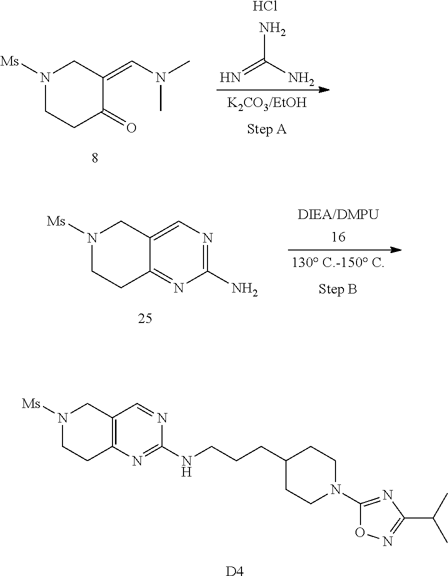

Step A: A suspension of 3-((dimethylamino)methylene)-1-(methylsulfonyl)piperidin-4-one 8 (3.28 g, 14.1 mmol), guanidine hydrochloride (5.40 g, 56.4 mmol) and potassium acetate (11.1 g, 112.8 mmol) in 95% EtOH (80 mL) is stirred at 80° C. for 2 days. The solvent is removed in vacuo. The residue is taken up in water and extracted with EtOAc (3×50 mL). The organic layer is washed with brine (10 mL), dried over MgSO₄, and evaporated to give a brownish residue. The crude product is purified by flash chromatography (SiO₂, MeOH/CH₂Cl₂ 0-10%) to give 6-(methylsulfonyl)-5,6,7,8-tetrahydropyrido[4,3-d]pyrimidin-2-amine 25 as a light yellow solid: ¹H NMR (400 MHz, CD₃CN) δ=8.08 (s, 1H), 5.35 (br s, 2H), 4.26 (s, 2H), 3.54 (t, J=4.5 Hz, 2H), 2.88 (s, 3H), 2.80 (t, J=4.5 Hz, 2H); MS calcd. for C₈H₁₃N₄O₂S [M+H]⁺: 229.1. found: 229.0.

Step B: A mixture of 6-(methylsulfonyl)-5,6,7,8-tetrahydropyrido[4,3-d]pyrimidin-2-amine 25 (40 mg, 0.175 mmol), mesylate 16 (64 mg, 0.192 mmol) and DIEA (60 uL, 0.35 mmol) in DMPU (1 mL) is stirred at 130° C. for 1 day, then 150° C. for 6 h. The crude is purified by reverse-phase HPLC (H₂O/MeCN gradient) to give N-(3-(1-(3-isopropyl-1,2,4-oxadiazol-5-yl)piperidin-4-yl)propyl)-6-(methylsulfonyl)-5,6,7,8-tetrahydropyrido[4,3-d]pyrimidin-2-amine D4 as a white powder: ¹H NMR (400 MHz, CD₃CN) δ=9.0 (br s, 1H), 8.2 (br s, 1H), 4.29 (s, 2H), 4.71 (m, 2H), 3.56 (t, J=4.5 Hz, 2H), 3.45 (J=5.4 Hz, 2H), 3.04 (dt, J=2.1, 9.9 Hz, 2H), 2.94 (t, J=4.5 Hz, 2H), 2.88 (s, 3H), 2.83 (m, 1H), 1.78-1.74 (m, 2H), 1.68-1.61 (m, 2H), 1.52 (m, 1H), 1.34 (m, 2H), 1.23-1.16 (m, 2H), 1.21 (d, J=5.1 Hz, 6H); MS calcd. for C₂₁H₃₄N₇O₃S [M+H]⁺: 464.2. found: 464.2.

### Example D5

### N-(3-(1-(5-Ethylpyrimidin-2-yl)piperidin-4-yl)propyl)-6-(methylsulfonyl)-5,6,7,8-tetrahydropyrido[4,3-d]pyrimidin-2-amine

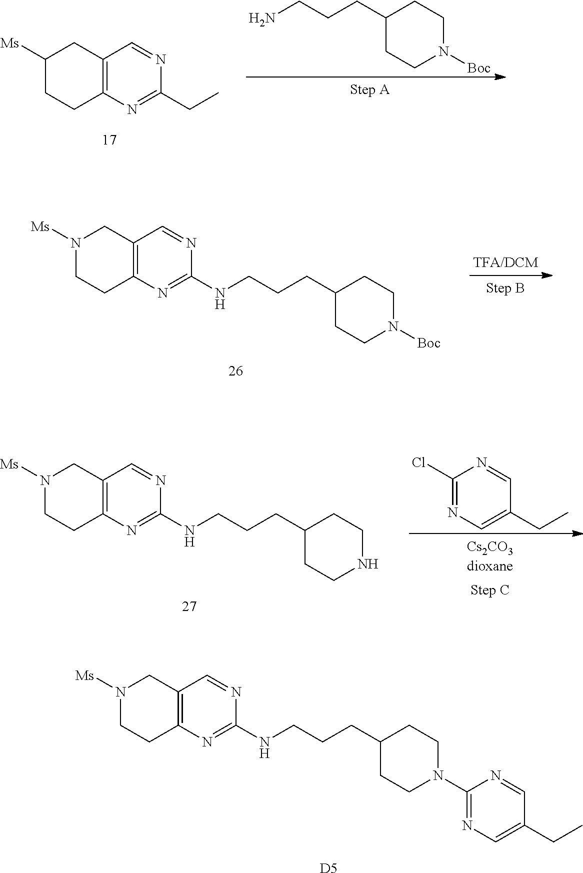

Step A: 2-Methoxy-6-(methylsulfonyl)-5,6,7,8-tetrahydropyrido[4,3-d]pyrimidine 17 (0.30 g, 1.17 mmol) in neat tert-butyl 4-(3-aminopropyl)piperidine-1-carboxylate (0.82 g, 3.38 mmol) is stirred at 150° C. oil bath for 24 h. The reaction is purified by flash chromatography (SiO₂, EtOAc/hexanes 30-80%) to give tert-butyl 4-(3-(6-(methylsulfonyl)-5,6,7,8-tetrahydropyrido[4,3-d]pyrimidin-2-ylamino)propyl)piperidine-1-carboxylate 26 as a light yellow solid: MS calcd. for C₂₁H₃₆N₅O₄S [M+H]⁺: 454.3. found: 454.2

Step B: To a solution of 26 (270 mg, 0.59 mmol) in DCM (10 mL) is added TFA (1 mL) at 0° C. After stirring at rt for 1 h, the solvents are evaporated. The residue is repeatedly concentrated from MeOH to remove excess TFA. The crude 6-(methylsulfonyl)-N-(3-(piperidin-4-yl)propyl)-5,6,7,8-tetrahydropyrido[4,3-d]pyrimidin-2-amine 27 is used without further purification: MS calcd. for C₁₆H₂₈N₅O₂S [M+H]⁺: 354.2. found: 354.2.

Step C: A suspension of 6-(methylsulfonyl)-N-(3-(piperidin-4-yl)propyl)-5,6,7,8-tetrahydropyrido[4,3-d]pyrimidin-2-amine 27 (42 mg, 0.12 mmol), 2-chloro-5-ethylpyrimidine (30 mg, 0.21 mmol) and cesium carbonate (137 mg, 0.42 mmol) in anhydrous dioxane (1 mL) is stirred in a sealed vial at 100° C. for 14 h. The reaction is purified by reverse-phase HPLC (H₂O/MeCN gradient) to give N-(3-(1-(5-ethylpyrimidin-2-yl)piperidin-4-yl)propyl)-6-(methylsulfonyl)-5,6,7,8-tetrahydropyrido[4,3-d]pyrimidin-2-amine D5 as a white powder (30 mg, TFA salt): ¹H NMR (400 MHz, acetone-d₆) δ=8.18 (s, 2H), 8.11 (s, 1H), 6.19 (t, J=5.2 Hz, 1H), 4.74 (dt, J=13.8, 2.4 Hz, 2H), 4.28 (s, 2H), 3.57 (t, J=6.0 Hz, 2H), 3.39 (q, J=7.2 Hz, 2H), 2.92 (s, 3H), 2.83-2.76 (m, 4H), 2.45 (q, J=7.6 Hz, 2H), 1.76 (m, 2H), 1.68 (m, 2H), 1.58 (m, 1H), 1.34 (m, 2H), 1.16 (t, J=7.6 Hz, 3H), 1.11 (m, 2H); MS calcd. for C₂₂H₃₄N₇O₂S [M+H]⁺: 460.2. found: 460.3.

### Example D6

### N-(3-(1-(5-Ethylpyrimidin-2-yl)piperidin-4-yl)propyl)-N-methyl-6-(methylsulfonyl)-5,6,7,8-tetrahydropyrido[4,3-d]pyrimidin-2-amine

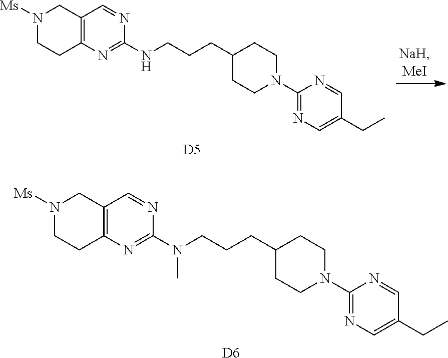

Sodium hydride (20 mg, 0.5 mmol, 60% in mineral oil) is added to a solution of N-(3-(1-(5-ethylpyrimidin-2-yl)piperidin-4-yl)propyl)-6-(methylsulfonyl)-5,6,7,8-tetrahydropyrido[4,3-d]pyrimidin-2-amine D5 (15 mg, 0.033 mmol) in DMF (1.5 mL). After stirring at rt for 20 min, iodomethane (41 μL, 0.66 mmol) is added. The reaction is stirred at rt for 1 h. Purification by reverse-phase HPLC (H₂O/MeCN gradient) yielded N-(3-(1-(5-ethylpyrimidin-2-yl)piperidin-4-yl)propyl)-N-methyl-6-(methylsulfonyl)-5,6,7,8-tetrahydropyrido[4,3-d]pyrimidin-2-amine D6 as a white powder: MS calcd. for C₂₃H₃₆N₇O₂S [M+H]⁺: 474.3. found: 474.3.

By repeating the procedure described in the above example D1-D6, using appropriate starting materials, the following compounds of Formula I, as identified in Table 3, are obtained.

TABLE 3

| Ex. | | |
|---|---|---|
| # | Structure | NMR and/or ESMS |
| D7 | 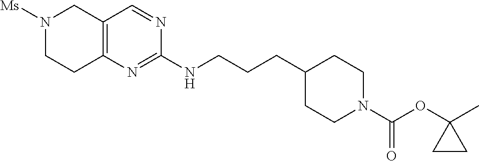 | ¹H NMR (400 MHz, acetone-d₆) δ = 8.11 (s, 1H), 6.23 (br s, 1H), 4.28 (s, 2H), 4.05 (m, 2H), 3.57 (t, J = 6.0 Hz, 2H), 3.37 (q, J = 6.8 Hz, 2H), 2.92 (s, 3H), 2.95 (s, 3H), 2.80 (t, J = 6.0 Hz, 2H), 2.60 (m, 2H), 1.70-1.60 (m, 4H), 1.49 (s, 3H), 1.48 (m, 1H), 1.32 (m, 2H), 1.01 (ddd, J = 4.4, 12.8, 16.8 Hz, 2H), 0.77 (m, 2H), 0.57 (m, 2H); MS calcd. for C₂₁H₃₄N₅O₄S [M + H]⁺: 452.2, found: 452.3. |
| D8 | 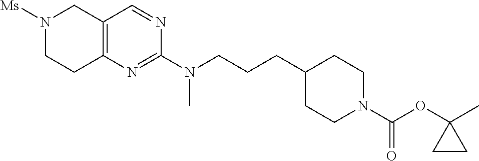 | ¹H NMR (400 MHz, CD₃CN) δ 8.16 (s, 1H), 4.28 (s, 2), 3.95 (br s, 2H), 3.61 (t, J = 7.6 Hz, 2H), 3.55 (t, J = 6.0 Hz, 2H), 3.13 (s, 3H), 2.89 (t, J = 6.2 Hz, 2H), 2.87 (s, 3H), 2.68 (m, 2H), 1.62 (m, 4H), 1.45 (s, 3H), 1.44 (m, 1H), 1.22 (dd, J = 6.8, 15.6 Hz, 2H), 0.98 (ddd, J = 4.0, 12.4, 24.4 Hz, 2H), 0.78 (dd, J = 5.6, 7.2 Hz, 2H), 0.58 (m, 2H); MS calcd. for C₂₂H₃₆N₅O₄S [M + H]⁺: 466.3, found: 466.2 |
| D9 | 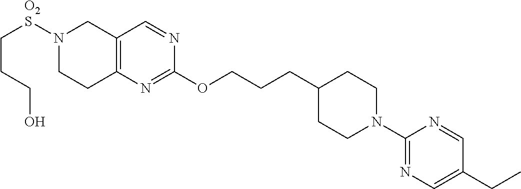 | ¹H NMR (400 MHz, CD₃Cl) δ 8.24 (s, 1H), 8.15 (s, 2H), 4.68 (m, 2H), 4.45 (s, 2H), 4.33 (t, J = 6.4 Hz, 2H), 4.18 (t, J = 6.0 Hz, 2H), 3.67 (t, J = 6.0 Hz, 2H), 3.49 (s, 1H), 3.08 (m, 2H), 3.00 (t, J = 6.0 Hz, 2H), 2.83 (dt, J = 2.4, 13.2 Hz, 2H), 2.44 (quartet, J = 7.6 Hz, 2H), 2.17 (m, 2H), 1.89-1.77 (m, 4H), 1.55 (m, 1H), 1.45 (m, 2H), 1.18 (t, J = 7.6 Hz, 3H), 1.19 (m, 2H); MS calcd. for C₂₄H₃₇N₆O₄S [M + H]⁺: 505.3, found: 505.3. |

### Intermediate 31: 2-(1-(5-Ethylpyrimidin-2-yl)piperidin-4-yloxy)ethyl methanesulfonate

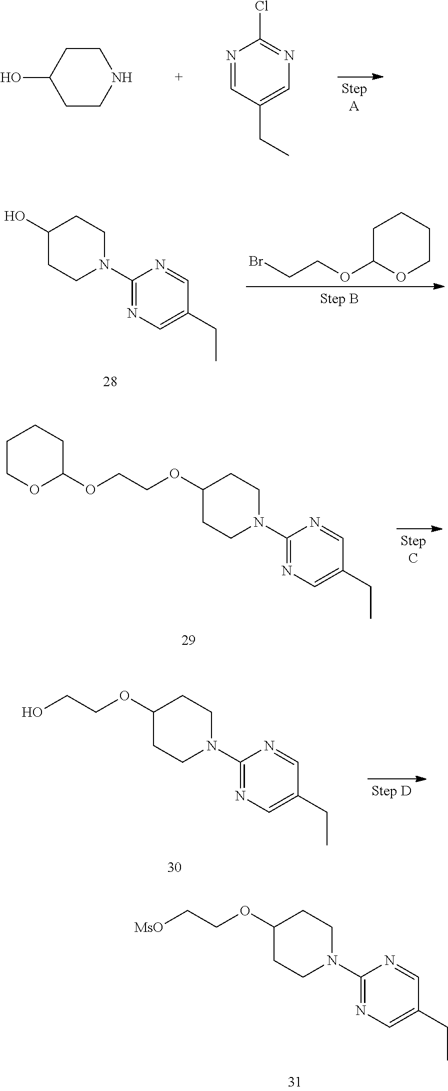

Step A: 4-Hydroxypiperidine (1 g, 9.9 mmol), 2-chloro-5-ethylpyrimide (940 mg, 6.6 mmol), and cesium carbonate (4.3 g, 13.2 mmol) are dissolved in dioxane (15 mL) and the mixture is subjected to microwave irradiation (160° C., 20 min). The mixture is cooled, filtered, diluted with H₂O and extracted with EtOAc (40 mL). The organic layer is washed with brine (20 mL), dried (MgSO₄) and concentrated. The residue is purified by flash column chromatography (SiO₂, EtOAc/Hexane gradient) to afford the hydroxyl intermediate 28 as a colorless oil which solidified under high vacuum: MS calcd. for C₁₁H₁₈N₃O [M+H]⁺: 208.1. found: 208.1.

Step B: To intermediate 28 (500 mg, 2.4 mmol) in DMF (10 mL) is added sodium hydride (60% in mineral oil, 144 mg, 3.6 mmol) at 0° C. The mixture is stirred for 30 min at rt, then 2-(2-bromoethoxy)tetrahydro-2H-pyran (729 uL, 4.8 mmol) is added and the mixture is heated at 80° C. for 1 h. The mixture is diluted with H₂O (20 mL) and extracted with EtOAc (20 mL). The organic layer is washed with sat aq NH₄Cl (20 mL) and H₂O (3×20 mL), then is dried (MgSO₄), filtered, concentrated, and purified by flash column chromatography (SiO₂, EtOAc/hexane gradient) to afford 5-ethyl-2-(4-(2-(tetrahydro-2H-pyran-2-yloxy)ethoxy)piperidin-1-yl)pyrimidine 29: MS calcd. for C₁₈H₃₀N₃O₃ [M+H]⁺: 336.2. found: 336.2.

Step C: 5-Ethyl-2-(4-(2-(tetrahydro-2H-pyran-2-yloxy)ethoxy)piperidin-1-yl)pyrimidine 29 (360 mg, 1.1 mmol) is dissolved in MeOH (5 mL) and para-toluenesulfonic acid hydrate (209 mg, 1.1 mmol) is added and stirred at rt for 1 h. The mixture is diluted with H₂O (10 mL) and extracted with EtOAc (20 mL). The organic layer is washed with sat aq NaHCO₃, then dried (Na₂SO₄), filtered and concentrated to provide 2-(1-(5-ethylpyrimidin-2-yl)piperidin-4-yloxy)ethanol 30, which is used without purification in Step D: MS calcd. for C₁₃H₂₂N₃O₂ [M+H]⁺: 252.2. found: 252.1.

Step D: 2-(1-(5-Ethylpyrimidin-2-yl)piperidin-4-yloxy)ethanol 30 (276 mg, 1.1 mmol) and NEt₃ (307 uL, 2.2 mmol) are dissolved in DCM (5 mL) and cooled to 0° C. Methanesulfonyl chloride (127 uL, 1.7 mmol) is added and the reaction mixture is stirred for 10 min. The mixture is concentrated and the residue is purified by flash column chromatography (SiO₂, EtOAc/Hexane gradient) to afford the title compound 31 as a colorless oil: ¹H NMR (400 MHz, CDCl₃) δ=8.17 (s, 2H), 4.39-4.37 (m, 2H), 4.31-4.25 (m, 2H), 3.79-3.76 (m, 2H), 3.61 (septet, 1H, J=3.6 Hz), 3.38-3.31 (m, 2H), 3.06 (s, 3H), 2.46 (t, 2H, J=7.6, 15.2 Hz), 1.96-1.91 (m, 2H), 1.62-1.54 (m, 2H), 1.18 (t, 3H, J=7.6 Hz); MS calcd. for [M+H]⁺ C₁₄H₂₃N₃O₄S: 330.1. found: 330.1.

### Example E1

### 2-(2-(1-(5-ethylpyrimidin-2-yl)piperidin-4-yloxy)ethoxy)-6-(methylsulfonyl)-5,6,7,8-tetrahydropyrido[4,3-d]pyrimidine

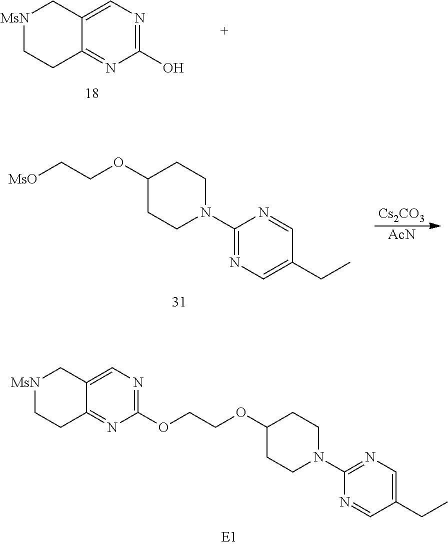

6-(Methylsulfonyl)-5,6,7,8-tetrahydropyrido[4,3-d]pyrimidin-2-ol 18 (50 mg, 0.22 mmol) and 2-(1-(5-ethylpyrimidin-2-yl)piperidin-4-yloxy)ethyl methanesulfonate 33 (72 mg, 0.22 mmol) are dissolved in acetonitrile (5 mL). Cs₂CO₃ (142 mg, 0.44 mmol) is added and the mixture is heated at 80° C. for 12 h. The mixture is cooled, filtered, concentrated, and purified by reverse-phase HPLC (H₂O/MeCN gradient) to give 2-(2-(1-(5-ethylpyrimidin-2-yl)piperidin-4-yloxy)ethoxy)-6-(methylsulfonyl)-5,6,7,8-tetrahydropyrido[4,3-d]pyrimidine E1 as a white powder: ¹H NMR (400 MHz, CDCl₃) δ=8.38 (s, 2H), 8.22 (s, 1H), 4.46 (m, 2H), 4.35 (s, 2H), 3.89 (m, 2H), 3.82 (m, 4H), 3.70 (m, 1H), 3.57 (m, 2H), 2.98 (m, 2H), 2.84 (s, 3H), 2.50 (q, J=7.6 Hz, 2H), 1.79 (m, 4H), 1.18 (t, J=7.6 Hz, 3H); MS calcd. for C₂₁H₃₁N₆O₄S [M+H]⁺: 463.2. found: 463.2.

### Intermediate 34: 3-(1-(5-Ethylpyrimidin-2-yl)piperidin-4-yl)propyl methanesulfonate

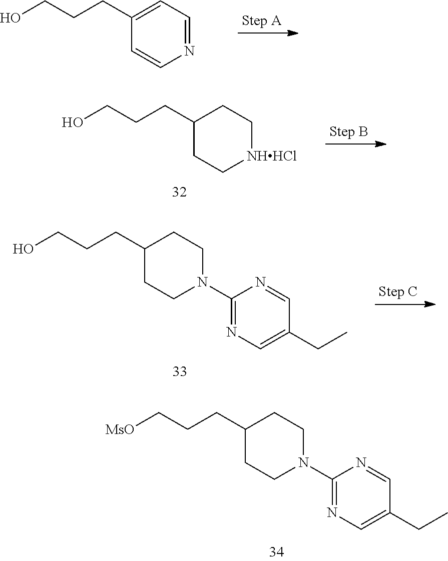

Step A: To a 500 mL hydrogenation flask is added a solution of 3-(pyridin-4-yl)propan-1-ol (25 g, 182.5 mmol) in ethanol (200 mL). Concentrated HCl (25 mL) is added followed by addition of PtO₂ (200 mg). The mixture is subjected to H₂ (60 psi) in a Parr shaker for 20 h. Then the solvent is removed under reduced pressure and the residue is dried under high vacuum overnight to afford 3-(piperidin-4-yl)propan-1-ol hydrochloride 32 (31.6 g). MS calcd. for [M+H]⁺ C₈H₁₈NO: 144.1. found: 144.1.

Step B: A round bottom flask is charged with 3-(piperidin-4-yl)propan-1-ol hydrochloride 32 (1.8 g, 10 mmol), 2-chloro-5-ethylpyrimidine (1.44 g, 10.1 mmol), Cs₂CO₃ (7 g, 10.1 mmol) in DMF (25 mL). The mixture is heated to 120° C. for 20 h. Then it is cooled to rt and EtOAc (100 mL) is added. The mixture is separated, and the organic layer is washed with water (3×30 mL) and brine (30 mL), then dried over Na₂SO₄. The solvents are removed in vacuo and the remainder is purified via flash column chromatography (EtOAc:hexane=2:1) to give 3-(1-(5-ethylpyrimidin-2-yl)piperidin-4-yl)propan-1-ol 33 (1.78 g) as a solid. MS calcd. for [M+H]⁺ C₁₄H₂₄N₃O: 250.1. found: 250.1.

Step C: To a solution of 3-(1-(5-ethylpyrimidin-2-yl)piperidin-4-yl)propan-1-ol 33 (1.25 g, 5 mmol) in CH₂Cl₂ (20 mL) is added Et₃N (1 mL, 7.2 mmol). The mixture is cooled to 0° C., then MsCl (0.41 mL, 5.28 mmol) is added slowly. After the addition is complete, the reaction mixture is stirred for 3 h at rt, then quenched with water. CH₂Cl₂ (20 mL) is added and the mixture is washed with water (20 mL) and brine (2×20 mL). The organics are concentrated and filtered through a short silica gel plug (10 g, washed with EtOAc:Hexane=1:2) to afford the desired intermediate 3-(1-(5-ethylpyrimidin-2-yl)piperidin-4-yl)propyl methanesulfonate 34 (1.45 g): MS calcd. for [M+H]⁺C₁₅H₂₆N₃O₃S: 328.1. found: 328.1.

### Example F1

### 2-(3-(1-(5-Ethylpyrimidin-2-yl)piperidin-4-yl)propoxy)-6-(methylsulfonyl)-5,6,7,8-tetrahydro-1,6-naphthyridine

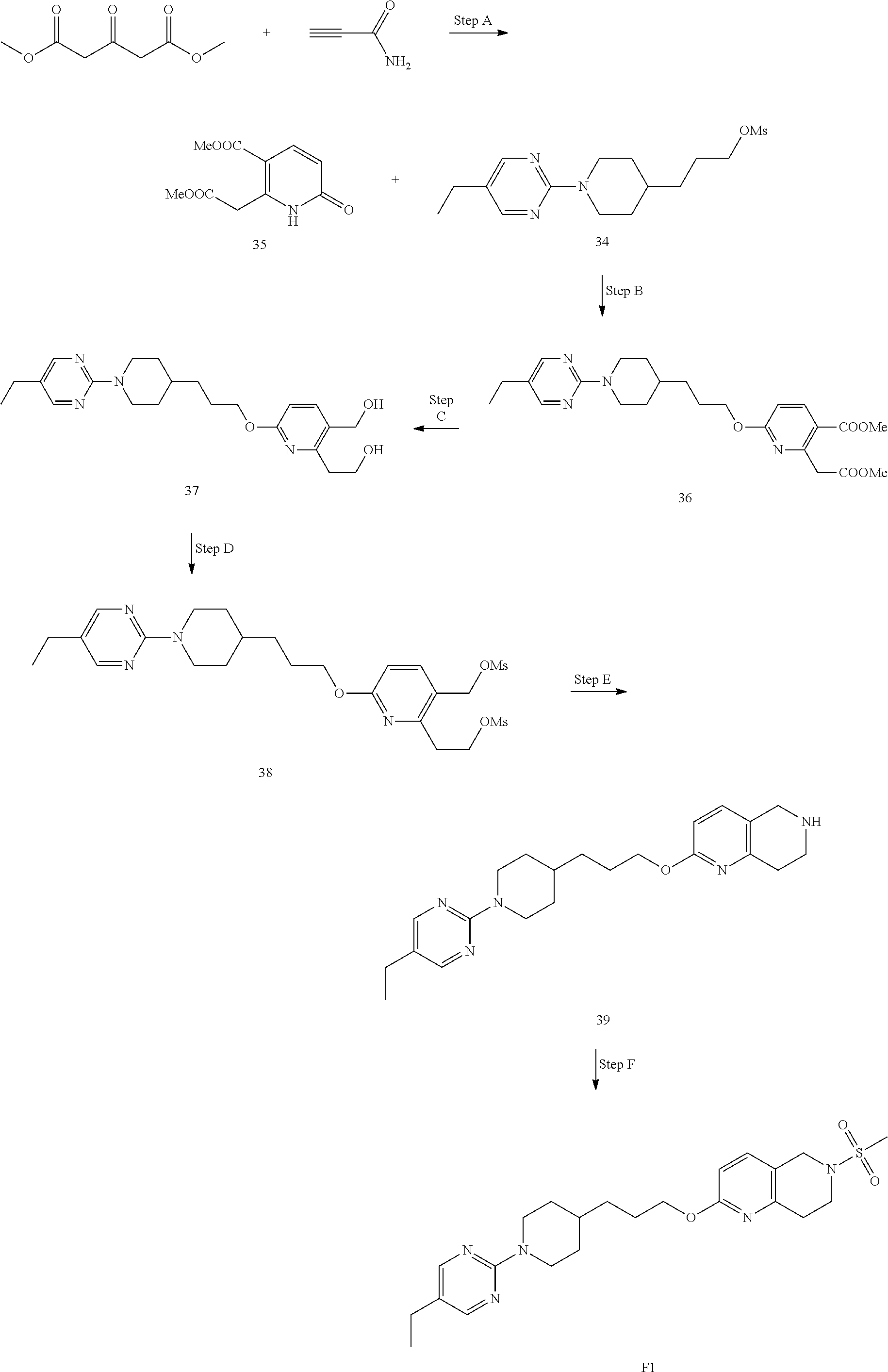

Step A: To a solution of dimethyl 3-oxopentanedioate (4.8 g, 28.8 mmol) in water (5 mL) is added saturated aqueous sodium carbonate until the pH is adjusted to 8-9. Then the mixture is cooled to 0° C. with an ice-bath. After addition of a solution of propiolamide (1.5 g, 21.7 mmol) in water (2 mL) the resulting mixture is stirred at 0° C. for 20 h. It is then extracted with CHCl₃ (3×50 mL). The extracts are combined, washed with brine and dried over Na₂SO₄. Concentration and re-crystallization of the crude product from MeOH gave methyl 2-(2-methoxy-2-oxoethyl)-6-oxo-1,6-dihydropyridine-3-carboxylate 35. MS calculated for [M+H]⁺ C₁₀H₁₂NO₅, 226.1. found: 226.1.

Step B: To a round bottom flask is added methyl 2-(2-methoxy-2-oxoethyl)-6-oxo-1,6-dihydropyridine-3-carboxylate 35 (0.6 g, 2.69 mmol), 3-(1-(5-ethylpyrimidin-2-yl)piperidin-4-yl)propyl methanesulfonate 34 (0.86 g, 2.64 mmol), Cs₂CO₃ (1.2 g, 3.69 mmol) and CHCl₃ (20 mL). The mixture is stirred at rt for 1 day and then heated to 60° C. for an additional day. It is then filtered, and the solid is washed with CHCl₃ (30 mL). The organics is combined and the solvent is removed under reduced pressure. The crude is purified by flash chromatography (SiO₂, EtOAc/Hexanes 1:1) to afford methyl 6-(3-(1-(5-ethylpyrimidin-2-yl)piperidin-4-yl)propoxy)-2-(2-methoxy-2-oxoethyl)nicotinate 36. MS calculated for [M+H]⁺ C₂₄H₃₃N₄O₅, 457.2. found: [M+H]⁺: 457.2.

Step C: To a solution of methyl 6-(3-(1-(5-ethylpyrimidin-2-yl)piperidin-4-yl)propoxy)-2-(2-methoxy-2-oxoethyl)nicotinate 36 (0.12 g, 0.26 mmol) in dry THF (15 mL) is added a solution of DIBAL-H (2 mL, 1 M in THF) at −78° C. The resulting mixture is stirred for 5 h while the temperature is kept between −78° C. and −50° C., then quenched with saturated NH₄Cl solution. The mixture is warmed to rt and EtOAc (20 mL) is added. The organic layer is washed with brine and dried over Na₂SO₄. The solvents are removed under reduced pressure and the crude 2-(6-(3-(1-(5-ethylpyrimidin-2-yl)piperidin-4-yl)propoxy)-3-(hydroxymethyl)pyridin-2-yl)ethanol 37 is used directly in the next step without purification.

Step D: A solution of crude 2-(6-(3-(1-(5-ethylpyrimidin-2-yl)piperidin-4-yl)propoxy)-3-(hydroxymethyl)pyridin-2-yl)ethanol 37 (0.1 g, 0.25 mmol) in DCM (10 mL) is cooled to 0° C. Et₃N (200 uL, 1.4 mmol) is added. While the mixture is stirred at 0° C., MsCl (60 uL, 0.86 mmol) is added slowly. The mixture is stirred at 0° C. for 3 h, warmed to rt and stirred for an additional 2 h. It is then again cooled down to 0° C. and quenched with water. The organic layer is separated, washed with brine and dried over Na₂SO₄. The solvents is removed under reduced pressure and the crude is purified by flash chromatography (SiO₂, EtOAc/Hexanes 1:1) to provide (6-(3-(1-(5-ethylpyrimidin-2-yl)piperidin-4-yl)propoxy)-2-(2-(methylsulfonyloxy)ethyl)pyridin-3-yl)methyl methanesulfonate 38. MS calculated for [M+H]⁺ C₂₄H₃₇N₄O₄S₂: 557.2. Found: 557.2.

Step E: A solution of (6-(3-(1-(5-ethylpyrimidin-2-yl)piperidin-4-yl)propoxy)-2-(2-(methylsulfonyloxy)ethyl)pyridin-3-yl)methyl methanesulfonate 38 (0.09 g, 0.17 mmol) in 1 M NH₃ in isopropanol (10 mL) is subjected to microwave irradiation (160° C., 30 min). The mixture is then cooled down to rt, the solvents is removed under reduced pressure. Purification of the crude by flash chromatography (SiO₂, CHCl₃/MeOH 20:1) afforded 2-(3-(1-(5-ethylpyrimidin-2-yl)piperidin-4-yl)propoxy)-5,6,7,8-tetrahydro-1,6-naphthyridine 39. MS calculated for [M+H]⁺ C₂₂H₃₁N₅O: 382.3. Found: 382.3.

Step F: To a dry flask is added 2-(3-(1-(5-Ethylpyrimidin-2-yl)piperidin-4-yl)propoxy)-5,6,7,8-tetrahydro-1,6-naphthyridine 39 (10 mg, 0.028 mmol) and DCM (3 mL). The solution is cooled to 0° C. with an ice-bath. Et₃N (0.1 mL, 0.07 mmol) is added and the solution is stirred at 0° C. for 10 min. MsCl (0.01 mL, 0.09 mmol) is added. The mixture is stirred at 0° C. for 2 h, then quenched with water (0.5 mL). The organics are separated and washed with brine (2 mL). It is dried over Na₂SO₄ and the solvents are removed under reduced pressure. Purification of the crude by flash chromatography (SiO₂, EtOAc/Hexanes 1:1) provided 2-(3-(1-(5-ethylpyrimidin-2-yl)piperidin-4-yl)propoxy)-6-(methylsulfonyl)-5,6,7,8-tetrahydro-1,6-naphthyridine F1. ¹H NMR (400 MHz, CDCl₃) δ=8.16 (2H, s); 7.28 (1H, d, J=8.4 Hz); 7.45 (1H, d, J=8.4 Hz); 4.78 (1H, brs); 4.66 (1H, brs); 4.47 (2H, s); 3.98 (2H, t, J=6.0 Hz); 3.45 (2H, t, J=6.0 Hz); 2.89 (2H, t, J=6.2 Hz); 2.86 (2H, dt, J=2.2 Hz, J=13.0 Hz); 2.81 (3 H, s); 2.44 (2H, q, J=7.6 Hz); 1.80 (3H, m); 1.55 (2H, m); 1.42 (2H, m); 1.37 (2H, m); 1.36 (3H, t, J=7.6 Hz) ppm. MS calculated for [M+H]⁺ C₂₃H₃₄N₅O₃S: 460.2. Found: [MH+]: 460.2.

### Intermediate 43: 2-(4-((Azetidin-3-yloxy)methyl)piperidin-1-yl)-5-ethylpyrimidine hydrochloride

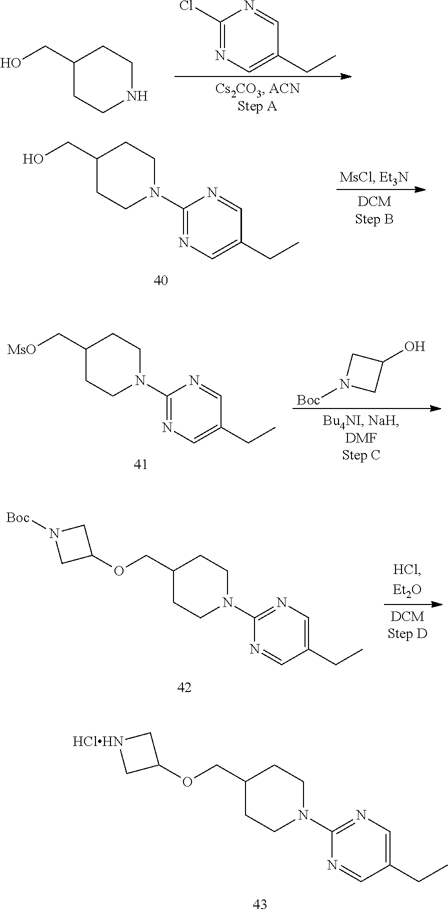

Step A: Piperidin-4-ylmethanol (11.85 g, 103 mmol) and 2-chloro-5-ethylpyrimidine (10.98 g, 77 mmol) are dissolved in dry acetonitrile (50 mL). Powdered cesium carbonate (41.44 g, 127 mmol) is added and the mixture is stirred vigorously at 75° C. for 18 h. Cooling to rt, filtration, washing the solids with more acetonitrile, and concentration of the filtrate yielded an oil. The residue is dissolved in ethyl acetate (120 mL), washed with water (100 mL), sat. aqueous NH₄Cl solution, and brine, dried over Na₂SO₄ and concentrated to yield (1-(5-ethylpyrimidin-2-yl)piperidin-4-yl)methanol 40 as a thick, near-colorless oil. ¹H NMR (CDCl₃, 400 MHz): δ 8.21 (s, 2H), 4.79 (d, J=13.2 Hz, 2H), 3.54 (d, J=5.16 Hz, 2H), 2.94 (t, J=12.6 Hz, 2H), 2.48 (q, J=7.6 Hz, 2H), 1.86 (d, J=13.4 Hz, 2H), 1.81 (m, 1H), 1.43 (br. s, 1H), 1.26 (m, 2H), 1.20 (t, J=7.6 Hz, 3H); MS (m/z) calculated for C₁₂H₂₀H₃O⁺ (M+H⁺): 222.16. found 222.1.

Step B: (1-(5-Ethylpyrimidin-2-yl)piperidin-4-yl)methanol 40 (6.0 g, 27.1 mmol) and triethylamine (10 mL, 72 mmol) are dissolved in dichloromethane (150 mL). Methanesulfonyl chloride (3 mL, 38.6 mmol) is slowly added, with stirring. The mixture is stirred at rt for 30 min, then washed with sat. NaHCO₃, dried over Na₂SO₄ and concentrated. The residue is purified by silicagel chromatography (10→70% EtOAc in hexane gradient) to yield (1-(5-ethylpyrimidin-2-yl)piperidin-4-yl)methyl methanesulfonate 41 as an oil that solidified to a white, glassy solid. ¹H NMR (CDCl₃, 400 MHz): δ 8.18 (s, 2H), 4.77 (d, J=13.4 Hz, 2H), 4.10 (d, J=6.6 Hz, 2H), 3.04 (s, 3H), 2.84 (td, J=13.2, 2.5 Hz, 2H), 2.46 (q, J=7.6 Hz, 2H), 2.07 (m, 1H), 1.86 (d, J=13.4 Hz, 2H), 1.27 (m, 2H), 1.19 (t, J=7.6 Hz, 3H); MS (m/z) calculated for C₁₃H₂₂N₃O₃S⁺ (M+H⁺): 300.14. found 300.1.

Step C: (1-(5-Ethylpyrimidin-2-yl)piperidin-4-yl)methyl methanesulfonate 41 (0.72 g, 2.4 mmol), tert-butyl 3-hydroxyazetidine-1-carboxylate (0.46 g, 2.66 mmol) and tetra-n-butylammonium iodide (0.35 g 0.95 mmol) are dissolved in dry dimethylformamide (6 mL). Sodium hydride (60% in mineral oil; 0.25 g, 6.2 mmol) is carefully added and the mixture is stirred in a preheated bath at 80° C. for 15 min. Cooling to rt, adding sat. NH₄Cl aqueous solution (2 mL) and extracting with dichloromethane (2×50 mL) are followed by washing with water (2×50 mL), drying over Na₂SO₄, and concentration. Purification by silicagel chromatography (0→75% EtOAc in hexane gradient) yielded tert-butyl 3-((1-(5-ethylpyrimidin-2-yl)piperidin-4-yl)methoxy)azetidine-1-carboxylate 42 as an oil. ¹H NMR (CDCl₃, 400 MHz): δ 8.17 (s, 2H), 4.73 (d, J=13.3 Hz, 2H), 4.18 (m, 1H), 4.06 (dd, J=9.3, 6.4 Hz, 2H), 3.80 (dd, J=10.0, 4.9 Hz, 2H), 3.21 (d, J=6.1 Hz, 2H), 2.87 (td, J=13.2, 2.5 Hz, 2H), 2.46 (q, J=7.6 Hz, 2H), 1.85 (m, 1H), 1.82 (d, J=12.4 Hz, 2H), 1.44 (s, 9H), 1.23 (m, 2H), 1.18 (t, J=7.6 Hz, 3H); MS (m/z) calculated for C₂₀H₃₃N₄O₃⁺ (M+H⁺): 377.25. found 377.2.

Step D: tert-Butyl 3-((1-(5-ethylpyrimidin-2-yl)piperidin-4-yl)methoxy) azetidine-1-carboxylate 42 (0.46 g, 1.2 mmol) is dissolved in dichloromethane (5 mL) and treated with hydrogen chloride (2M solution in diethyl ether; 2.0 mL, 4 mmol). The mixture is stirred at rt for 20 h. Concentration yielded 2-(4-((azetidin-3-yloxy)methyl)piperidin-1-yl)-5-ethylpyrimidine hydrochloride 43 as an oil. MS (m/z) calculated for C₁₅H₂₅N₄O⁺ (M+H⁺): 277.20. found 277.2.

### Intermediate 45: 2-(3-((1-(5-ethylpyrimidin-2-yl)piperidin-4-yl)methoxy)azetidin-1-yl)-6-(methylsulfonyl)-5,6,7,8-tetrahydropyrido[4,3-d]pyrimidine

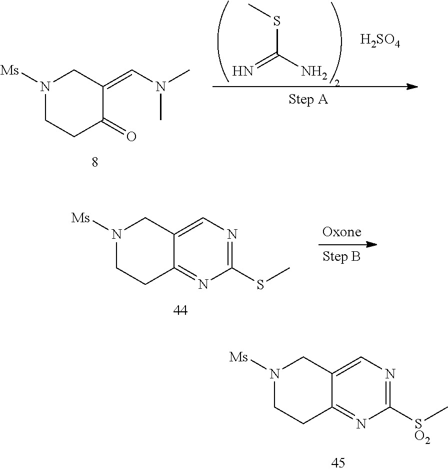

Step A: (Z)-3-((Dimethylamino)methylene)-1-(methylsulfonyl)piperidin-4-one 8 (1.09 g, 4.7 mmol) and bis-(methyl carbamimidothioate) sulfate (0.84 g, 6 mmol) are suspended in 1 mL water. Sodium hydroxide aqueous solution (1.0 M, 5 mL, 5 mmol) is added (initial pH=5) and the mixture is heated at 75° C. for 30 min. The mixture is cooled, diluted with water (50 mL) and filtered. The resulting 6-(methylsulfonyl)-2-(methylthio)-5,6,7,8-tetrahydropyrido[4,3-d]pyrimidine 44 is washed with water and air-dried. ES-LCMS calcd. for C₉H₁₄N₃O₂S₂ (M+H⁺) 260.2. found 260.1. ¹H NMR (dmso-d₆, 400 MHz): δ 8.50 (s, 1H), 4.38 (s, 2H), 3.53 (t, J=6.0 Hz, 2H), 2.99 (s, 3H), 2.93 (t, J=6.0 Hz, 2H), 2.50 (s, 3H).

Step B: Oxone® (7.64 g, 12.4 mmol) is suspended in water (15 mL), 6-(methylsulfonyl)-2-(methylthio)-5,6,7,8-tetrahydropyrido[4,3-d]pyrimidine 44 (1.01 g, 3.9 mmol) and acetonitrile (20 mL) are added and the mixture is vigorously stirred at 60° C. for 4.5 h. The mixture is cooled, the acetonitrile is removed in vacuo, water is added (120 mL) and the resulting solids are filtered, washed with water and air-dried to yield 2,6-bis(methylsulfonyl)-5,6,7,8-tetrahydropyrido[4,3-d]pyrimidine 45 as a white solid. ES-LCMS calcd. for C₉H₁₄N₃O₄S₂ (M+H⁺) 292.2. found 292.1. ¹H NMR (dmso-d₆, 400 MHz): δ 8.92 (s, 1H), 4.59 (s, 2H), 3.61 (t, J=6.0 Hz, 2H), 3.40 (s, 3H), 3.13 (t, J=6.0 Hz, 2H), 3.03 (s, 3H).

### Example G1

### 2-(3-((1-(5-Ethylpyrimidin-2-yl)piperidin-4-yl)methoxy)azetidin-1-yl)-6-(methylsulfonyl)-5,6,7,8-tetrahydropyrido[4,3-d]pyrimidine

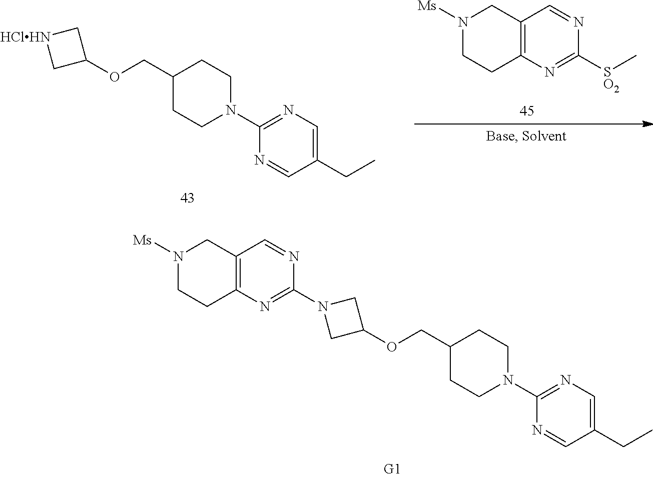

2,6-Bis(methylsulfonyl)-5,6,7,8-tetrahydropyrido[4,3-d]pyrimidine (0.45 g, 1.5 mmol) 45 is dissolved in NMP (3 mL) and heated to 80° C. until a solution is obtained. 2-(4-((Azetidin-3-yloxy)methyl)piperidin-1-yl)-5-ethylpyrimidine hydrochloride (1.5 mmol) 43 in NMP (2 mL) and ethyldiisopropyl amine (0.4 mL, 2.4 mmol) are added and the mixture is stirred at 80° C. for 20 h. Cooling to rt and extracting with dichloromethane (2×50 mL), followed by washing with water (2×50 mL), drying over Na₂SO₄, and concentration, then purification by silicagel chromatography (25→100% EtOAc in hexane gradient, followed by 0→45% ACN in EtOAc) yields 2-(3-((1-(5-ethylpyrimidin-2-yl)piperidin-4-yl)methoxy)azetidin-1-yl)-6-(methylsulfonyl)-5,6,7,8-tetrahydropyrido[4,3-d]pyrimidine G1 as a white solid. ¹H NMR (dmso-d₆, 400 MHz): δ 8.22 (s, 3H), 4.61 (d, J=13.2 Hz, 2H), 4.37 (m, 1H), 4.25 (s, 2H), 4.19 (dd, J=9.3, 6.4 Hz, 2H), 3.78 (dd, J=10.0, 4.9 Hz, 2H), 3.46 (t, J=6.1 Hz, 2H), 3.26 (d, J=6.1 Hz, 2H), 2.96 (s, 3H), 2.82 (m, 4H), 2.41 (q, J=7.6 Hz, 2H), 1.83 (m, 1H), 1.72 (d, J=12.4 Hz, 2H), 1.11 1.08 (t, J=7.6 Hz, 3H), 1.08 (m, 2H); MS (m/z) calculated for C₂₃H₃₄N₇O₃S⁺ (M+H⁺): 488.24. found 488.2.

By repeating the procedure described in the above example G1, using appropriate starting materials, the following compounds of Formula I, as identified in Table 4, are obtained.

TABLE 4

| Ex. # | Structure | NMR and/or ESMS |
|---|---|---|
| G2 | 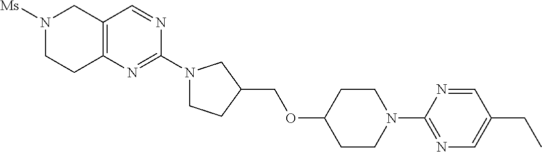 | MS calcd. for C₂₄H₃₆N₇O₃S [M + H]⁺: 502.3, found: 502.2. |
| G3 | 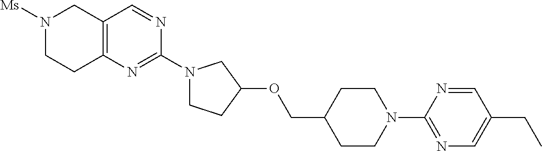 | MS calcd. for C₂₄H₃₆N₇O₃S [M + H]⁺: 502.3, found: 502.2. |
| G4 | 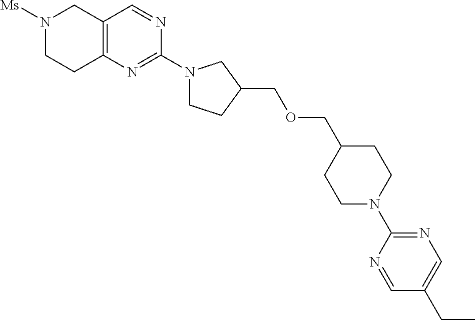 | MS calcd. for C₂₅H₃₈N₇O₃S [M + H]⁺: 516.3, found: 516.2. |
| G5 | 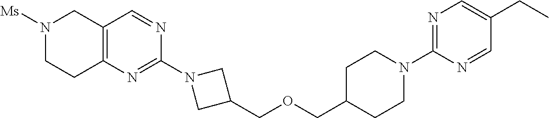 | MS calcd. for C₂₄H₃₆N₇O₃S [M + H]⁺: 502.3, found: 502.2. |
| G6 | 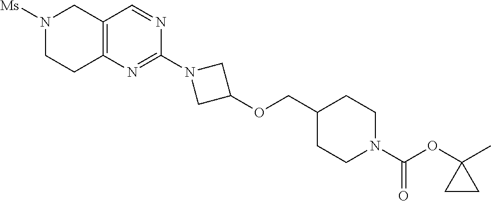 | ¹H NMR (400 MHz, dmso-d₆) δ = 8.22 (s, 1H), 4.37 (m, 1H), 4.35 (s, 2H), 4.20 (d, J = 6.4 Hz, 1H), 4.18 (d, J = 6.1 Hz, 1H), 3.9 (br, 2H), 3.79 (d, J = 4.0 Hz, 1H), 3.77 (d, J = 4.1 Hz, 1H), 3.45 (t, J = 6.0 Hz, 2H), 3.23 (d, J = 6.3 Hz, 2H), 2.96 (s, 3H), 2.79 (t, J = 6.0 Hz, 2H), 2.7 (br, 2H), 1.70 (m, 1H), 1.64 (d, J = 13.0 Hz, 2H), 1.46 (s, 3H), 1.02 (qd, J = 11.3, 2.4 Hz, 1H), 0.75 (m, 2H), 0.59 (m, 2H); MS calcd. for C₂₂H₃₄N₅O₅S [M + H]⁺: 480.2, found: 480.2. |
| G7 | 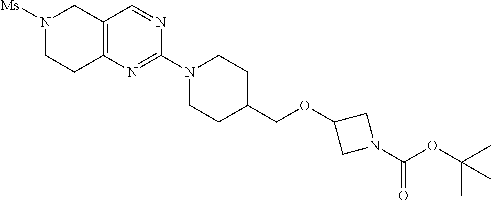 | MS calcd. For C₂₂H₃₅N₅NaO₅S [M + Na]⁺: 504.2, found: 504.2. |
| G8 | 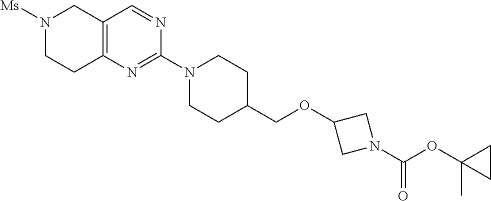 | MS calcd. for C₂₅H₃₆N₇O₃S [M + H]⁺: 514.3, found: 514.1. |
| G9 | 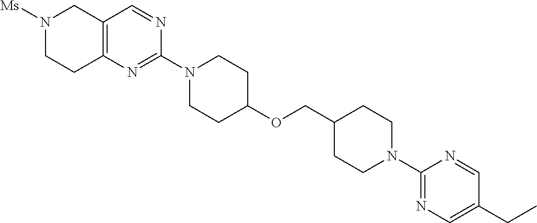 | MS calcd. for C₂₅H₃₈N₇O₃S [M + H]⁺: 516.3, found: 516.2. |
| G10 | 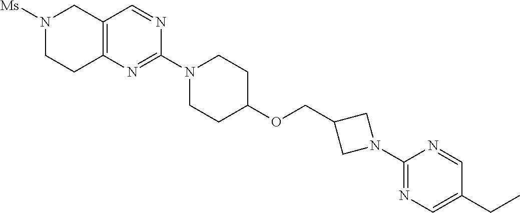 | MS calcd. for C₂₃H₃₄N₇O₃S [M + H]⁺: 488.2, found: 488.2. |
| G11 | 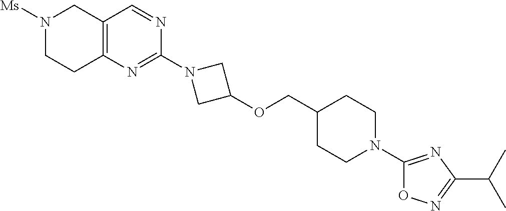 | MS calcd. for C₂₂H₃₄N₇O₄S [M + H]⁺: 492.2, found: 492.2. |
| G12 | 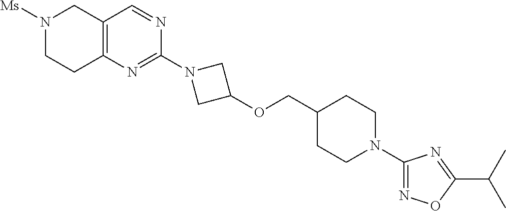 | MS calcd. for C₂₂H₃₄N₇O₄S [M + H]⁺: found: 492.2. |
| G13 | 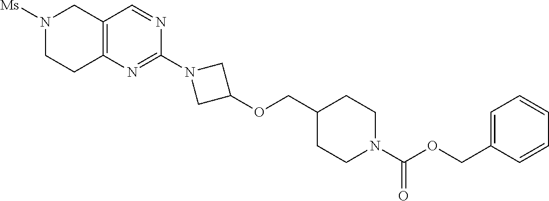 | MS calcd. for C₂₅H₃₄N₅O₅S [M + H]⁺: 516.2, found: 516.2. |
| G14 | 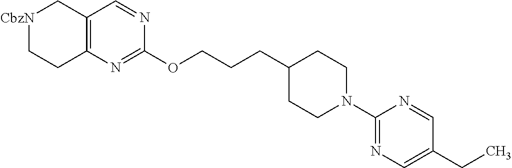 | ¹H NMR (400 MHz, CDCl₃) δ 8.38 (s, 2H), 7.84 (bs, 1H), 7.37 (m, 5H), 5.18 (s, 2H), 4.63 (m, 4H), 4.39 (m, 2H), 3.83 (m, 2H), 3.15 (t, J = 12.4 Hz, 2H), 2.90 (m, 2H), 2.59 (q, J = 7.6 Hz, 2H), 1.94 (d, J = 12.6 Hz, 2H), 1.85 (m, 2H), 1.69 (m, 1H), 1.48 (m, 2H), 1.27 (m, 2H), 1.25 (t, J = 7.5 Hz, 3H); MS calcd. for C₂₉H₃₇N₆O₃ [M + H]⁺: 517.6, found: 517.5. |
| G15 | 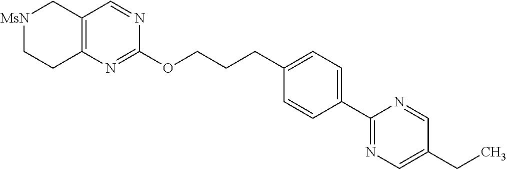 | ¹H NMR (400 MHz, CDCl₃) δ 8.63 (s, 2H), 8.33 (m, 3H), 7.33 (d, J = 8.5 Hz, 2H), 4.41 (s, 2H), 4.37 (t, J = 6.4 Hz, 2H), 4.25 (t, J = 6.3 Hz, 2H), 3.63 (m, 2H), 3.01 (m, 1H), 2.91 (s, 3H), 2.90 (m, 1H), 2.83 (m, 1H), 2.68 (m, 2H), 2.15 (m, 2H), 131 (t, J = 6.0 Hz, 3H); MS calcd. for C₂₃H₂₈N₄O₃S [M + H]⁺: 454.6, found: 454.4. |
| G16 | 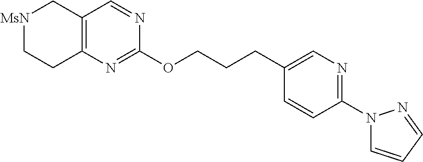 | ¹H NMR (400 MHz, d₆-DMSO) δ 8.58 (s, 1H), 8.45 (s, 1H), 8.34 (s, 1H), 7.89 (m, 2H), 7.80 (s, 1H), 6.56 (s, 1H), 4.35 (s, 2H), 4.29 (t, J = 6.4 Hz, 2H), 3.50 (t, J = 6.0 Hz, 2H), 2.98 (s, 3H), 2.90 (m, 2H), 2.79 (m, 2H), 2.07 (m, 2H); MS calcd. for C₁₉H₂₃N₆O₃S [M + H]⁺: 415.5, found: 415.4. |
| G17 | 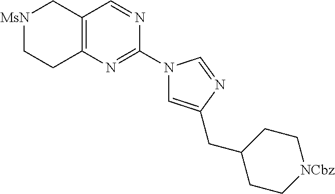 | MS calcd. for C₂₅H₃₁N₆O₄S [M + H]⁺: 511.2, found: 511.1. |
| G18 | 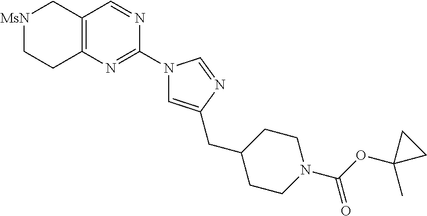 | ¹H NMR (400 MHz, d₄-MeOD) δ 9.78 (s, 1H), 8.74 (s, 1H), 8.23 (s, 1H), 4.60 (s, 2H), 4.07 (m, 2H), 3.72 (t, J = 6.0 Hz, 2H), 3.19 (t, J = 5.9 Hz, 2H), 3.01 (s, 3H), 2.79 (m, 4H), 1.89 (m, 1H), 1.73 (m, 2H), 1.51 (s, 3H), 1.19 (m, 2H), 0.83 (m, 2H), 0.63 (m, 2H); MS calcd. for C₂₂H₃₁N₆O₄S [M + H]⁺: 475.6, found: 475.4. |

### Intermediate 46: tert-Butyl 3-(methylsulfonyloxy)azetidine-1-carboxylate

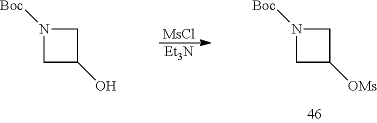

To a stirred solution of tert-butyl 3-hydroxyazetidine-1-carboxylate (1.0 g, 5.8 mmol) and triethylamine (1.6 mL, 11.6 mmol) in DCM (30 mL) is slowly added methanesulfonyl chloride (0.7 mL, 1.5 mmol) over 5 min at 0° C. The reaction is then stirred at rt for 2 h. The reaction mixture is partitioned between water (50 mL) and DCM (25 mL). The aqueous layer is separated and is further extracted with DCM (2×20 mL). The extractions are combined, dried (Na₂SO₄), and evaporated to give 46 as an amber-colored oil: ¹H NMR (400 MHz, CDCl₃) δ 5.20 (tt, J=6.7, 4.2 Hz, 1H), 4.28 (ddd, J=10.4, 6.7, 1.2 Hz, 2H), 4.10 (ddd, J=10.4, 4.2, 1.2 Hz, 2H), 3.07 (s, 3H), 1.44 (s, 9H); MS calcd. for C₉H₁₇NNaO₅S [M+Na]⁺: 274.1. found: 274.1.

### Intermediate 53: (±)-2-(4-(1-(Azetidin-3-yloxy)ethyl)piperidin-1-yl)-5-ethylpyrimidine hydrochloride

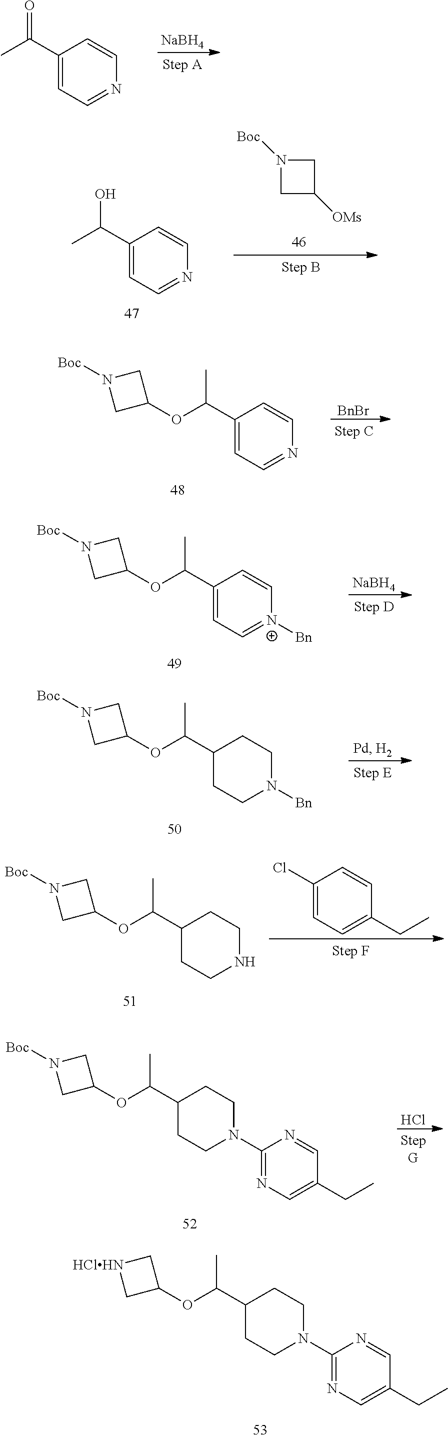

Step A: To a stirred solution of 4-acetylpyridine (1.0 mL, 9.0 mmol) in diethyl ether (25 mL) is added sodium borohydride (0.5 g, 13.2 mmol) and methanol (2 mL). The reaction is then stirred at rt for 18 h. The reaction mixture is concentrated to dryness, dissolved in dichloromethane and washed with sat. aqueous NH₄Cl. Drying over MgSO₄ and concentration yields (±)-1-(pyridin-4-yl)ethanol 47 as a colorless oil that slowly solidifies upon standing. ¹H NMR (400 MHz, CDCl₃) δ 8.52 (d, J=4.8 Hz, 2H), 7.31 (d, J=4.8 Hz, 2H), 4.91 (q, J=6.6 Hz, 1H), 2.64 (br, 1H), 1.50 (d, J=6.6 Hz, 3H).

Step B: tert-Butyl 3-(methylsulfonyloxy)azetidine-1-carboxylate 46 (1.87 g, 7.4 mmol), (±)-1-(pyridin-4-yl)ethanol 47 (1.1 g, 8.9 mmol) and tetra-n-butylammonium iodide (1.2 g, 3.2 mmol) are dissolved in dry dimethylformamide (10 mL). Sodium hydride (60% in mineral oil; 0.87 g, 21.8 mmol) is carefully added and the mixture is stirred in a preheated bath at 80° C. for 15 min. Cooling to rt, adding sat. NH₄Cl aqueous solution (2 mL) and extracting with dichloromethane (2×50 mL) are followed by washing with water (2×50 mL), drying over Na₂SO₄, and concentration. Purification by silicagel chromatography (0→100% EtOAc in hexane gradient) yielded (±)-tert-butyl 3-(1-(pyridin-4-yl)ethoxy)azetidine-1-carboxylate 48 as an oil. MS (m/z) calculated for C₁₅H₂₂N₂NaO₃⁺ (M+Na⁺): 301.2. found 301.2.

Step C: (±)-tert-Butyl 3-(1-(pyridin-4-yl)ethoxy)azetidine-1-carboxylate 48 (0.72 g, 2.6 mmol) in acetonitrile (5 mL) is treated with benzyl bromide (0.32 mL, 2.7 mmol) (as described in WO2003/076427, p. 52) and the mixture is stirred at 80° C. for 3 h. Concentration yields (±)1-benzyl-4-(1-(1-(tert-butoxycarbonyl)azetidin-3-yloxy)ethyl)pyridinium bromide 49 as a brown oil. MS (m/z) calculated for C₂₂H₂₉N₂O₃⁺ (M⁺): 369.2. found 369.2.

Step D: To a stirred solution of (±)1-benzyl-4-(1-(1-(tert-butoxycarbonyl)azetidin-3-yloxy)ethyl)pyridinium bromide 49 (from Step C above) in absolute ethanol (10 mL) is carefully added sodium borohydride (0.25 g, 6.6 mmol) (as described in WO2003/076427, p. 52). The reaction is then stirred at rt for 18 h. The reaction mixture is treated with sat. aqueous NH₄Cl solution (1 mL) and extracted with ethyl acetate (2×100 mL). The combined extracts were washed with sat. aqueous NaHCO₃ and brine, dried over Na₂SO₄ and concentrated to yield (±)-tert-butyl 3-(1-(1-benzylpiperidin-4-yl)ethoxy)azetidine-1-carboxylate 50 as a colorless oil. MS (m/z) calculated for C₂₂H₃₄N₂NaO₃⁺ (M+Na⁺): 397.3. found 397.2.

Step E: To a solution of (±)-tert-butyl 3-(1-(1-benzylpiperidin-4-yl)ethoxy)azetidine-1-carboxylate 50 (1.0 g, 2.6 mmol) in ethyl acetate (30 mL) and absolute ethanol (5 mL), palladium black (10% on carbon; 0.15 g, 0.14 mmol) is added. The mixture is degassed and vigorously stirred under 1 atm. of hydrogen for 48 h at rt. Filtration and concentration yields (±)-tert-butyl 3-(1-(piperidin-4-yl)ethoxy)azetidine-1-carboxylate 51 as a near-colorless oil. MS (m/z) calculated for C₁₅H₂₈N₂NaO₃⁺ (M+Na⁺): 307.2. found 307.2.

Step F: A solution of (±)-tert-butyl 3-(1-(piperidin-4-yl)ethoxy)azetidine-1-carboxylate 51 (40 mg, 0.16 mmol), Cs₂CO₃ (150 mg, 0.46 mol) and 2-chloro-5-ethylpyrimidine (40 mg, 0.28 mmol) in acetonitrile (3 mL) is stirred at 70° C. for 18 h. Concentration and purification by silicagel chromatography (0→100% EtOAc in hexane gradient) yields (±)-tert-butyl 3-(1-(1-(5-ethylpyrimidin-2-yl)piperidin-4-yl)ethoxy)azetidine-1-carboxylate 52 as a colorless oil: ¹H NMR (400 MHz, CDCl₃) δ=8.35 (s, 2H), 5.28 (m, 1H), 5.19 (m, 1H), 4.28 (m, 4H), 4.13 (m, 4H), 4.02 (m, 2H), 3.83 (m, 1H), 3.07 (br, 2H), 2.59 (q, J=7.6 Hz, 2H), 1.44 (m, 12H), 1.25 (t, J=7.6 Hz, 3H); MS calcd. for C₂₁H₃₄N₄NaO₃ [M+Na]⁺: 413.3. found: 413.2.

Step G: A solution of (±)-tert-butyl 3-(1-(1-(5-ethylpyrimidin-2-yl)piperidin-4-yl)ethoxy)azetidine-1-carboxylate 52 (40 mg, 0.1 mmol) in dichloromethane (5 mL) is treated with a solution of hydrogen chloride in diethyl ether (2M; 1 mL, 2 mmol) and stirred at rt for 18 h. Concentration yields (±)-2-(4-(1-(azetidin-3-yloxy)ethyl)piperidin-1-yl)-5-ethylpyrimidine hydrochloride 53 as a near-colorless oil. MS calcd. for C₁₆H₂₇N₄O [M+H]⁺: 291.2. found: 291.2.

### Example H1

### (±)-2-(3-(1-(1-(5-Ethylpyrimidin-2-yl)piperidin-4-yl)ethoxy)azetidin-1-yl)-6-(methylsulfonyl)-5,6,7,8-tetrahydropyrido[4,3-d]pyrimidine

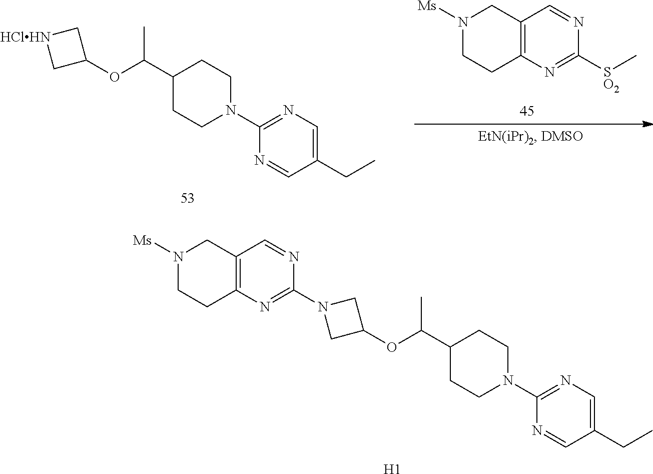

2,6-Bis(methylsulfonyl)-5,6,7,8-tetrahydropyrido[4,3-d]pyrimidine 45 (0.03 g, 1.0 mmol), (±)-2-(4-(1-(azetidin-3-yloxy)ethyl)piperidin-1-yl)-5-ethylpyrimidine hydrochloride 53 (0.03 g, 1.0 mmol), and ethyldiisopropyl amine (0.25 mL, 1.5 mmol) are dissolved in DMSO (3 mL) and heated to 65° C. for 6 h. Cooling to rt and purification by reversed-phased HPLC (5→100% ACN in water gradient using TFA as an ion-pairing reagent) yields (±)-2-(3-(1-(1-(5-ethylpyrimidin-2-yl)piperidin-4-yl)ethoxy)azetidin-1-yl)-6-(methylsulfonyl)-5,6,7,8-tetrahydropyrido[4,3-d]pyrimidine F1 as a white solid. MS (m/z) calculated for C₂₄H₃₆N₇O₃S⁺ (M+H⁺): 502.3. found 502.2.

### Intermediate 57: 5-(Azetidin-3-yloxy)-2-(5-ethylpyrimidin-2-yl)-1,2,3,4-tetrahydroisoquinoline hydrochloride

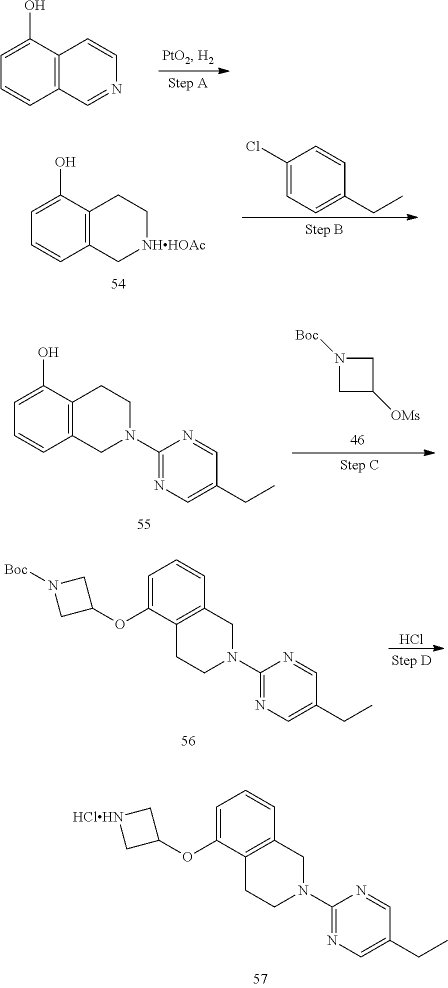

Step A: Isoquinolin-5-ol (3.2 g, 22 mmol) in glacial acetic acid (25 mL) is treated with platinum dioxide (0.15 g, 0.7 mmol). The reaction is degassed and shaken at rt for 18 h under 40 psi positive hydrogen pressure (as described in J. Org. Chem. 1962, 4571). Filtration and concentration to dryness and treatment with chloroform (1 mL) slowly yields a white solid. Concentration, suspension in diethyl ether (150 mL), filtration, washing with more diethyl ether and air-drying yields 1,2,3,4-tetrahydroisoquinolin-5-ol acetate 54 as a white solid. ¹H NMR (400 MHz, dmso-d₆) δ 9.2 (br, 2H), 6.89 (dd, J=7.9, 7.5 Hz, 1H), 6.59 (d, J=7.9 Hz, 1H), 6.45 (d, J=7.5 Hz, 1H), 3.78 (s, 2H), 3.5 (br, 1H), 2.94 (d, J=6.0 Hz, 2H), 2.49 (d, J=6.0 Hz, 2H), 1.89 (s, 3H); MS (m/z) calculated for C₉H₁₂NO⁺ (M+Na⁺): 150.2. found 150.1.

Step B: 1,2,3,4-Tetrahydroisoquinolin-5-ol acetate 54 (0.45 g, 2.2 mmol), 2-chloro-5-ethylpyrimidine (0.3 g, 2.1 mmol), and powdered cesium carbonate (1.85 g, 5.7 mmol) are stirred in dimethylacetamide (10 mL) at 70° C. for 18 h. Cooling to rt, adding ethyl acetate (2×50 mL) are followed by washing with water (2×50 mL), drying over Na₂SO₄, and concentration yields an oil. Purification by silicagel chromatography (0→80% EtOAc in hexane gradient) yields 2-(5-ethylpyrimidin-2-yl)-1,2,3,4-tetrahydroisoquinolin-5-ol 55 as an oil. ¹H NMR (400 MHz, dmso-d₆) δ 9.38 (s, 1H), 8.26 (s, 2H), 6.98 (t, J=7.8 Hz, 1H), 6.65 (t, J=7.8 Hz, 2H), 4.77 (s, 2H), 3.95 (t, J=6.0 Hz, 2H), 2.67 (d, J=6.0 Hz, 2H), 2.44 (q, J=7.6 Hz, 2H), 1.13 (t, J=7.6 Hz, 3H); MS (m/z) calculated for C₁₅H₁₈N₃O⁺ (MAT): 256.2. found 256.2.

Step C: tert-Butyl 3-(methylsulfonyloxy)azetidine-1-carboxylate 46 (0.11 g, 0.4 mmol), -(5-ethylpyrimidin-2-yl)-1,2,3,4-tetrahydroisoquinolin-5-ol 55 (0.12 g, 0.48 mmol) and powdered cesium carbonate (0.45 g, 1.4 mmol) are dissolved in dry acetonitrile (5 mL). The mixture is stirred at 65° C. for 18 h. Cooling to rt, filtration and concentration yields tert-butyl 3-(2-(5-ethylpyrimidin-2-yl)-1,2,3,4-tetrahydroisoquinolin-5-yloxy)azetidine-1-carboxylate 56 as an oil. ¹H NMR (400 MHz, CDCl₃) δ 8.23 (s, 2H), 7.11 (dd, J=7.7, 8.0 Hz, 1H), 6.87 (d, J=7.7 Hz, 1H), 6.33 (d, J=8.0 Hz, 1H), 4.89 (s, 2H), 4.67 (m, 1H), 4.30 (m, 2H), 4.06 (t, J=6.0 Hz, 2H), 4.00 (dd, J=4.2, 10.0 Hz, 2H), 2.88 (t, J=6.0 Hz, 2H), 2.48 (q, J=7.6 Hz, 2H), 1.45 (s, 9H), 1.20 (t, J=7.6 Hz, 3H); MS (m/z) calculated for C₂₃H₃₀N₄NaO₃⁺ (M+Na⁺): 433.3. found 433.2.

Step D: A solution of tert-butyl 3-(2-(5-ethylpyrimidin-2-yl)-1,2,3,4-tetrahydroisoquinolin-5-yloxy)azetidine-1-carboxylate 56 (0.16 g, 0.4 mmol) in dichloromethane (4 mL) is treated with a solution of hydrogen chloride in diethyl ether (2M; 2 mL, 4 mmol) and stirred at rt for 18 h. Concentration yields 5-(azetidin-3-yloxy)-2-(5-ethylpyrimidin-2-yl)-1,2,3,4-tetrahydroisoquinoline hydrochloride 57 as a near-colorless thick oil. MS calcd. for C₁₈H₂₃N₄O [M+H]⁺: 311.2. found: 311.2.

### Example I1

### 2-(3-(2-(5-Ethylpyrimidin-2-yl)-1,2,3,4-tetrahydroisoquinolin-5-yloxy)azetidin-1-yl)-6-(methylsulfonyl)-5,6,7,8-tetrahydropyrido[4,3-d]pyrimidine

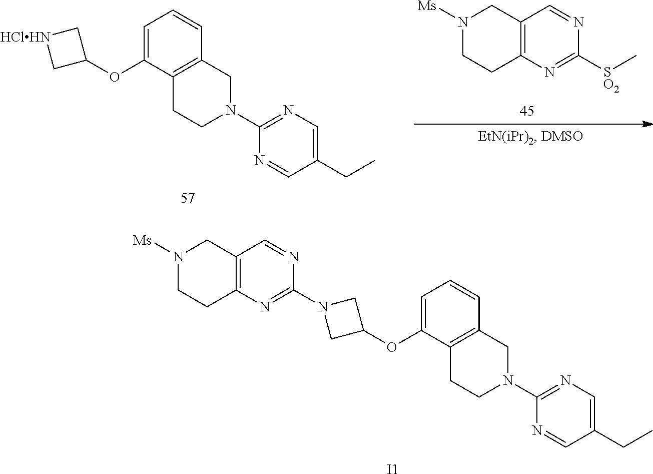

2,6-Bis(methylsulfonyl)-5,6,7,8-tetrahydropyrido[4,3-d]pyrimidine 45 (0.06 g, 0.2 mmol), 5-(azetidin-3-yloxy)-2-(5-ethylpyrimidin-2-yl)-1,2,3,4-tetrahydroisoquinoline hydrochloride 53 (0.07 g, 0.2 mmol), and ethyldiisopropyl amine (0.1 mL, 0.6 mmol) are dissolved in DMSO (2 mL) and heated to 65° C. for 4 h. Cooling to rt and purification by reversed-phased HPLC (5→100% ACN in water gradient using TFA as an ion-pairing reagent) yields 2-(3-(2-(5-ethylpyrimidin-2-yl)-1,2,3,4-tetrahydroisoquinolin-5-yloxy)azetidin-1-yl)-6-(methylsulfonyl)-5,6,7,8-tetrahydropyrido[4,3-d]pyrimidine I1 as a white solid. MS (m/z) calculated for C₂₆H₃₂N₇O₃S⁺ (M+H⁺): 522.3. found 522.2.

Intermediate 64: (±)-(3R,45)-1-methylcyclopropyl 4-((azetidin-3-yloxy)methyl)-3-methoxypiperidine-1-carboxylate hydrochloride

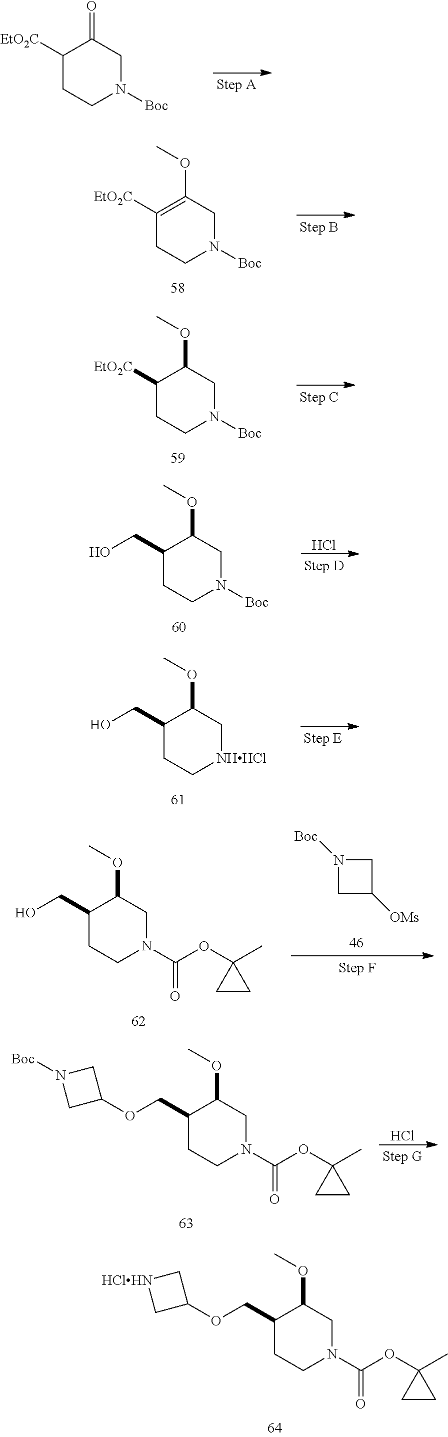

Step A: A solution of 1-tert-butyl 4-ethyl 3-oxopiperidine-1,4-dicarboxylate (5.5 g, 20.3 mmol) in a mixture of dichloromethane and methanol (25 mL of a 95:5 mixture) is treated with a solution of TMS-diazomethane in diethyl ether (15.5 ml of a 2 M solution). The reaction is stirred at 50° C. with a reflux condenser for a week. Another 5-mL portion of TMS-diazomethane solution is added after days 2 and 5. The reaction is cooled to room temperature and quenched by addition of acetic acid. The solvents are then removed and the residue is purified by silica gel using a linear gradient of 0-50% ethyl acetate in hexane to afford 1-tert-butyl 4-ethyl 3-methoxy-5,6-dihydropyridine-1,4(2H)-dicarboxylate 58; ¹H NMR (CDCl₃, 400 MHz): δ 4.20 (dd, J=7.2, 14.3, 1H), 4.08 (m, 2H), 3.78 (s, 3H), 3.43 (m, 2H), 2.41 (m, 2H), 1.47 (s, 9H), 1.29 (dd, J=7.2, 7.2, 1H); ESIMS m/z for (M+H)⁺ C₁₉H₃₃N₅O₅S calcd.: 442.2. found: 442.3.

Step B: A solution of 58 (1 g, 3.5 mmol) in methanol (15 mL) is treated with 10% Pd/C (150 mg) and hydrogenated at 50 psi overnight. The catalyst is removed by filtration and the residue is purified on silica gel using 0-100% ethyl acetate in hexane to afford (±)-(3R,4R)-1-tert-butyl 4-ethyl 3-methoxypiperidine-1,4-dicarboxylate 59; ESIMS m/z for (M−tBu+H)⁺ C₁₀H₁₈NO₅ calcd.: 232.1. found: 232.1.

Step C: A sample of 59 (600 mg, 2.1 mmol) is treated with 2 M LiBH₄ in tetrathydrofuran (5 mL, 10 mmol) and heated to reflux overnight. The reaction is cooled to room temperature and then treated with saturated aqueous ammonium chloride solution. The reaction is then diluted with ethyl acetate and the organics are isolated, dried over MgSO₄, filtered, evaporated and purified by silica gel column chromatography using a linear gradient of 0-100% ethyl acetate in hexane to afford (±)-(3R,4S)-tert-butyl 4-(hydroxymethyl)-3-methoxypiperidine-1-carboxylate 60; ESIMS m/z for (M−tBu+H)⁺ C₈H₁₆NO₄ calcd.: 190.1. found: 190.1.

Step D: A solution of (±)-(3R,4S)-tert-butyl 4-(hydroxymethyl)-3-methoxypiperidine-1-carboxylate 60 (0.18 g, 0.7 mmol) in dichloromethane (3 mL) is treated with a solution of hydrogen chloride in diethyl ether (2M; 1 mL, 2 mmol) and stirred at rt for 18 h. Concentration yields (±)-((3R,4S)-3-methoxypiperidin-4-yl)methanol hydrochloride 61 as a near-colorless thick oil. MS calcd. for C₇H₁₆NO [M+H]⁺: 146.1. found: 146.0.

Step E: (±)-((3R,45)-3-Methoxypiperidin-4-yl)methanol hydrochloride 61 (0.13 g, 0.7 mmol) and 1-methylcyclopropyl 4-nitrophenyl carbonate 21 (0.2 mg, 0.8 mmol) are dissolved in dichloromethane (3 mL). Triethylamine (0.35 mL, 2.5 mmol) is added and the reaction mixture is stirred at rt overnight. It is then diluted with dichloromethane and washed with 1M NaOH (4×). The organic phase is then washed with 1M HCl (1×) and brine (1×), dried (Na₂SO₄) and concentrated to afford (±)-(3R,45)-1-methylcyclopropyl 4-(hydroxymethyl)-3-methoxypiperidine-1-carboxylate 62. MS calcd. for C₁₂H₂₂NO₄ [M+H⁺] 244.1. found 244.1.

Step C: (±)-(3R,45)-1-methylcyclopropyl 4-(hydroxymethyl)-3-methoxypiperidine-1-carboxylate 62 (0.17 g, 0.7 mmol), tert-butyl 3-(methyl sulfonyloxy)azetidine-1-carboxylate (0.2 g, 0.8 mmol) and tetra-n-butylammonium iodide (0.15 g, 0.4 mmol) are dissolved in dry dimethylformamide (2 mL). Sodium hydride (60% in mineral oil; 0.2 g, 5.4 mmol) is carefully added and the mixture is stirred in a preheated bath at 80° C. for 15 min. Cooling to rt, adding sat. NH₄Cl aqueous solution (2 mL) and extracting with dichloromethane (2×50 mL) are followed by washing with water (2×50 mL), drying over Na₂SO₄, and concentration. Purification by silicagel chromatography (0→100% EtOAc in hexane gradient) yielded (±)-(3R,45)-1-methylcyclopropyl 4-((1-(tert-butoxycarbonyl)azetidin-3-yloxy)methyl)-3-methoxypiperidine-1-carboxylate 63 as an oil. MS (m/z) calculated for C₂₀H₃₄N₂NaO₆⁺ (M+Na⁺): 421.3. found 421.2.

Step D: A solution of (±)-(3R,4S)-1-methylcyclopropyl 4-((1-tert-butoxycarbonyl)azetidin-3-yloxy)methyl)-3-methoxypiperidine-1-carboxylate 63 (0.22 g, 0.6 mmol) in dichloromethane (2 mL) is treated with a solution of hydrogen chloride in diethyl ether (2M; 1 mL, 2 mmol) and stirred at rt for 18 h. Concentration yields (±)-(3R,4S)-1-methylcyclopropyl 4-((azetidin-3-yloxy)methyl)-3-methoxypiperidine-1-carboxylate hydrochloride 64 as a near-colorless thick oil. MS calcd. for C₁₅H₂₇N₂O₄ [M+H]⁺: 299.2. found: 299.2.

### Example J1

### (±)-(3R,45)-1-Methylcyclopropyl 3-methoxy-4-((1-(6-(methylsulfonyl)-5,6,7,8-tetrahydropyrido[4,3-d]pyrimidin-2-yl)azetidin-3-yloxy)methyl)piperidine-1-carboxylate

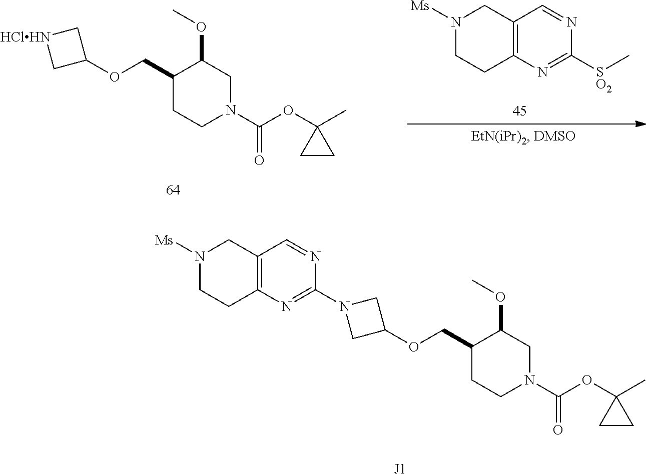

2,6-Bis(methylsulfonyl)-5,6,7,8-tetrahydropyrido[4,3-d]pyrimidine 45 (0.06 g, 0.2 mmol), (±)-(3R,45)-1-methylcyclopropyl 4-((azetidin-3-yloxy)methyl)-3-methoxypiperidine-1-carboxylate hydrochloride 64 (0.07 g, 0.2 mmol), and ethyldiisopropyl amine (0.1 mL, 0.6 mmol) are dissolved in DMSO (2 mL) and heated to 75° C. for 4 h. Cooling to rt and purification by reversed-phased HPLC (5→100% ACN in water gradient using TFA as an ion-pairing reagent) yields (±)-(3R,4S)-1-methylcyclopropyl 3-methoxy-4-(1-(6-(methylsulfonyl)-5,6,7,8-tetrahydropyrido[4,3-d]pyrimidin-2-yl)azetidin-3-yloxy)methyl)piperidine-1-carboxylate J1 as a white solid. MS (m/z) calculated for C₂₃H₃₆N₅O₆S⁺ (M+H⁺): 510.3. found 510.2.

By repeating the procedure described in the above example J1, using appropriate starting materials, the following compounds of Formula I, as identified in Table 5, are obtained.

TABLE 5

| Ex. # | Structure | NMR and/or ESMS |
|---|---|---|
| J2 | 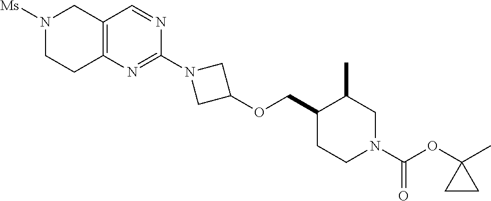 | MS calcd. for C₂₃H₃₆N₅O₅S [M + H]⁺: 494.2, found: 494.2. |
| J3 | 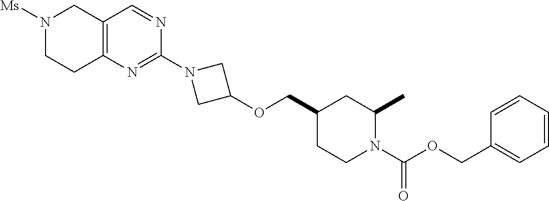 | MS calcd. for C₂₆H₃₆N₅O₅S [M + H]⁺: 530.2, found: 530.1. |

### Biological Assays

Generation of Stable Cell Line

Flp-In-CHO cells (Invitrogen, Cat.# R758-07) are maintained in Ham's F12 medium supplemented with 10% fetal bovine serum, 1% antibiotic mixture and 2 mM L-glutamine. The cells are transfected with a DNA mixture containing human GPR119 in pcDNA5/FRT vector and the pOG44 vector (1:9) using Fugene6 (Roche), according to the manufacturer's instruction. After 48 hours, the medium is changed to medium supplemented with 400 μg/ml hygromycin B to initiate the selection of stably transfected cells.

Cyclic AMP Assay in Stable Cell Line

To test the activity of compounds of the invention, Flp-In-CHO-hGPR119 cells are harvested and resuspended in DMEM plus 3% lipid-depleted fetal bovine serum. Forth μl of cells are plated in 384 well plates at a density of 15,000 cells/well. IBMX (3-isobutyl-1-methyl-xanthine) is added to the cells to a final concentration of 1 mM, followed by the addition of 500 nl of the compound to be tested. The cells are incubated at 37° C. for 30 minutes. Equal volume (20 μl) of the HTRF reagents, anti-cAMP-Cryptate and cAMP-XL665, are added to the cells. The plates are incubated at room temperature for 1 hour and read on a HTRF reader according to the manufacturer's instruction.

Compounds of Formula I, in free form or in pharmaceutically acceptable salt form, produced a concentration-dependent increase in intracellular cAMP level. Compound of the invention show an EC₅₀ of between 1×10⁻⁵ and 1×10⁻¹⁰M, preferably less than 500 nM, more preferably less than 100 nM. For example, compounds of the invention show EC₅₀s according to the following table:

| Example Number | hGPR119 EC₅₀ (nM) |
|---|---|
| A4 | 3705 |
| A5 | 4360 |
| A7 | 94 |
| A8 | 137 |
| A9 | 191 |
| B3 | 2435 |
| C1 | 514 |
| D2 | 2 |
| D3 | 4 |
| D4 | 6 |
| D5 | 7 |
| D7 | 8 |
| D8 | 10 |
| D9 | 5 |
| E1 | 212 |
| F1 | 27 |
| G1 | 9 |
| G5 | 666 |
| G6 | 28 |
| G7 | 22 |
| G8 | 105 |
| G9 | 317 |
| G11 | 170 |
| G15 | 366 |
| G16 | 1025 |
| G18 | 122 |
| H1 | 183 |
| I1 | 49 |
| J1 | 711 |
| J2 | 174 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 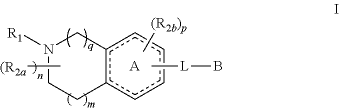
in which:
A is a 6 member saturated, partially unsaturated or aromatic ring system containing at least one heteroatom or moiety selected from N and C(O);
B is selected from C₆₋₁₀aryl, C₁₋₁₀heteroaryl, C₃₋₁₂cycloalkyl and C₁₋₈ heterocycloalkyl; wherein said aryl, heteroaryl, cycloalkyl or heterocycloalkyl is substituted with one to three R₃ radicals;
n is selected from 0, 1, 2 and 3;
P is selected from 0, 1 and 2;
q is selected from 0 and 1;
m is selected from 1 and 2;
L is selected from a bond, C₁₋₆alkylene, —X₁OX₂—, —X₁NR₄X₂—, —OX₃O— and —X₆X₂—; wherein R₄ is selected from hydrogen and C₁₋₄alkyl; X₁ is selected from a bond, C₁₋₄alkylene and C₃₋₈heterocycloalkyl-C₀₋₁alkyl; X₂ is selected from a bond and C₁₋₄alkylene; X₃ is C₁₋₄alkylene; and X₆ is a 5 member heteroaryl;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆ alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;
R₂ₐ and R_{2b} are independently selected from halo, cyano, hydroxy, C₁₋₄alkyl, amino, nitro, —C(O)OR₅ₑ, —C(O)R₅ₑ, and —NR₅ₑR_{5f}; wherein R₅ₑ and R_{5f} are independently selected from hydrogen, C₁₋₆ alkyl, C₃₋₁₂cycloalkyl, halo-substituted-C₁₋₆alkyl, halo-substituted-C₁₋₆cycloalkyl, C₆₋₁₀ aryl and C₁₋₁₀heteroaryl; wherein said aryl or heteroaryl of R₅ₑ or R_{5f} can be optionally substituted with 1 to 3 radicals independently selected from C₁₋₆alkyl, C₁₋₆ alkoxy, halo-substituted-C₁₋₆ alkyl and halo-substituted-C₁₋₆alkoxy;
R₃ is selected from C₁₋₁₀heteroaryl, C₆₋₁₀aryl, C₃₋₈heterocycloalkyl, halo, —C(O)OR₆ₐ, —C(O)R₆ₐ, —S(O)₀₋₂R₆ₐ, —C(O)R₇, —C(O)X₅NR₆ₐC(O)OR_{6b}, —C(S)OR₆ₐ, —C(S)R₆ₐ, —C(S)R₇ and —C(S)X₅NR₆ₐC(O)OR_{6b}; wherein X₅ is selected from a bond and C₁₋₆alkylene; or two adjacent R₃ groups together with the carbon atom to which they are attached form a C₃₋₈heterocycloalkyl optionally substituted with a group selected from —C(O)OR_{6c} and —R_{6d}; R₆ₐ, R_{6b} and R_{6c} are independently selected from hydrogen, C₁₋₆alkyl, halo-substituted-C₁₋₆alkyl, C₃₋₁₂cycloalkyl optionally substituted with C₁₋₄ alkyl, halo-substituted-C₁₋₆cycloalkyl; R_{6d} is C₁₋₁₀heteroaryl optionally substituted with C₁₋₄alkyl; R₇ is selected from C₁₋₈alkyl, C₃₋₈cycloalkyl, C₆₋₁₀aryl, C₁₋₁₀heteroaryl, halo-substituted C₁₋₈alkyl, halo-substituted-C₃₋₈cycloalkyl, halo-substituted-C₆₋₁₀aryl and halo-substituted-C₆₋₁₀heteroaryl; wherein said aryl, heteroaryl or heterocycloalkyl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐC(O)R₈ₐ, —X₅ₐR₉, C₁₋₆alkyl, C₁₋₆alkoxy, halo-substituted-C₁₋₆alkyl and halo-substituted-C₁₋₆alkoxy; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀ aryl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; or the pharmaceutically acceptable salts thereof.

2. The compound of claim 1 selected from Formula Ia, Ib, Ic, Id and Ie: 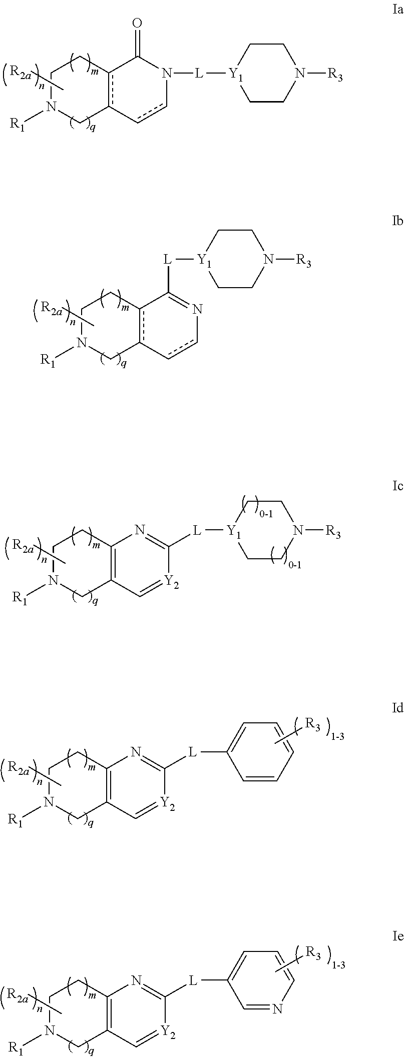
in which:
n is selected from 0, 1, 2 and 3;
q is selected from 0 and 1;
m is selected from 1 and 2;
L is selected from a bond, C₁₋₆alkylene, —X₁OX₂—, —X₁NR₄X₂—, —OX₃O— and —X₆X₂—; wherein R₄ is selected from hydrogen and C₁₋₄alkyl; X₁ is selected from a bond, C₁₋₄alkylene and C₃₋₈heterocycloalkyl-C₀₋₁alkyl; X₂ is selected from a bond and C₁₋₄alkylene; X₃ is C₁₋₄alkylene; and X₆ is a 5 member heteroaryl;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;
R₂ₐ is selected from halo, cyano, hydroxy, C₁₋₄alkyl, amino, nitro, —C(O)OR₅ₑ, —C(O)R₅ₑ and —NR₅ₑR_{5f}; wherein R₅ₑ and R_{5f} are independently selected from hydrogen, C₁₋₆alkyl, C₃₋₁₂cycloalkyl, halo-substituted-C₁₋₆alkyl, halo-substituted-C₁₋₆cycloalkyl, C₆₋₁₀ aryl and C₁₋₁₀heteroaryl; wherein said aryl or heteroaryl of R₅ₑ or R_{5f} can be optionally substituted with 1 to 3 radicals independently selected from C₁₋₆alkyl, C₁₋₆alkoxy, halo-substituted-C₁₋₆ alkyl and halo-substituted-C₁₋₆alkoxy;
R₃ is selected from C₁₋₁₀heteroaryl, C₆₋₁₀aryl, C₃₋₈heterocycloalkyl, halo, —C(O)OR₆ₐ, —C(O)R₆ₐ, —S(O)₀₋₂R₆ₐ, —C(O)R₇, —C(O)X₅NR₆ₐC(O)OR_{6b}, —C(S)OR₆ₐ, —C(S)R₆ₐ, —C(S)R₇ and —C(S)X₅NR₆ₐC(O)OR_{6b}; wherein X₅ is selected from a bond and C₁₋₆alkylene; or two adjacent R₃ groups together with the carbon atom to which they are attached form a C₃₋₈heterocycloalkyl optionally substituted with a group selected from —C(O)OR_{6c} and —R_{6d}; R₆ₐ, R_{6b} and R_{6c} are independently selected from hydrogen, C₁₋₆alkyl, halo-substituted-C₁₋₆alkyl, C₃₋₁₂cycloalkyl optionally substituted with C₁₋₄ alkyl, halo-substituted-C₁₋₆cycloalkyl; R_{6d} is C₁₋₁₀heteroaryl optionally substituted with C₁₋₄alkyl; R₇ is selected from C₁₋₈alkyl, C₃₋₈cycloalkyl, C₆₋₁₀aryl, C₁₋₁₀heteroaryl, halo-substituted C₁₋₈alkyl, halo-substituted-C₃₋₈cycloalkyl, halo-substituted-C₆₋₁₀aryl and halo-substituted-C₆₋₁₀heteroaryl; wherein said aryl, heteroaryl or heterocycloalkyl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐC(O)R₈ₐ, —X₅ₐR₉, C₁₋₆alkyl, C₁₋₆alkoxy, halo-substituted-C₁₋₆alkyl and halo-substituted-C₁₋₆alkoxy; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀ aryl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; and
Y₁ and Y₂ are independently selected from CH and N; wherein the dotted lines of formulae Ia or Ib independently indicate the presence of a double or single bond.

3. The compound of claim 2 in which L is selected from a bond, —(CH₂)₁₋₄—, —O(CH₂)₀₋₄—, —CH₂NH(CH₂)₀₋₂—, —NH(CH₂)₁₋₃—, —N(CH₃)(CH₂)₁₋₃—, —CH₂O(CH₂)₁₋₂—, —O(CH₂)₂O— and —X₆(CH₂)₀₋₁; wherein X₆ is imidazole; or a moiety of formula II:

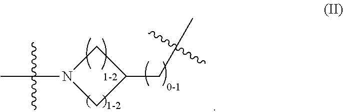

4. The compound of claim 3 in which R₁ is selected from methyl-sulfonyl, butyl-sulfonyl, phenyl-sulfonyl, isopropyl-sulfonyl, ethyl-sulfonyl, ethenyl-sulfonyl, isopropoxy-carbonyl, benzoxy-carbonyl, ethoxy-carbonyl, methoxy-carbonyl, t-butoxy-carbonyl and trifluoromethyl-sulfonyl.

5. The compound of claim 4 in which R₃ is selected from halo, t-butoxy-carbonyl, t-butoxy-carbonyl-amino-methyl, isopropoxy-carbonyl, 3-isopropyl-(1,2,4-oxadiazol-5-yl), (1-methylcyclopropoxy)carbonyl, azetidin-1-yl, pyridinyl, piperidinyl, pyrimidinyl, pyrazolyl, benzoxycarbonyl and cyclopropoxy-carbonyl; wherein said azetidin-1-yl, pyridinyl, piperidinyl, cyclopropoxy or pyrimidinyl can be optionally substituted by 1 to 2 radicals independently selected from methyl, isopropyl, ethyl and pyrimidinyl optionally substituted with ethyl; or two adjacent R₃ groups together with the carbon atom to which they are both attached form 1-(tert-butoxycarbonyl)piperidin-4-yl.

6. The compound of claim 1 selected from: Isopropyl 4-(3-(1,2,3,4-tetrahydro-2-methanesulfonyl-5-oxo-2,6-naphthyridin-6(5H)-yl)propyl)piperidine-1-carboxylate; isopropyl 4-(3-(1,2,3,4-tetrahydro-2-methanesulfonyl-2,6-naphthyridin-5-yloxy)propyl)piperidine-1-carboxylate; isopropyl 4-(3-(1,2,3,4,4a,7,8,8a-octahydro-2-methanesulfonyl-2,6-naphthyridin-5-yloxy)propyl)piperidine-1-carboxylate; isopropyl 4-(6-(methylsulfonyl)-5,6,7,8-tetrahydro-2,6-naphthyridin-1-yloxy)piperidine-1-carboxylate; isopropyl 4-(6-(methylsulfonyl)-1-oxooctahydro-2,6-naphthyridin-2(1H)-yl)piperidine-1-carboxylate; isopropyl 4-((6-(methylsulfonyl)-1-oxo-5,6,7,8-tetrahydro-2,6-naphthyridin-2(1H)-yl)methyl)piperidine-1-carboxylate; isopropyl 4-(4-(6-(methylsulfonyl)-1-oxo-5,6,7,8-tetrahydro-2,6-naphthyridin-2(1H)-yl)butyl)piperidine-1-carboxylate; isopropyl 4-(4-(6-(methylsulfonyl)-3,4,4a,5,6,7,8,8a-octahydro-2,6-naphthyridin-1-yloxy)butyl)piperidine-1-carboxylate; isopropyl 4-(4-(6-(methylsulfonyl)-5,6,7,8-tetrahydro-2,6-naphthyridin-1-yloxy)butyl)piperidine-1-carboxylate; tert-Butyl 4-(((6-(methylsulfonyl)-5,6,7,8-tetrahydropyrido[4,3-d]pyrimidin-2-yl)methylamino)methyl)piperidine-1-carboxylate; tert-butyl 4-(2-((6-(methylsulfonyl)-5,6,7,8-tetrahydropyrido[4,3-d]pyrimidin-2-yl)methylamino)ethyl)piperidine-1-carboxylate; 2-(3-bromophenyl)-N-((6-(methylsulfonyl)-5,6,7,8-tetrahydropyrido[4,3-d]pyrimidin-2-yl)methyl)ethanamine; tert-butyl 4-((6-(methylsulfonyl)-5,6,7,8-tetrahydropyrido[4,3-d]pyrimidin-2-yl)methylamino)benzylcarbamate; 1-Methylcyclopropyl 4-(2-((6-(methylsulfonyl)-5,6,7,8-tetrahydropyrido[4,3-d]pyrimidin-2-yl)methoxy)ethyl)piperidine-1-carboxylate; 3-Isopropyl-5-(4-(3-(6-(methylsulfonyl)-5,6,7,8-tetrahydropyrido[4,3-d]pyrimidin-2-yloxy)propyl)piperidin-1-yl)-1,2,4-oxadiazole; 1-Methylcyclopropyl 4-(3-(6-(methylsulfonyl)-5,6,7,8-tetrahydropyrido[4,3-d]pyrimidin-2-yloxy)propyl)piperidine-1-carboxylate; 2-(3-(1-(5-Ethylpyrimidin-2-yl)piperidin-4-yl)propoxy)-6-(methylsulfonyl)-5,6,7,8-tetrahydropyrido[4,3-d]pyrimidine; N-(3-(1-(3-isopropyl-1,2,4-oxadiazol-5-yl)piperidin-4-yl)propyl)-6-(methylsulfonyl)-5,6,7,8-tetrahydropyrido[4,3-d]pyrimidin-2-amine; N-(3-(1-(5-Ethylpyrimidin-2-yl)piperidin-4-yl)propyl)-6-(methylsulfonyl)-5,6,7,8-tetrahydropyrido[4,3-d]pyrimidin-2-amine; N-(3-(1-(5-Ethylpyrimidin-2-yl)piperidin-4-yl)propyl)-N-methyl-6-(methylsulfonyl)-5,6,7,8-tetrahydropyrido[4,3-d]pyrimidin-2-amine; 1-methylcyclopropyl 4-(3-(6-(methylsulfonyl)-5,6,7,8-tetrahydropyrido[4,3-d]pyrimidin-2-ylamino)propyl)piperidine-1-carboxylate; 1-methylcyclopropyl 4-(3-(methyl(6-(methylsulfonyl)-5,6,7,8-tetrahydropyrido[4,3-d]pyrimidin-2-yl)amino)propyl)piperidine-1-carboxylate; 2-(2-(1-(5-ethylpyrimidin-2-yl)piperidin-4-yloxy)ethoxy)-6-(methylsulfonyl)-5,6,7,8-tetrahydropyrido[4,3-d]pyrimidine; 2-(3-(1-(5-Ethylpyrimidin-2-yl)piperidin-4-yl)propoxy)-6-(methylsulfonyl)-5,6,7,8-tetrahydro-1,6-naphthyridine; 5-ethyl-2-(4-{[(2S)-1-{6-methanesulfonyl-5H,6H,7H,8H-pyrido[4,3-d]pyrimidin-2-yl}pyrrolidin-2-yl]methoxy}piperidin-1-yl)pyrimidine; benzyl 4-[(1-{6-methanesulfonyl-5H,6H,7H,8H-pyrido[4,3-d]pyrimidin-2-yl}-1H-imidazol-4-yl)methyl]piperidine-1-carboxylate; 1-methylcyclopropyl 3-[(1-{6-methanesulfonyl-5H,6H,7H,8H-pyrido[4,3-d]pyrimidin-2-yl}piperidin-4-yl)methoxy]azetidine-1-carboxylate; 5-[3-({6-methanesulfonyl-5H,6H,7H,8H-pyrido[4,3-d]pyrimidin-2-yl}oxy)propyl]-2-(1H-pyrazol-1-yl)pyridine; 1-methylcyclopropyl 4-[(1-{6-methanesulfonyl-5H,6H,7H,8H-pyrido[4,3-d]pyrimidin-2-yl}-1H-imidazol-4-yl)methyl]piperidine-1-carboxylate; 5-ethyl-2-{3-[(1-{6-methanesulfonyl-5H,6H,7H,8H-pyrido[4,3-d]pyrimidin-2-yl}piperidin-4-yl)methoxy]azetidin-1-yl}pyrimidine; 5-(4-{[(1-{6-methanesulfonyl-5H,6H,7H,8H-pyrido[4,3-d]pyrimidin-2-yl}azetidin-3-yl)oxy]methyl}piperidin-1-yl)-3-(propan-2-yl)-1,2,4-oxadiazole; 3-(4-{[(1-{6-methanesulfonyl-5H,6H,7H,8H-pyrido[4,3-d]pyrimidin-2-yl}azetidin-3-yl)oxy]methyl}piperidin-1-yl)-5-(propan-2-yl)-1,2,4-oxadiazole; 1-methylcyclopropyl(3R,4S)-4-{[(1-{6-methanesulfonyl-5H,6H,7H,8H-pyrido[4,3-d]pyrimidin-2-yl}azetidin-3-yl)oxy]methyl}-3-methoxypiperidine-1-carboxylate; 1-methylcyclopropyl(3R,4R)-4-{[(1-{6-methanesulfonyl-5H,6H,7H,8H-pyrido[4,3-d]pyrimidin-2-yl}azetidin-3-yl)oxy]methyl}-3-methylpiperidine-1-carboxylate; benzyl (2R,4R)-4-{[(1-{6-methanesulfonyl-5H,6H,7H,8H-pyrido[4,3-d]pyrimidin-2-yl}azetidin-3-yl)oxy]methyl}-2-methylpiperidine-1-carboxylate; benzyl 4-{[(1-{6-methanesulfonyl-5H,6H,7H,8H-pyrido[4,3-d]pyrimidin-2-yl}azetidin-3-yl)oxy]methyl}piperidine-1-carboxylate; 2-(5-ethylpyrimidin-2-yl)-5-[(1-{6-methanesulfonyl-5H,6H,7H,8H-pyrido[4,3-d]pyrimidin-2-yl}azetidin-3-yl)oxy]-1,2,3,4-tetrahydroisoquinoline; 5-ethyl-2-(4-{1-[(1-{6-methanesulfonyl-5H,6H,7H,8H-pyrido[4,3-d]pyrimidin-2-yl}azetidin-3-yl)oxy]ethyl}piperidin-1-yl)pyrimidine; 3-(2-{3-[1-(5-ethylpyrimidin-2-yl)piperidin-4-yl]propoxy}-5H,6H,7H,8H-pyrido[4,3-d]pyrimidine-6-sulfonyl)propan-1-ol; tert-butyl 4-(2-{[(3S)-1-{6-methanesulfonyl-5H,6H,7H,8H-pyrido[4,3d]pyrimidin-2-yl}pyrrolidin-3-yl]oxy}ethyl)piperidine-1-carboxylate; benzyl 2-{3-[1-(5-ethylpyrimidin-2-yl)piperidin-4-yl]propoxy}-5H,6H,7H,8H-pyrido[4,3-d]pyrimidine-6-carboxylate; and 5-ethyl-2-{4-[3-({6-methanesulfonyl-5H,6H,7H,8H-pyrido[4,3-d]pyrimidin-2-yl}oxy)propyl]phenyl}pyrimidine.

7. A pharmaceutical composition comprising a therapeutically effective amount of a compound of claim 1 in combination with a pharmaceutically acceptable excipient.

8. A method for modulating GPR119 activity, comprising administering to a system or a subject in need thereof, a therapeutically effective amount of the compound of claim 1 or pharmaceutically acceptable salts or pharmaceutical compositions thereof, thereby modulating said GPR119 activity.

9. The method of claim 8, wherein the compound of claim 1 directly contacts GPR119.

10. The method of claim 11, wherein the contacting occurs in vitro or in vivo.

11. A method for treating a disease or condition wherein modulation of GPR119 activity can prevent, inhibit or ameliorate the pathology and/or symptomology of the disease or condition, comprising administering to a subject a therapeutically effective amount of the compound of claim 1 or pharmaceutically acceptable salts or pharmaceutical compositions thereof.

12. The method of claim 11, wherein said disease or condition is selected from obesity, type 1 diabetes, type 2 diabetes mellitus, hyperlipidemia, idiopathic type 1 diabetes, latent autoimmune diabetes in adults, early-onset type 2 diabetes, youth-onset atypical diabetes, maturity onset diabetes of the young, malnutrition-related diabetes and gestational diabetes.

13. The method of claim 11, wherein said disease or condition is selected from coronary heart disease, ischemic stroke, restenosis after angioplasty, peripheral vascular disease, intermittent claudication, myocardial infarction, dyslipidemia, post-prandial lipemia, conditions of impaired glucose tolerance, conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.
